(19) United States
(12) Patent Application Publication
(10) Pub. No.: US 2007/0281969 A1
(43) Pub. Date: Dec. 6, 2007

(54) Niacin Receptor Agonists, Compositions Containing Such Compounds and Methods of Treatment

(76) Inventors: Steven L. Colletti, Princeton Junction, NJ (US); James R. Tata, Westfield, NJ (US); Hong C. Shen, West Windsor, NJ (US); Fa-Xiang Ding, Staten Island, NY (US); Jessica L. Frie, Princeton, NJ (US); Jason E. Imbriglio, Piscataway, NJ (US); Weichun Chen, Livingston, NJ (US)

Correspondence Address: MERCK AND CO., INC, P O BOX 2000, RAHWAY, NJ, 07065-0907, US

(21) Appl. No.: 11/791,183

(22) PCT Filed: Nov. 18, 2005
(86) PCT No.: PCT/US05/41962

§ 371 Date: May 17, 2007

Related U.S. Application Data
(60) Provisional application No. 60/630,281, filed on Nov. 23, 2004.

(51) Int. Cl. A61K 31/192; A61K 31/47; A61P 3/00; A61P 9/10; C07C 229/40; C07D 215/12

(52) U.S. Cl. 514/311; 514/568; 546/173; 562/456

(57) ABSTRACT

The present invention encompasses compounds of Formula (I); as well as pharmaceutically acceptable salts and hydrates thereof, that are useful for treating dyslipidemias. Pharmaceutical compositions and methods of use are also included.

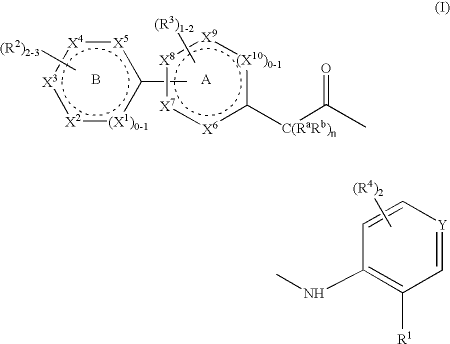

### BACKGROUND OF THE INVENTION

The present invention relates to biaryl compounds, compositions and methods of treatment or prevention in a mammal relating to dyslipidemias. Dyslipidemia is a condition wherein serum lipids are abnormal. Elevated cholesterol and low levels of high density lipoprotein (HDL) are associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules, such as steroids and bile acids, cholesterol in excess is known to contribute to cardiovascular disease. For example, cholesterol is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

The present invention relates to a compound represented by formula I: 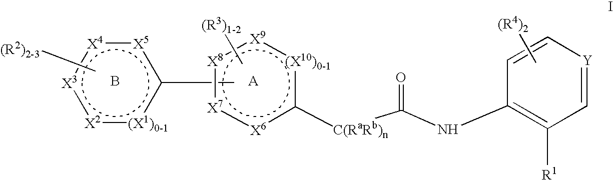
or a pharmaceutically acceptable salt or solvate thereof, wherein:

Y represents C or N;

R^{a} and R^{b} are independently H, C₁₋₃alkyl, haloC₁₋₃alkyl, OC₁₋₃alkyl, haloC₁₋₃alkoxy, OH or F;

n represents an integer of from 1 to 5;

R¹ represents -CO₂H, 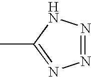 or —C(O)NHSO₂R^{c};

R^{c} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

X¹ through X¹⁰ represent C or a heteroatom selected from O, S and N, with up to 6 such heteroatoms present;

when X¹ is present, 0-2 of X¹- X⁵ represent N and 0-1 represent O or S;

when X¹ is absent, 0-3 of X²- X represent N and 0-1 represent O or S;

when X¹⁰ is present, 0-2 of X⁶- X¹⁰ represent N and 0-1 represent O or S;

when X¹⁰ is absent, 0-3 of X⁶-X⁹ represent N and 0-1 represent O or S;

when any of X¹- X¹⁰ is substituted, said X variable represents C;

when X¹⁰ is absent and at least one of X⁶-X⁹ is 0 and 2 of X⁶-X⁹ are N, and all of X¹ through X⁵ represent C, X³ is unsubstituted or is substituted with a member selected from the group consisting of: F, Br, I or a moiety selected from the group consisting of:

a) OH; CO₂H; CN; NH₂; S(O)₀₋₂R^{c};

wherein R^{c} is as previously defined;

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C 4 haloalkyl, OCO₂C₁₋₄alkyl, NH₂, NHC₁₋₄alklyl, N(C₁₋₄alkyl)₂, Hetcy, CN;

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;

and R′″ representing H or R″;

each R² represents H, F, Cl, Br, I or a moiety selected from the group consisting of (a), (b), (c), (d) or (e) above, or 1-2 R² groups are H, halo, C₁₋₆alkyl, OC₁₋₆alkyl, haloC₁₋₆alkyl or haloC₁₋₆alkoxy and the remaining R² groups are selected from the group consisting of (a), (b), (c), (d) or (e) above, or 1 R² group is a moiety selected from the group consisting of (a), (b), (c), (d) or (e) above, and the remaining R² groups are H or halo,

or

two R² groups can be taken in combination and represent a fused phenyl ring or ring B may represent a 5-6 membered fused heterocycle containing 0-1 of S, 0-2 of O, and containing 0-4 of N, and the remaining R² group is H, halo or a moiety selected from the group consisting of (a), (b), (c), (d) or (e) above,

said phenyl ring or fused heterocycle being fused at any available point and being optionally substituted with 1-3 halo, C₁₋₃alkyl or haloC₁₋₃alkyl groups, or 1-2 OC₁₋₃alkyl or haloC₁₋₃alkyl groups, or 1 moiety selected from the group consisting of:

a) OH; CO₂H; CN; NH₂; S(O)₀-₂R⁰;

b) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN;

c) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;

d) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Aryl and HAR,
said Aryl and HAR being further optionally substituted with 1-3 halo, C₁₋₄alkyl, C₁₋₄alkoxy, haloC₁₋₄alkyl and haloC₁₋₄alkoxy groups;
   (b) Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

each R³ represents H, halo, C₁₋₃alkyl, OC₁₋₃alkyl, haloC₁₋₃alkyl, haloC₁₋₃alkoxy, or S(O)_{y}C₁₋₃alkyl, wherein y is 0, 1 or 2, and

each R⁴ represents H, halo, methyl, or methyl substituted with 1-3 halo groups.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means a mono- or bicyclic aromatic ring or ring system containing at least one heteroatom selected from O, S and N, with each ring containing 5 to 6 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, quinolyl, indolyl, isoquinolyl, quinoxalinyl, quinazolinyl, naphthyridinyl, pyridinyl and the like. Heteroaryl also includes aromatic carbocyclic or heterocyclic groups fused to heterocycles that are non-aromatic or partially aromatic such as indolinyl, dihydrobenzofuranyl, dihydrobenzothiophenyl, dihydrobenzoxazolyl, and aromatic heterocyclic groups fused to cycloalkyl rings. Heteroaryl also includes such groups in charged form, e.g., pyridinium.

“Heterocycle” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocycle” include, but are not limited to, azetidinyl, pyrrolidinyl, piperidinyl, piperazinyl, imidazolidinyl, 2,3-dihydrofuro(2,3-b)pyridyl, tetrahydrofuranyl, benzoxazinyl, 1,4-dioxanyl, tetrahydrohydroquinolinyl, tetrahydroisoquinolinyl, dihydroindolyl, morpholinyl, thiomorpholinyl, tetrahydrothienyl and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heterocyclyl moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to a compound represented by formula I: 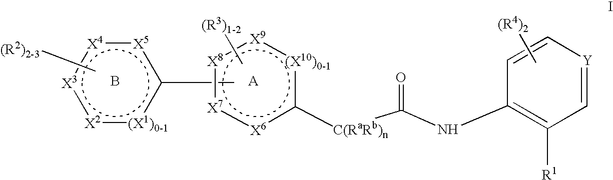
or a pharmaceutically acceptable salt or solvate thereof, wherein:

Y represents C or N;

R^{a} and R^{b} are independently H, C₁₋₃alkyl, haloC₁₋₃alkyl, OC₁₋₃alkyl, haloC₁₋₃alkoxy, OH or F;

n represents an integer of from 1 to 5;

R¹ represents —CO₂H, 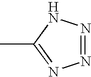
or —C(O)NHSO₂RC;

R^{c} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

X¹ through X¹⁰ represent C or a heteroatom selected from O, S and N, with up to 6 such heteroatoms present;

when X¹ is present, 0-2 of X¹- X⁵ represent N and 0-1 represent O or S;

when X¹ is absent, 0-3 of X²- X⁵ represent N and 0-1 represent O or S;

when X¹⁰ is present, 0-2 of X⁶-X¹⁰ represent N and 0-1 represent O or S;

when X¹⁰ is absent, 0-3 of X⁶-X⁹ represent N and 0-1 represent O or S;

when any of X¹-X¹⁰ is substituted, said X variable represents C;

when X¹⁰ is absent and at least one of X⁶-X⁹ is 0 and 2 of X⁶-X⁹ are N, and all of X¹ through X⁵ represent C, X³ is unsubstituted or is substituted with a member selected from the group consisting of: F, Br, I or a moiety selected from the group consisting of:

a) OH; CO₂H; CN; NH₂; S(O)₀₋₂R;

wherein R^{c} is as previously defined;

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, Hetcy, CN;

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NIHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

each R² represents H, F, Cl, Br, I or a moiety selected from the group consisting of (a), (b), (c), (d) or (e) above, or 1-2 R² groups are H, halo, C₁₋₆alkyl, OC₁₋₆alkyl, haloC₁₋₆alkyl or haloC₁₋₆alkoxy and the remaining R² groups are selected from the group consisting of (a), (b), (c), (d) or (e) above, or 1 R² group is a moiety selected from the group consisting of (a), (b), (c), (d) or (e) above, and the remaining R² groups are H or halo,

or

two R² groups can be taken in combination and represent a fused phenyl ring or ring B may represent a 5-6 membered fused heterocycle containing 0-1 of S, 0-2 of O, and containing 0-4 of N, and the remaining R² group is H, halo or a moiety selected from the group consisting of (a), (b), (c), (d) or (e) above,

said phenyl ring or fused heterocycle being fused at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of:

a) OH; CO₂H; CN; NH₂; S(O)₀₋₂R^{c};

b) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN;

c) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;

d) NR′C(O)R″, NR′SO₂R″, NR′CO₂R″ and NR′C(O)NR″R′″ wherein:
R¹ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Aryl and HAR,
said Aryl and HAR being further optionally substituted with 1-3 halo, C₁₋₄alkyl, C₁₋₄alkoxy, haloC₁₋₄alkyl and haloC₁₋₄alkoxy groups;
   (b) Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

each R³ represents H, halo, C₁₋₃alkyl, OC₁₋₃alk-yl, haloC₁₋₃alkyl, haloC₁₋₃alkoxy, or S(O)_{y}C₁₋₃alkyl, wherein y is 0, 1 or 2, and

each R⁴ represents H, halo, methyl, or methyl substituted with 1-3 halo groups.

A group of compounds that is of interest relates to compounds of formula I wherein Y represents C. Within this subset of compounds, all other variables are as originally defined with respect to formula I.

Another group of compounds that is of interest relates to compounds of formula I wherein R^{a} and R1 represent H or C₁₋₃alkyl. Within this subset of compounds, all other variables are as originally defined with respect to formula I.

In particular, another group of compounds that is of interest relates to compounds of formula I wherein one or both of R^{a} and R^{b} represent C₁₋₃alkyl. Within this subset of compounds, all other variables are as originally defined with respect to formula I.

More particularly, another group of compounds that is of interest relates to compounds of formula I wherein one or both of R^{a} and R^{b} represents methyl. Within this subset of compounds, all other variables are as originally defined with respect to formula I.

More particularly, another group of compounds that is of interest relates to compounds of formula I wherein R^{a} represents methyl. Within this subset of compounds, all other variables are as originally defined with respect to formula I.

Even more particularly, another group of compounds that is of interest relates to compounds of formula I wherein R^{a} and R^{b} both represent methyl. Within this subset of compounds, all other variables are as originally defined with respect to formula I.

Another group of compounds that is of interest relates to compounds of formula I wherein n represents an integer 1, 2 or 3. Within this subset of compounds, all other variables are as originally defined with respect to formula I.

More particularly, another group of compounds that is of interest relates to compounds of formula I wherein n represents 2. Within this subset of compounds, all other variables are as originally defined with respect to formula I.

Another group of compounds that is of interest relates to compounds of formula I wherein R¹ represents CO₂H or tetrazolyl. Within this subset of compounds, all other variables are as originally defined with respect to formula I.

More particularly, another group of compounds that is of interest relates to compounds of formula I wherein R¹ represents CO₂H. Within this subset of compounds, all other variables are as originally defined with respect to formula I.

Another group of compounds that is of interest relates to compounds of formula I wherein R⁴ represents H or halo. Within this subset of compounds, all other variables are as originally defined with respect to formula I.

Another group of compounds that is of interest relates to compounds of formula I wherein R⁴ represents halo. Within this subset of compounds, all other variables are as originally defined with respect to formula I.

Even more particularly, another group of compounds that is of interest relates to compounds of formula I wherein R⁴ represents fluoro. Within this subset of compounds, all other variables are as originally defined with respect to formula I.

Still more particularly, another group of compounds that is of interest relates to compounds of formula I wherein R⁴ represents fluoro at position 4 relative to the amide nitrogen. Within this subset of compounds, all other variables are as originally defined with respect to formula I.

Another group of compounds that is of interest relates to compounds of formula I wherein R⁴ represents H. Within this subset of compounds, all other variables are as originally defined with respect to formula I.

Another group of compounds that is of interest relates to compounds of formula I wherein ring A is selected from the group consisting of: phenyl, thiazole, oxadiazole, pyrazole and thiophene. Within this subset of compounds, all other variables are as originally defined with respect to formula I.

Another group of compounds that is of interest relates to compounds of formula I wherein ring A is selected from the group consisting of: thiazole, oxadiazole and pyrazole. Within this subset of compounds, all other variables are as originally defined with respect to formula I.

Another group of compounds that is of interest relates to compounds of formula I wherein ring A represents a phenyl or thiazolyl ring. Within this subset of compounds, all other variables are as originally defined with respect to formula I.

More particularly, another group of compounds that is of interest relates to compounds of formula I wherein ring A represents a phenyl ring. Within this subset of compounds, all other variables are as originally defined with respect to formula I.

Another group of compounds that is of interest relates to compounds of formula I wherein ring B is selected from the group consisting of: phenyl, pyridyl, pyrimidinyl, oxadiazolyl, furanyl and pyrazolyl. Within this subset of compounds, all other variables are as originally defined with respect to formula I.

Another group of compounds that is of interest relates to compounds of formula I wherein ring B is selected from the group consisting of: phenyl, pyridyl, oxadiazolyl and pyrazolyl. Within this subset of compounds, all other variables are as originally defined with respect to formula I.

Another group of compounds that is of interest relates to compounds of formula I wherein ring B represents a phenyl, pyridyl, pyrimidinyl, oxazolyl or furanyl ring. Within this subset of compounds, all other variables are as originally defined with respect to formula I.

More particularly, another group of compounds that is of interest relates to compounds of formula I wherein ring B represents a phenyl or pyridyl ring. Within this subset of compounds, all other variables are as originally defined with respect to formula I.

Yet another group of compounds that is of particular interest relates to compounds of formula I wherein rign B represents pyridyl. Within this subset of compounds, all other variables are as originally defined with respect to formula I.

Another group of compounds that is of interest relates to compounds of formula I wherein each R² represents H, F, Cl, or a moiety selected from the group consisting of a) OH; CO₂H; CN; NH₂;

b) C₁₋₃ alkyl and OC₁₋₃alkyl, said group being optionally substituted with 1-3 groups, 1-3 of which are halo and 1 of which is selected from: OH, CO₂H, CO₂C₁₋₄alkyl, CO₂C₁₋₄ haloalkyl, NH₂, NHCH₃ and N(CH₃)₂;

c) NHCH₃ and N(CH₃)₂;

d) C(O)NH₂, C(O)NHCH₃, C(O)N(CH₃)₂, C(O)NHOCH₃ and C(O)N(CH₃)(OCH₃);

e) NR′C(O)R″, NR′SO₂R″, NR′CO₂R″ and NR′C(O)NR″R′″ wherein:
R′ represents H, CH₃ or haloC₁₋₂alkyl,
R″ represents (a) C₁₋₂alkyl optionally substituted with 1-3 groups, 0-3 of which are halo, and 0-1 of which are selected from the group consisting of: OCH₃, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl,
said Aryl being further optionally substituted with 1-3 halo, CH₃, OCH₃, haloC₁₋₂alkyl and haloC₁₋₂alkoxy groups;
   (b) Aryl optionally substituted with 1-3 halo, CH₃, OCH₃, C₁₋₂alkoxy, haloC₁₋₂alkyl and haloC₁₋₂alkoxy groups;
and R′″ represents H or R″.
   Within this subset of compounds, all other variables are as originally defined with respect to formula I.

Another group of compounds that is of interest relates to compounds of formula I wherein two R² taken in combination and represent a fused phenyl ring or a 5-6 membered fused heterocycle containing 0-1 of S, 0-2 of O, and containing 0-4 of N, and the remaining R² group is H, F, Cl, or a moiety selected from the group consisting of

a) OH; CO₂H; CN; NH₂;

b) C₁₋₃ alkyl and OC₁₋₃alkyl, said group being optionally substituted with 1-3 groups, 1-3 of which are halo and 1 of which is selected from: OH, CO₂H, CO₂C₁₋₄alkyl, CO₂C₁₋₄haloalkyl, NH₂, NHCH₃ and N(CH₃)₂;

c) NHCH₃ and N(CH₃)₂;

d) C(O)NH₂, C(O)NHCH₃, C(O)N(CH₃)₂, C(O)NHOCH₃ and C(O)N(CH₃)(OCH₃);

e) NR′C(O)R″, NR′SO₂R″, NR′CO₂R″ and NR′C(O)NR″R′″ wherein:
R′ represents H, CH₃ or haloC₁₋₂alkyl,
R″ represents (a) C₁₋₂alkyl optionally substituted with 1-3 groups, 0-3 of which are halo, and 0-1 of which are selected from the group consisting of: OCH₃, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl,
said Aryl being further optionally substituted with 1-3 halo, CH₃, OCH₃, haloC₁₋₂alkyl and haloC₁₋₂alkoxy groups;
   (b) Aryl optionally substituted with 1-3 halo, CH₃, OCH₃, C₁₋₂alkoxy, haloC₁₋₂alkyl and haloC₁₋₂alkoxy groups;
and R′″ represents H or R″;

said fused phenyl ring or heterocycle being fused at any available point and being optionally substituted with 1-3 halo, C₁₋₂alkyl or haloC₁₋₂alkyl groups, or 1-2OC₁₋₂alkyl or haloOC₁₋₂alkyl groups, or 1 moiety selected from the group consisting of:

a) OH; CO₂H; CN; NH₂;

b) NHCH₃ and N(CH₃)₂, the alkyl portions of which are optionally substituted with 1-3 groups, 1-3 of which are halo and 1 of which is selected from: OH, CO₂H, CO₂C₁₋₂alkyl, CO₂C₁₋₂ haloalkyl, OCO₂C₁₋₂alkyl, NH₂, NHCH₃, N(CH₃)₂, CN;

c) C(O)NH₂, C(O)NHCH₃, C(O)N(CH₃)₂, C(O)NHOCH₃ and C(O)N(CH₃)(OCH₃), the alkyl portions of which are optionally substituted as set forth in (b) above;

d) NR′C(O)R″, NR′SO₂R″, NR′CO₂R″ and NR′C(O)NR″R′″ wherein:
R′ represents H, C₁₋₂alkyl or haloC₁₋₂alkyl,
R″ represents (a) C₁₋₈alkyl optionally substituted with 1-4 groups, 0-4 of which are halo, and 0-1 of which are selected from the group consisting of: OC₁₋₃alkyl, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl HAR,
said Aryl being further optionally substituted with 1-3 halo, CH₃, OCH₃, haloC₁₋₂alkyl and haloC₁₋₂alkoxy groups;
   (b) Aryl or HAR, said Aryl and HAR being further optionally substituted with 1-3 halo, CH₃, OCH₃, haloC₁₋₂alkyl and haloC₁₋₂alkoxy groups;
and R′″ representing H or R″.

More particularly, another group of compounds that is of interest relates to compounds of formula I wherein one R² represents H, OH, CF₃, NH₂, Cl, Me, OMe, F, MeSO₂— or HOCH₂—. Within this subset of compounds, all other variables are as originally defined with respect to formula I.

Even more particularly, another group of compounds that is of interest relates to compounds of formula I wherein one R² represents H, OH, CF₃, Cl, Me, OMe, F, MeSO₂— or HOCH₂—. Within this subset of compounds, all other variables are as originally defined with respect to formula I.

Even more particularly, another group of compounds that is of interest relates to compounds of formula I wherein one R² represents OH or NH₂. Within this subset of compounds, all other variables are as originally defined with respect to formula I.

Examples of compounds falling within the present invention are set forth below in Table 1.

TABLE 1

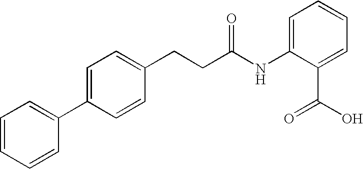

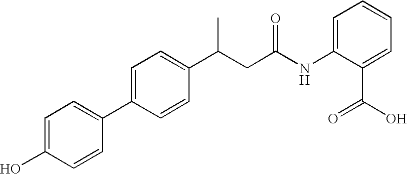

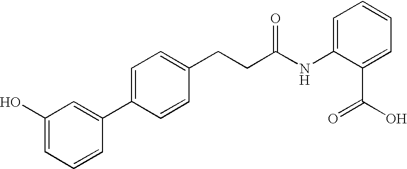

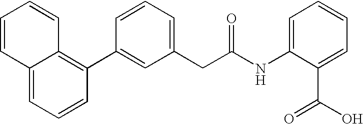

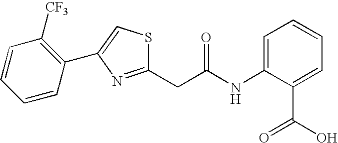

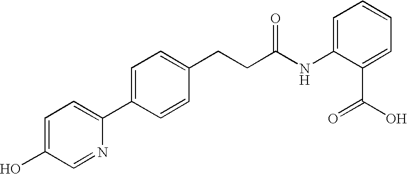

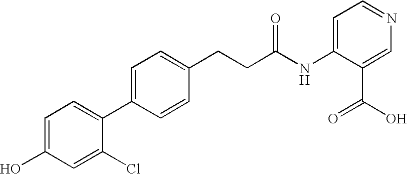

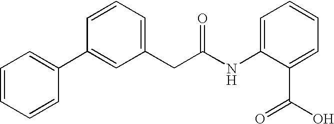

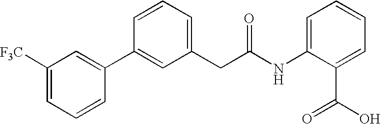

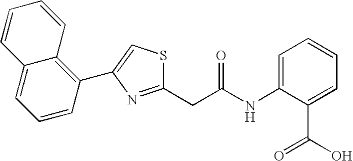

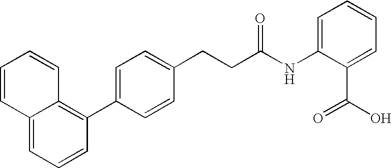

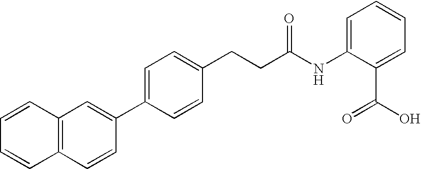

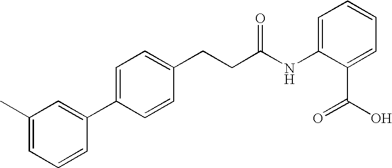

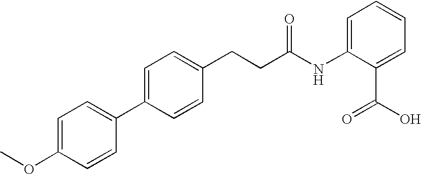

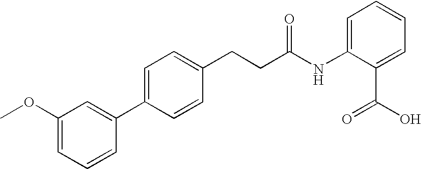

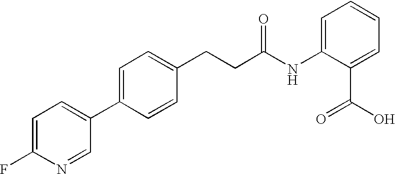

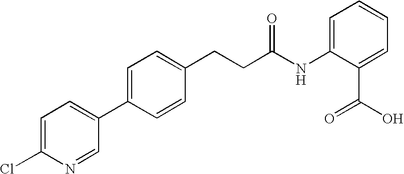

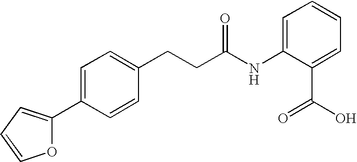

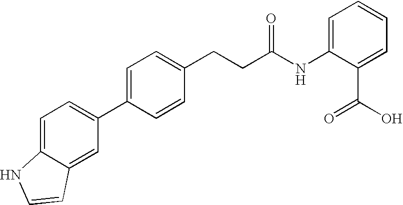

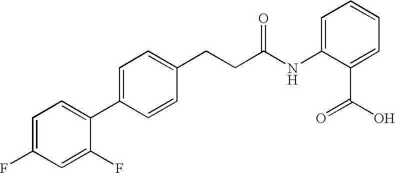

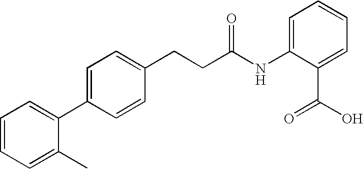

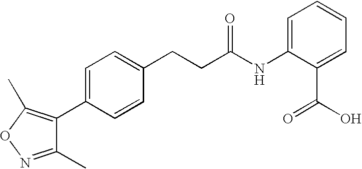

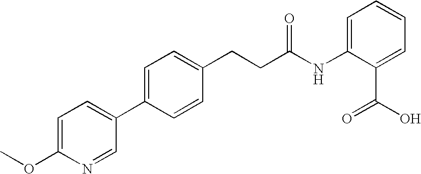

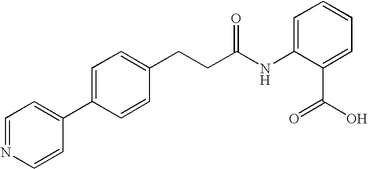

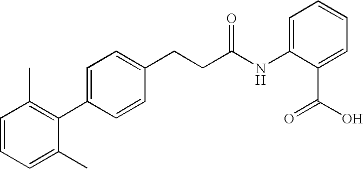

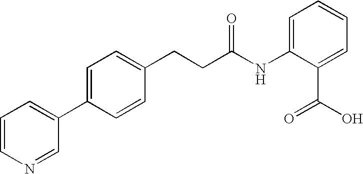

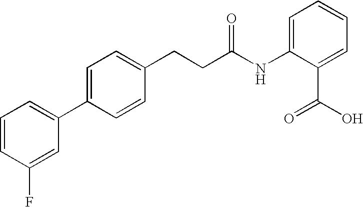

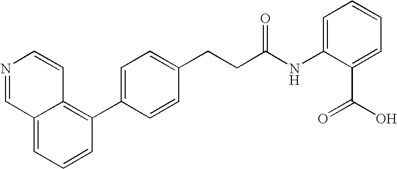

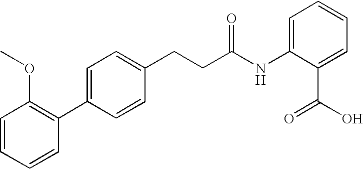

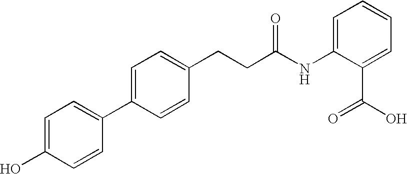

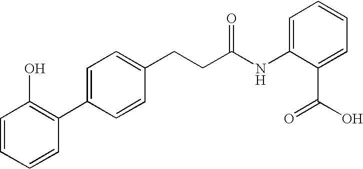

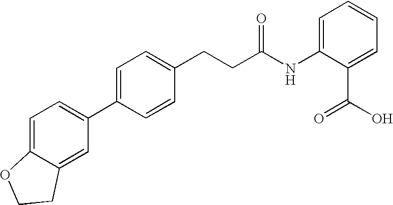

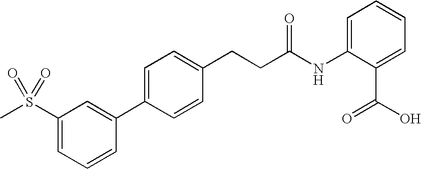

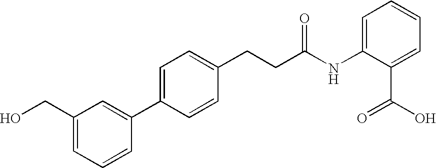

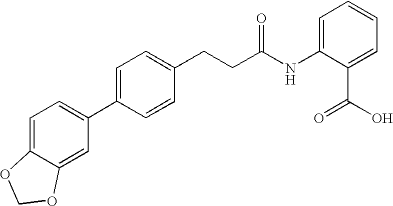

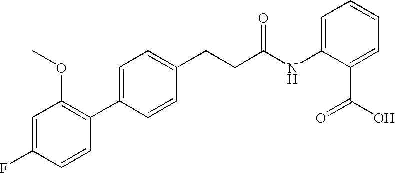

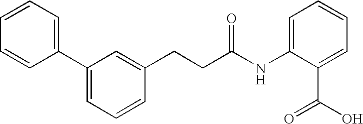

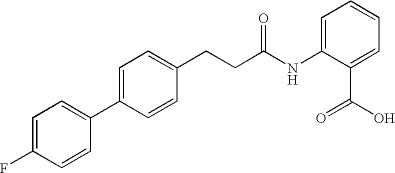

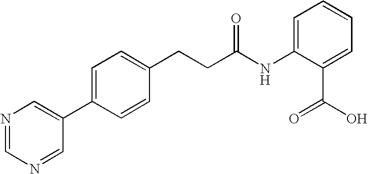

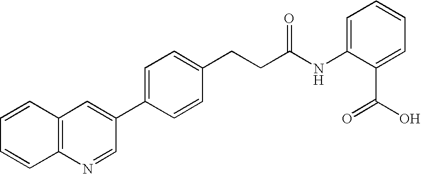

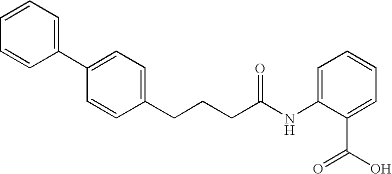

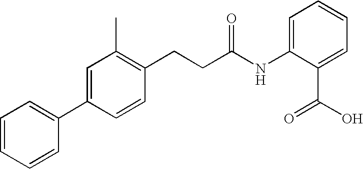

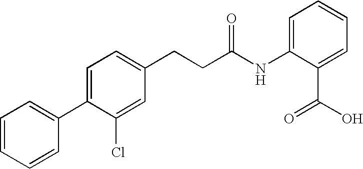

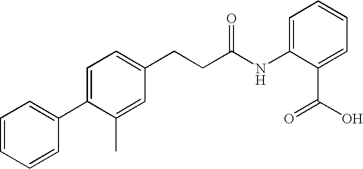

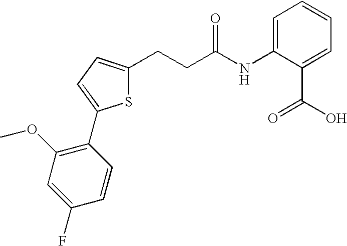

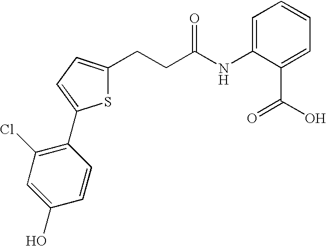

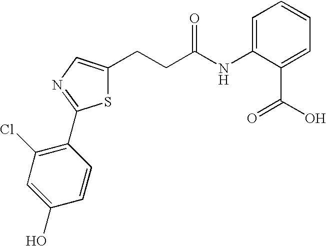

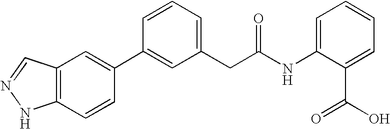

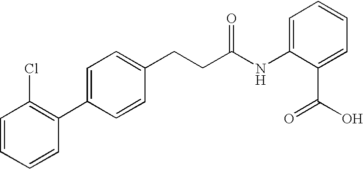

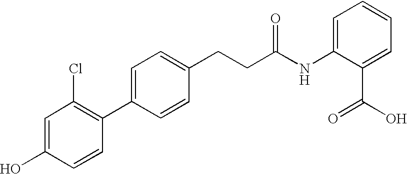

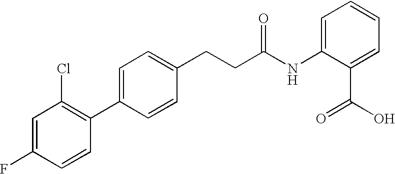

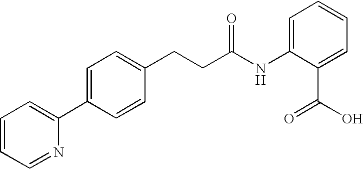

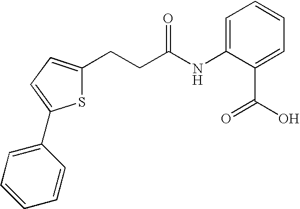

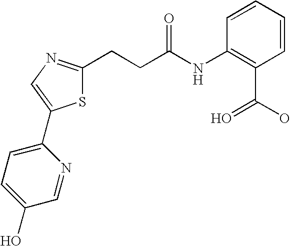

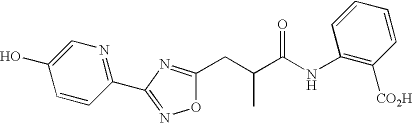

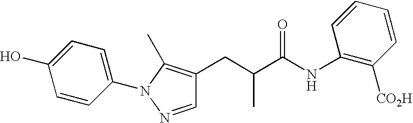

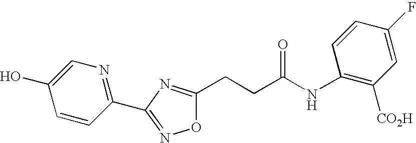

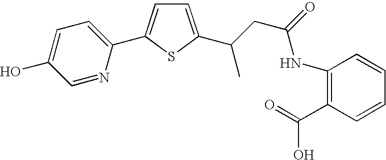

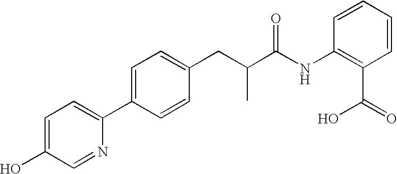

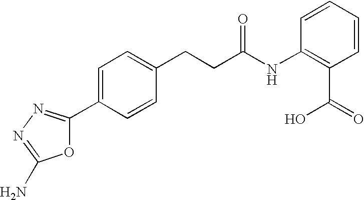

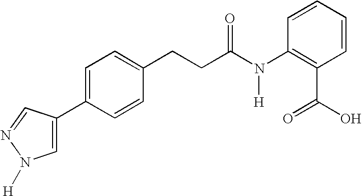

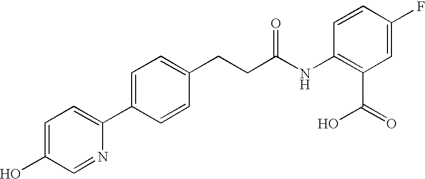

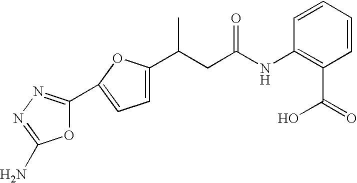

Pharmaceutically acceptable salts and solvates thereof are included as well.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

Dosing Information

The dosages of compounds of formula I or a pharmaceutically acceptable salt or solvate thereof vary within wide limits. The specific dosageregimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, famesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3 (R)-[3 (S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taling insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

Compound A

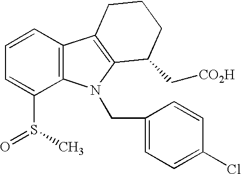

Compound B

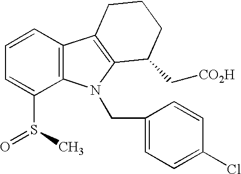

Compound C

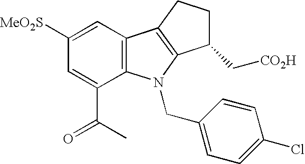

Compound D

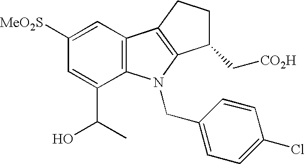

Compound E

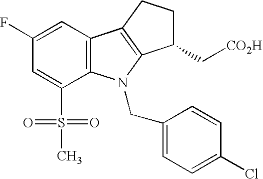

Compound F

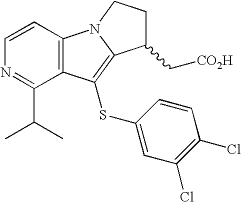

Compound G

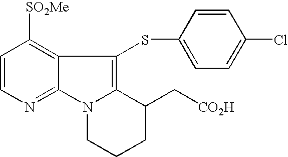

Compound H

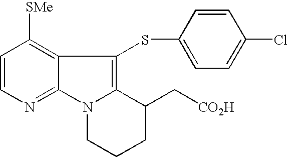

Compound I

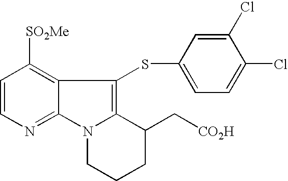

Compound J

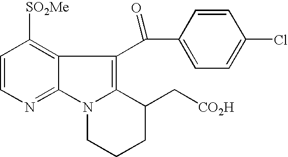

Compound K

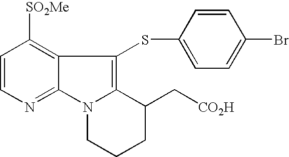

Compound L

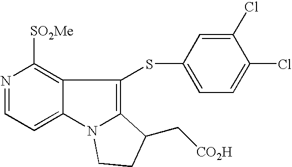

Compound M

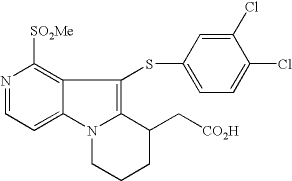

Compound N

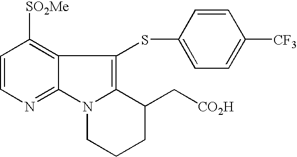

Compound O

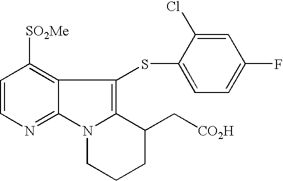

Compound P

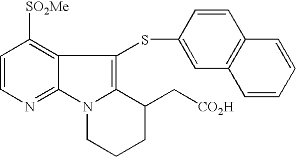

Compound Q

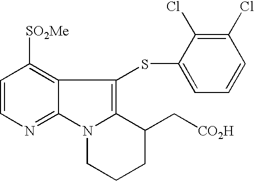

Compound R

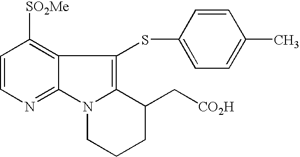

Compound S

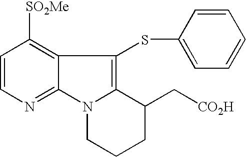

Compound T

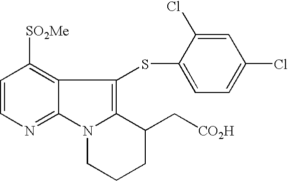

Compound U

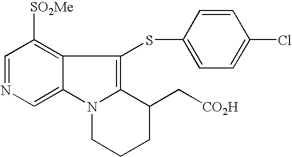

Compound V

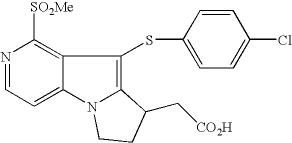

Compound W

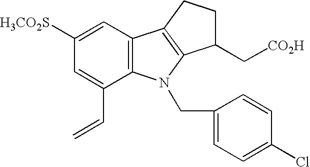

Compound X

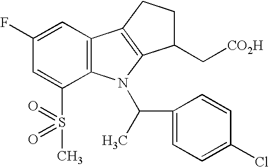

Compound Y

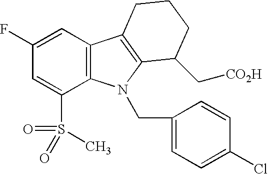

Compound Z

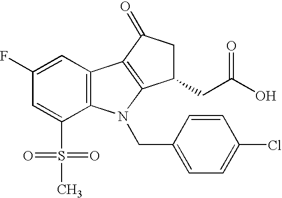

Compound AA

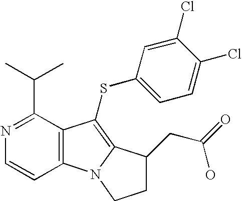

Compound AB

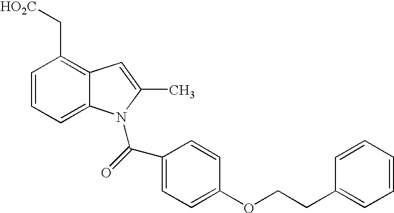

Compound AC

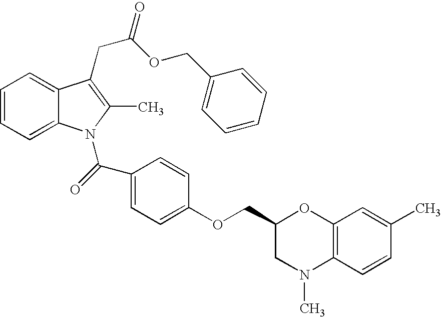

Compound AD

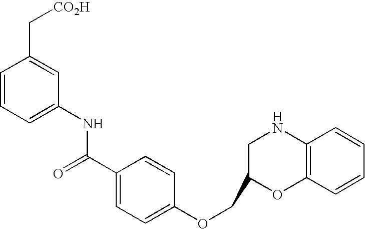

Compound AE

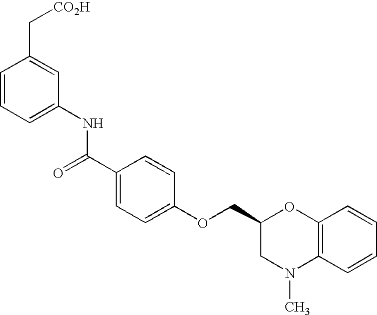

Compound AF

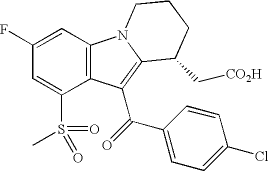

Compound AG

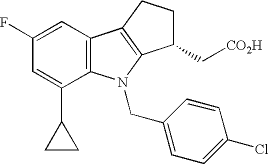

Compound AH

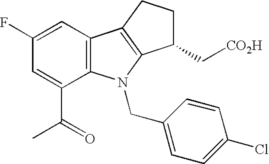

Compound AI

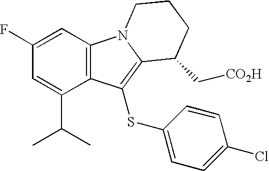

Compound AJ

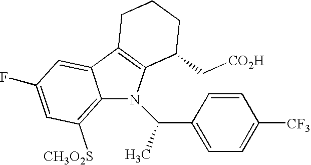

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The compounds of the invention also include esters that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents,e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventin or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

Methods of Synthesis for Compounds of Formula I

Compounds of formula I have been prepared by the following reaction schemes. It is understood that other synthetic approaches to these structure classes are conceivable to one skilled in the art. Therefore these reaction schemes should not be construed as limiting the scope of the invention. All substituents are as defined above unless indicated otherwise.

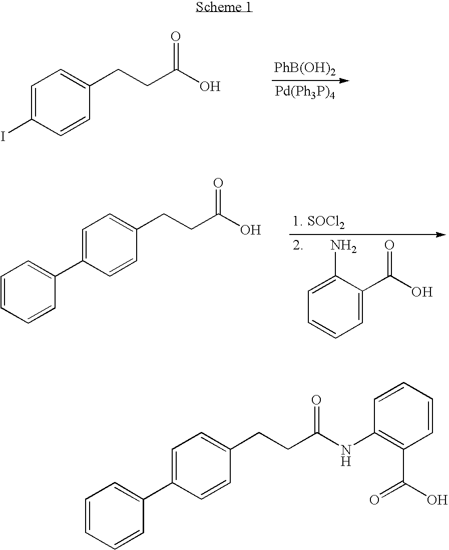

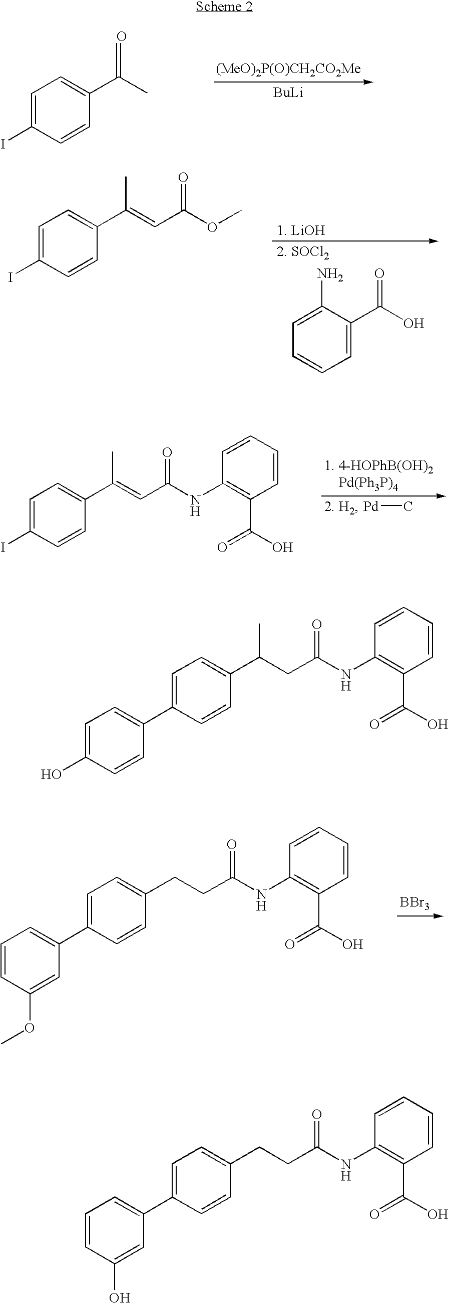

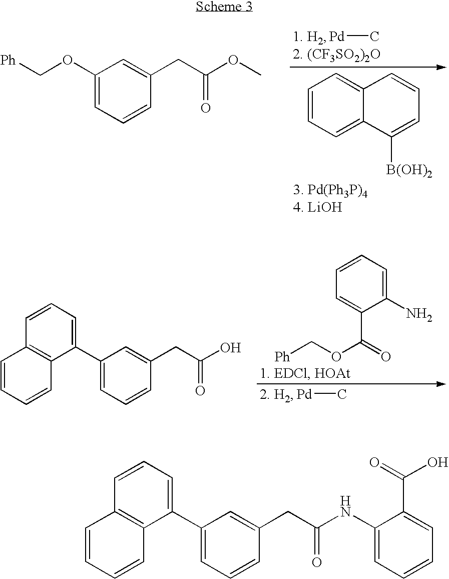

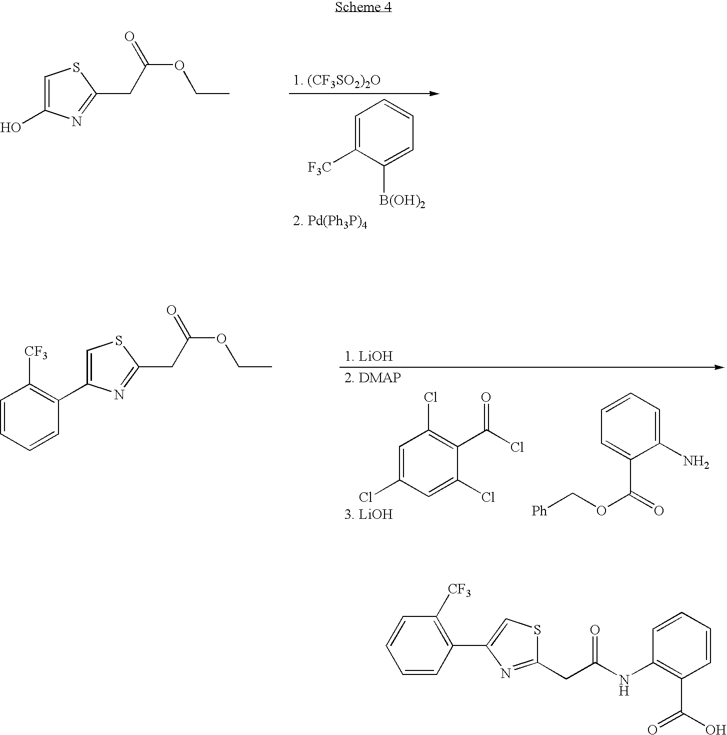

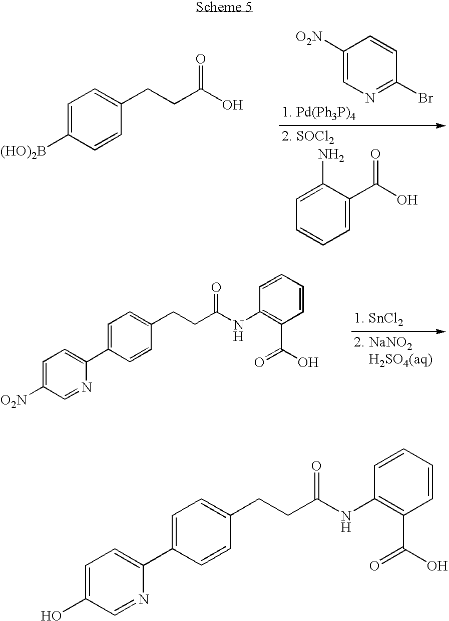

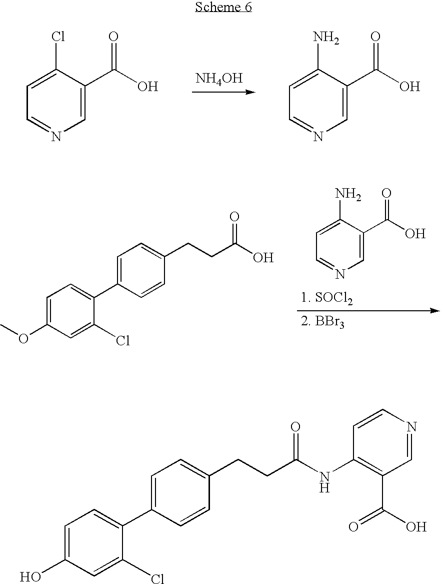

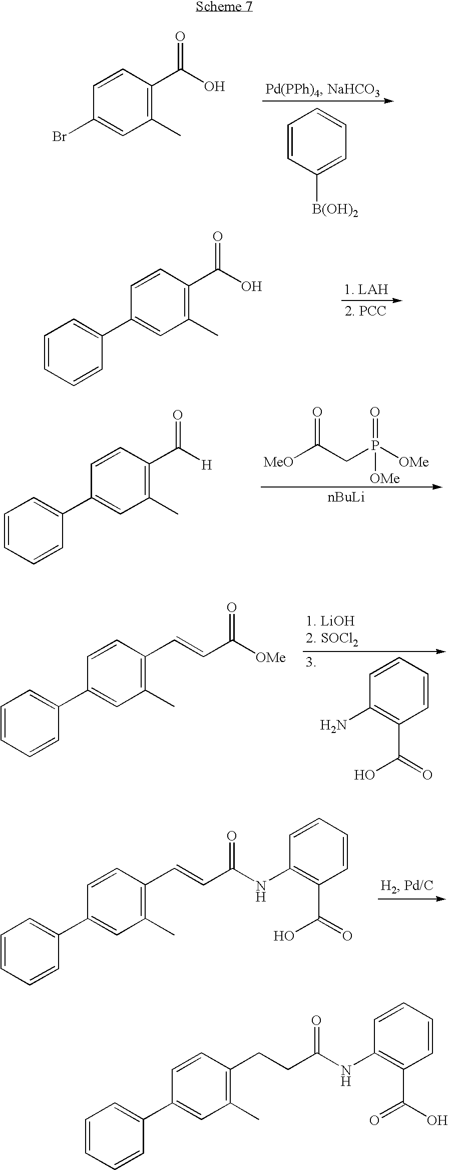

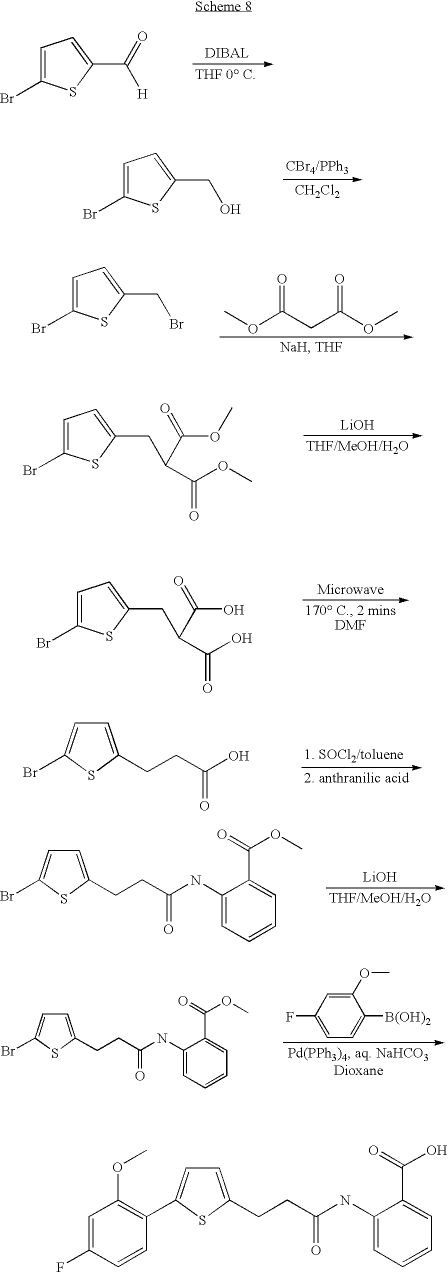

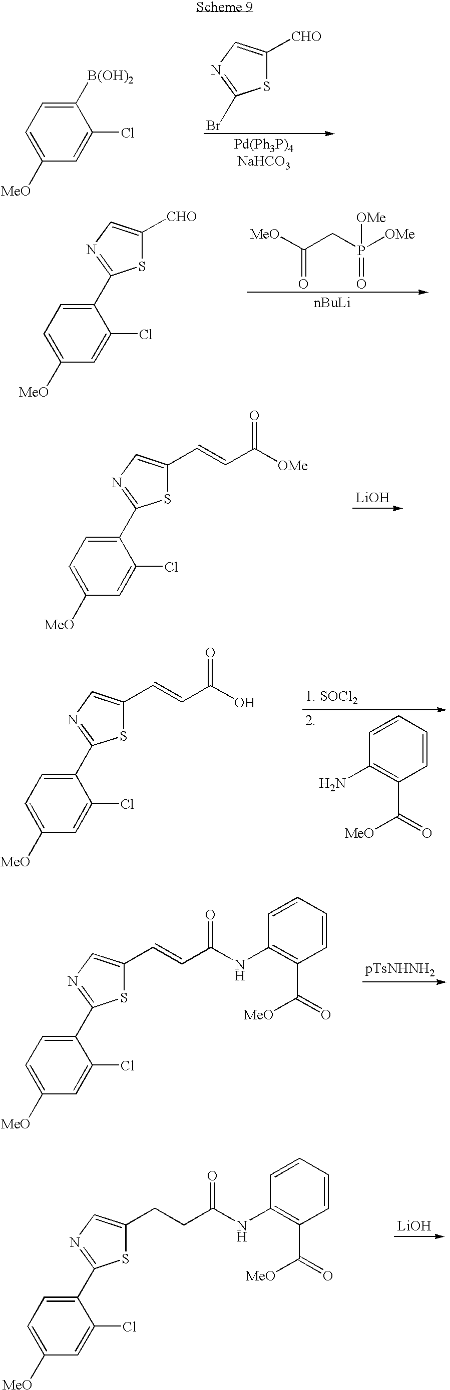

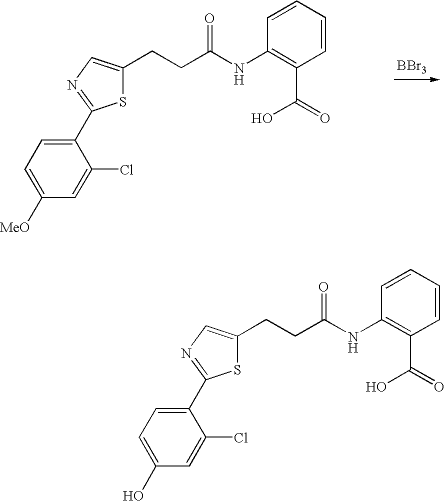

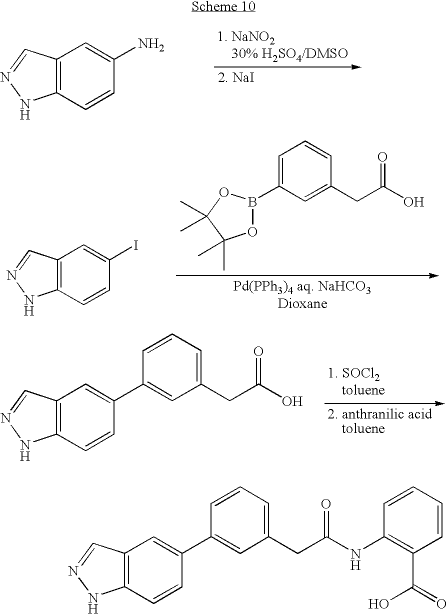

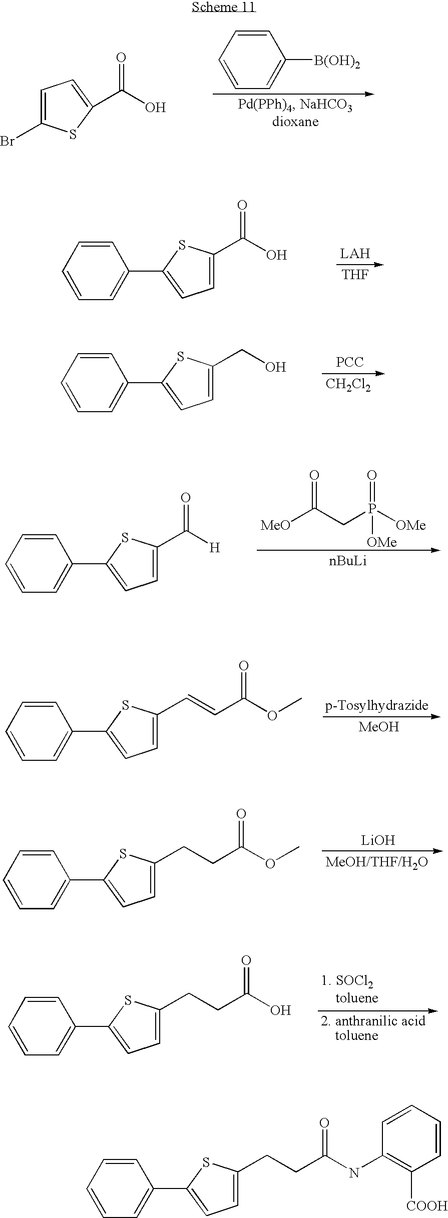

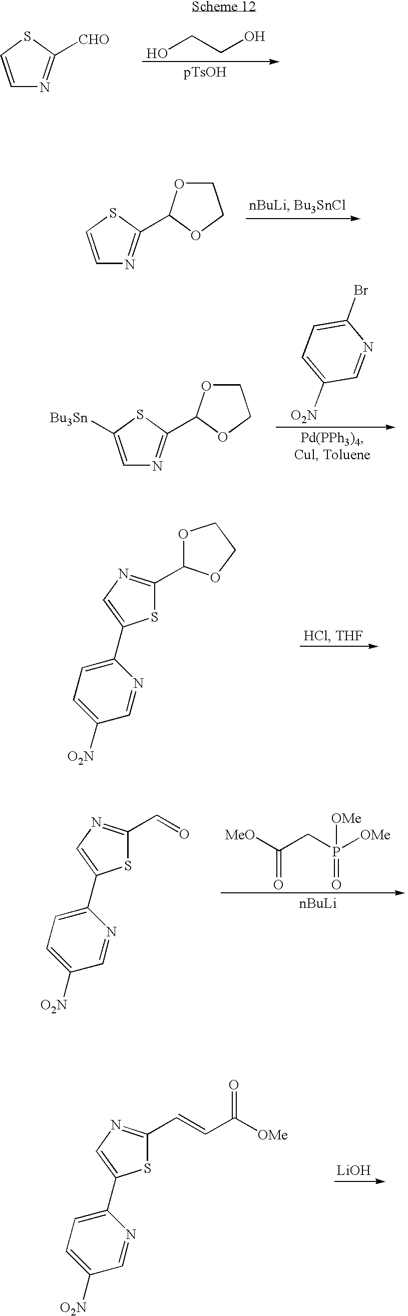

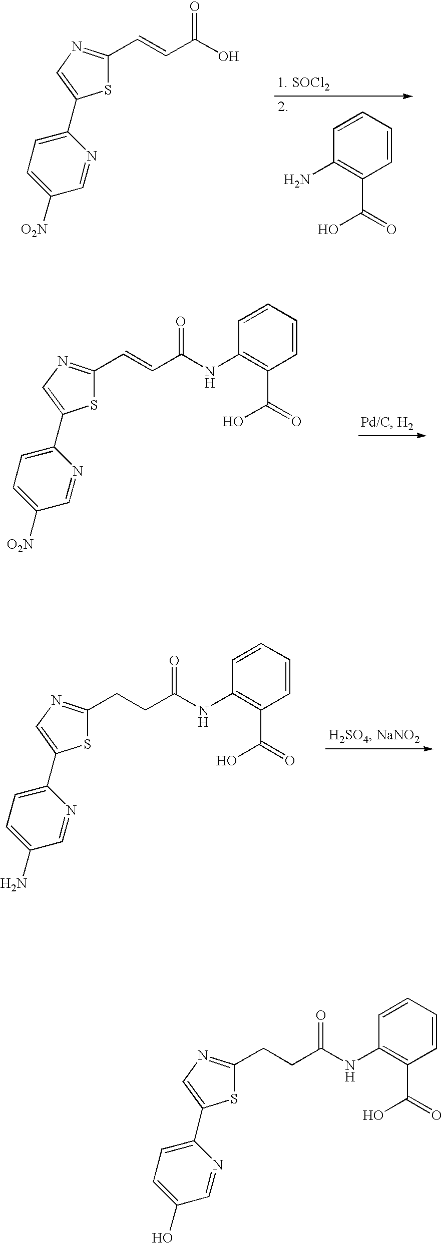

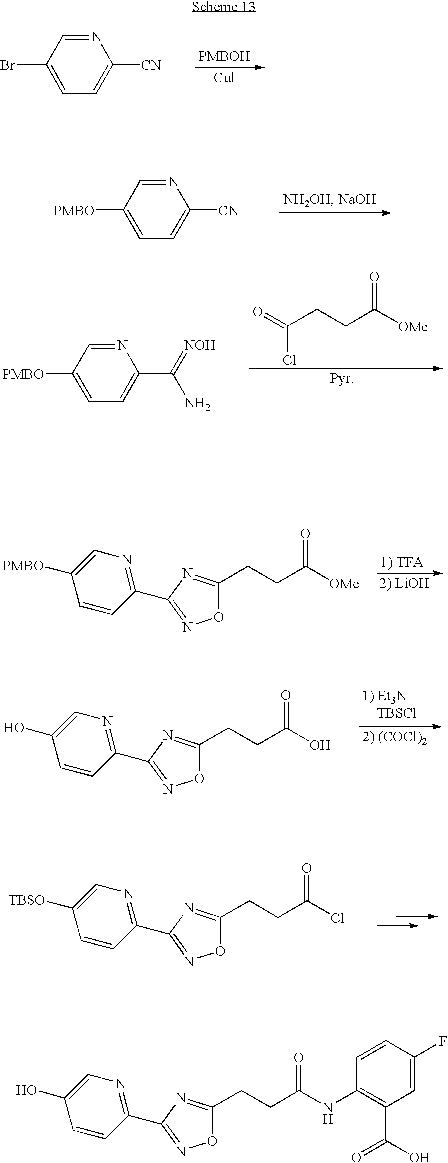

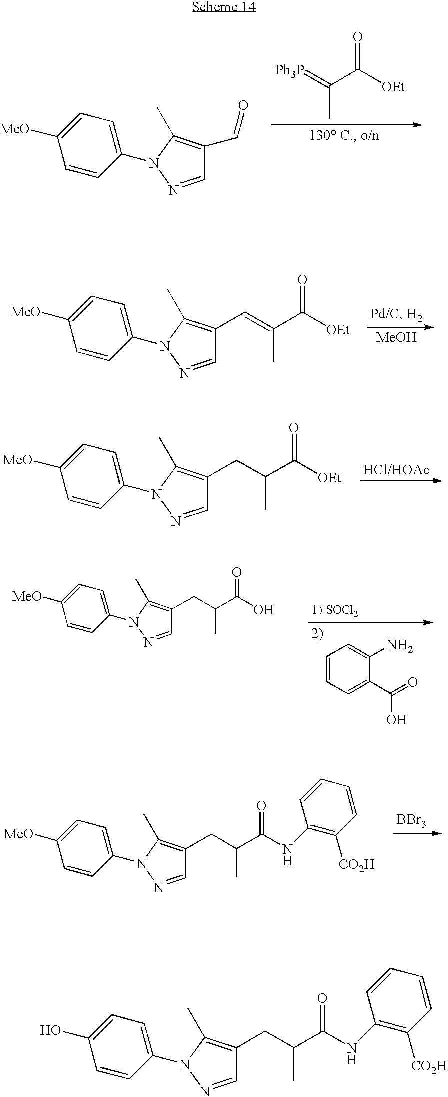

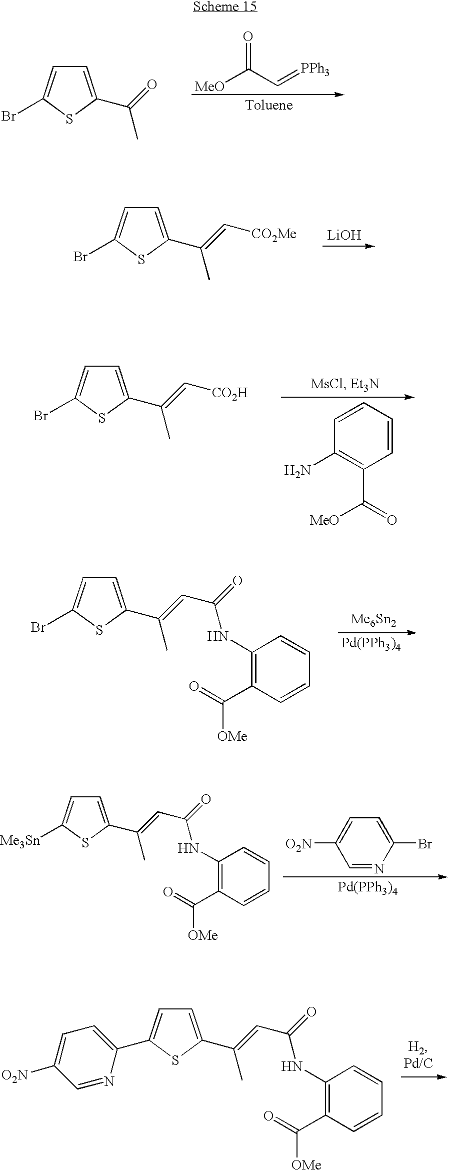

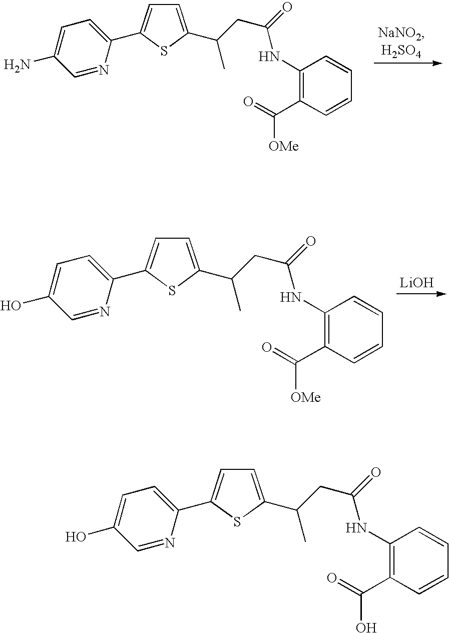

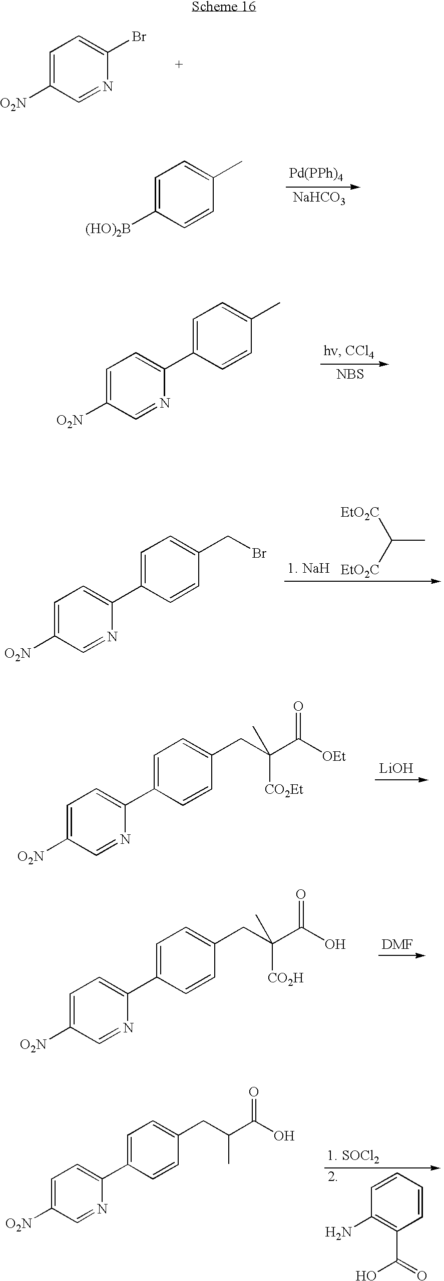

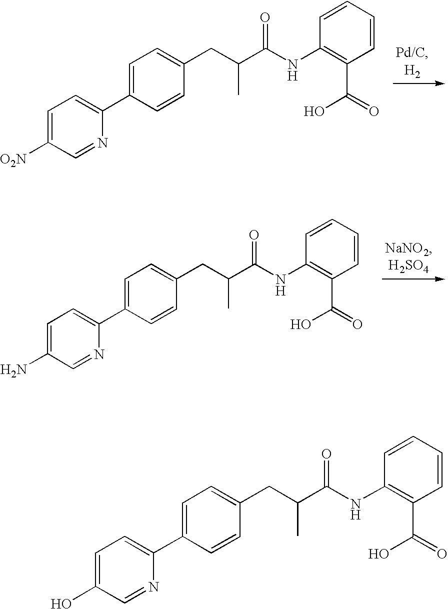

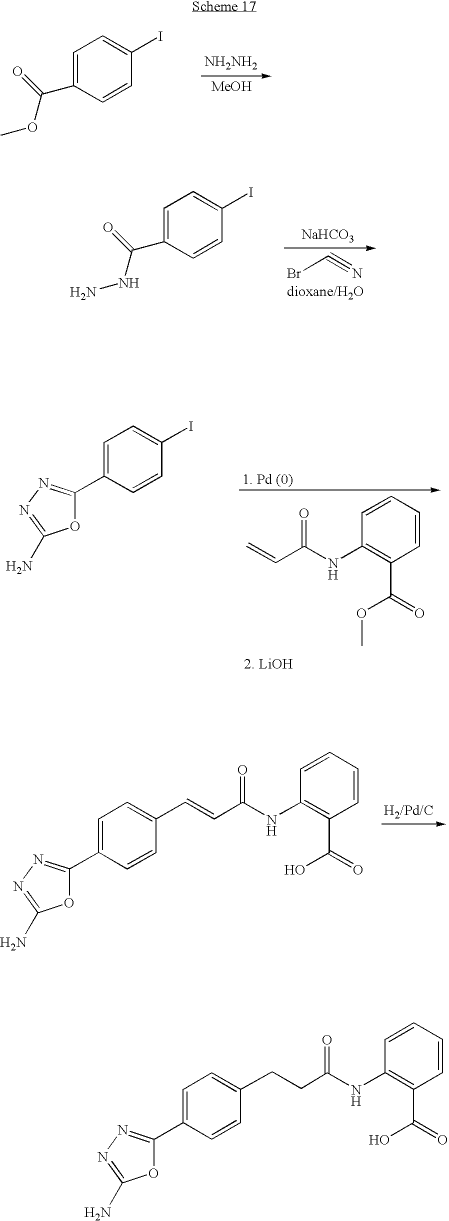

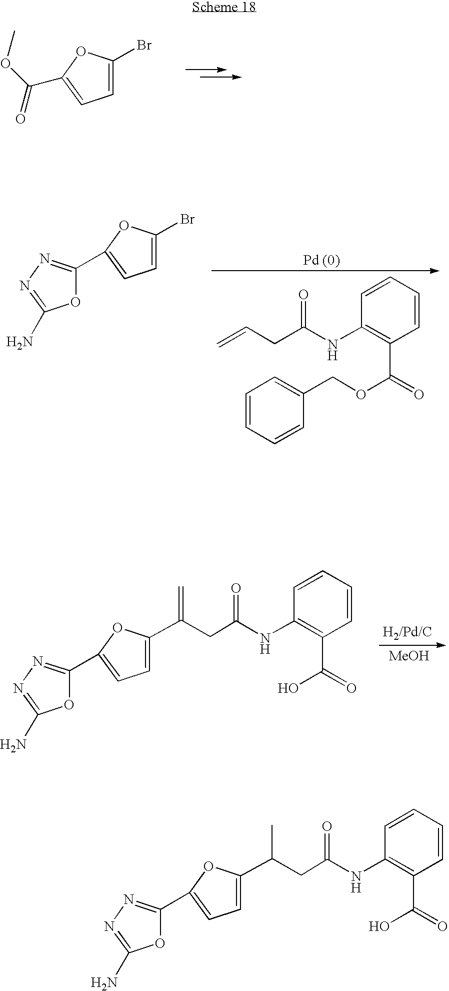

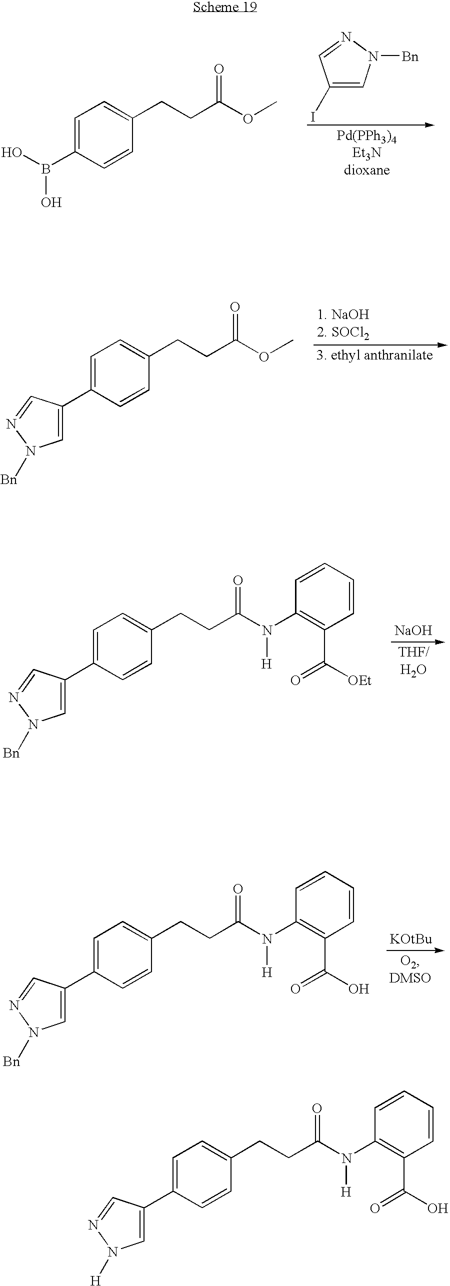

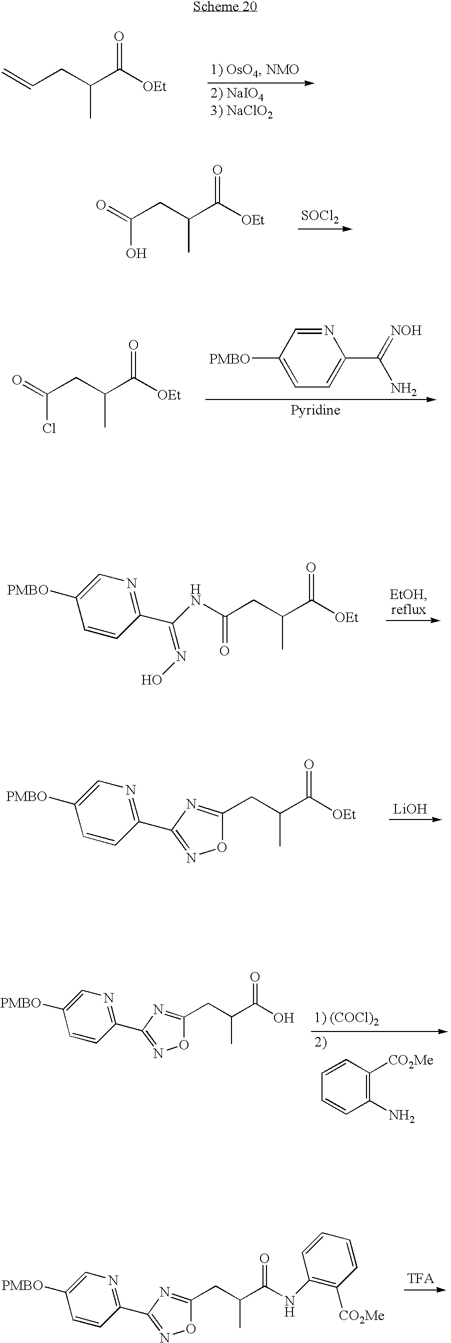

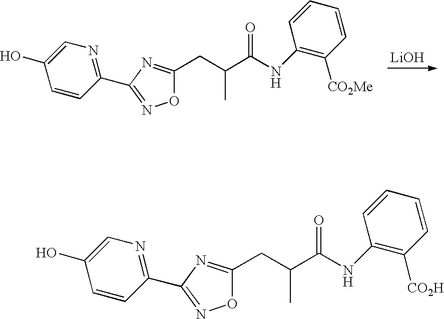

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) the structure of all final compounds was assured by at least one of the following techniques: MS or proton nuclear magnetic resonance (1H NMR) spectrometry, and the purity was assured by at least one of the following techniques: TLC or HPLC;

(v) 1H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vi) MS data were recorded on a Waters Micromass unit, interfaced with a Hewlett-Packard (Agilent 1100) HPLC instrument, and operating on MassLynx/OpemLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES- was 1-2 mL/min, 10-95% B linear gradient over 5.5 min (B=0.1% formic acid—acetonitrile, A=0.1% formic acid−water), Waters XTerra C18−3.5 um−50×3.0 mmID and diode array detection;

(vii) the purification of compounds by preparative reverse phase HPLC (RPHPLC) was conducted on either a Waters Symmetry Prep C18−5 um−30×100 mmID, or a Waters Atlantis Prep dC18−5 um−20×100 mmID; 20 mL/min, 10-100% B linear gradient over 15 min (B=005% TFA-acetonitrile, A=0.05% TFA-water), and diode array detection on a Varian system;

(viii) the automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(ix) the purification of compounds by preparative thin layer chromatography (PTLC) was conducted on 20×20 cm glass prep plates coated with silica gel, commercially available from Analtech, or column chromatography was carried out on a glass silica gel column using Kieselgel 60, 0.063-0.200 mm (Merck), or a Biotage cartridge system;

(x) chemical symbols have their usual meanings; the following abbreviations have also been used v (volume), w (weight), b.p. (boiling point), m.p. (melting point), L (litre(s)), ML (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which produces 50% of the maximum possible efficacy or response), uM (micromolar), mM (nanomolar).

(xi) the definitions of acronyms are as follows:
rt or RT is room temperature;
THF is tetrahydrofuran;
DMSO is dimethylsulfoxide;
DMF is dimethylformamide;
DIBAL is diisobutylaluminum hydride;
DCM is dichloromethane (methylene chloride);
DME is dimethoxyethane.

### Example 1

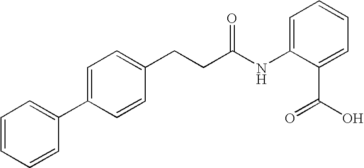

Commercially available 3-(4-iodophenyl)propionic acid (200 mg, 0.72 mmol) was combined with phenyl boronic acid (177 mg, 1.45 mmol), catalytic tetrakis-(triphenylphosphine)palladium (20 mg), and saturated aqueous sodium bicarbonate (1M, 1.45 mL, 1.45 mmol) in (1:1) dioxane-ethanol (5 mL). The reaction mixture was heated at 100° C. overnight, cooled to room temperature, filtered, and concentrated in vacuo. The residue was purified via preparative RPHPLC to give the biaryl propionic acid intermediate. This acid (59 mg, 0.26 mmol) was diluted into toluene (5 mL), treated with thionyl chloride (0.5 mL), and the reaction mixture refluxed overnight. The solvent was evaporated, and the acid chloride product was azeotroped with toluene twice. A third of the remaining yellow oil was diluted into toluene (2 mL), then treated with anthranilic acid (71 mg, 0.52 mmol), and the reaction mixture was heated at reflux for 2 h. The mixture was then cooled to room temperature, concentrated in vacuo, and purified via preparative RPHPLC to give the desired product: ¹H NMR (acetone-d₆, 500 MHz) δ 8.76 (d, 1H), 8.10 (d, 1H), 7.63 (m, 5H), 7.46 (m, 5H), 7.33 (t, 1H), 7.15 (t, 1H), 3.10 (t, 2H), 2.81 (t, 2H); LCMS m/z 344 (M⁺−1).

### Example 2

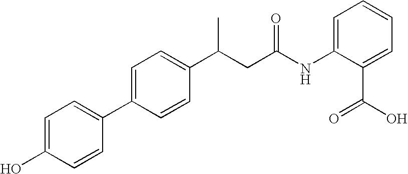

Trimethyl phosphonoacetate (890 mg, 4.88 mmol) was diluted into tetrahydrofuran (10 mL), cooled to 0° C., and deprotonated with n-butyl]ithium (1.6M, 3.7 mL, 5.86 mmol). The reaction mixture was aged 30 min, and then treated with a tetrahydrofuran (5 mL) solution of commercially available 4-iodoacetophenone (1 g, 4.07 mmol). The reaction mixture was then warmed to room temperature, maintained for 1 h, warmed further to 50° C. for 3 h, quenched with water, and partitioned with ethyl acetate. The organic phase was separated, dried over sodium sulfate, and concentrated in vacuo. The product was purified by flash column chromatography (Biotage, SiO₂, 5% EtOAc-hexane) to provide the methyl enoate intermediate. This methyl ester (690 mg, 2.28 minol) was saponified with LiOH (1N, 10 mL) in (3:1:1) THF-MeOH—H₂O (20 mL) overnight. The reaction mixture was then concentrated in vacuo, diluted with water (20 mL), extracted with chloroform (15 mL), the aqueous phase separated, acidified with conc. HCl to pH 3, and then extracted with 30% isopropanol-chloroform (50 mL). The organic partition was separated, dried over anhydrous sodium sulfate, concentrated in vacuo, and the crude solid was used for the next step without purification. This intermediate enoic acid (590 mg, 2.05 mmol) was activated with thionyl chloride and coupled with anthranilic acid in a similar manner as described in EXAMPLE 1 to provide the desired iodoacrylamide intermediate. This iodide (30 mg, 0.074 mmol) was coupled with 4-hydroxyphenyl boronic acid under conditions described in EXAMPLE 1 to provide the biaryl product. This biaryl acrylamide intermediate (5 mg, 0.013 mmol) was treated with catalytic palladium on carbon in methanol (2 mL), and hydrogenated at 1 atmosphere with a hydrogen-filled balloon for 2 h. The reaction mixture was filtered over celite, concentrated in vacuo, and purified via preparative RPHPLC to give the desired product: ¹H NMR (acetone-d₆, 500 MHz) δ 11.3 (s, 1H), 8.72 (d, 1H), 8.09 (dd, 1H), 7.51 (m, 5H), 7.40 (d, 2H), 7.12 (t, 1H), 6.91 (m, 2H), 3.42(m, 1H), 2.75 (m, 2H), 1.37(d, 3H); LCMS m/z 374 (M⁺−1).

### Example 3

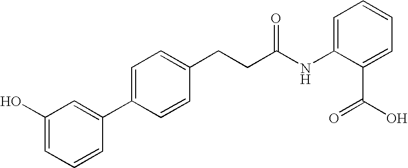

EXAMPLE 3 can be prepared from its methyl ether derivative EXAMPLE 15 (5 mg, 0.013 mmol), by demethylation with boron tribromide (0.3 mL) in methylene chloride (2 mL). The reaction mixture was aged 2 h, quenched with water, reduced in volume by evaporation in vacuo, and purified directly by preparative RPHPLC to give the desired product: 1H NMR (acetone-d6, 500 MHz) o 11.3 (s, 1H), 8.76 (d, 1H), 8.11 (d, 1H), 7.59 (m, 1H), 7.54 (d, 2H), 7.39 (d, 2H), 7.26 (t, 1H), 7.15 (t, 1H), 7.10 (t, 1H), 6.82 (d, 1H), 3.09 (t, 2H), 2.81 (t, 2H); LCMS m/z 360 (M⁺−1).

### Example 4

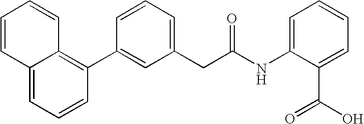

Commercially available 3-benzyloxyphenylacetic acid (1 g, 3.9 mmol) was treated with catalytic palladium on carbon (Degussa) in methanol, and hydrogenated at 1 atmosphere with a hydrogen-filled balloon. The reaction mixture was filtered over celite, concentrated in vacuo, and used directly in the next step. This phenol intermediate (647 mg, 3.9 mmol) was diluted into methylene chloride (5 mL), and treated with triethylamine (1.63 mL, 11.7 mmol), followed by trifluoromethanesulfonic anhydride (1.97 mL, 11.7 mmol). Upon reaction completion, the reaction mixture was concentrated in vacuo, and the triflate was purified via preparative RPHPLC. This triflate methyl ester (100 mg, 0.34 mmol) was combined with 1-naphthylboronic acid (572 mg, 3.4 mmol), 10% catalytic tetrakis-(triphenylphosphine)palladium, and 10 equivalents of potassium carbonate, diluted in (3:1) toluene-water (7 mL). The reaction mixture was refluxed overnight in a sealed tube, cooled to room temperature, concentrated in vacuo, partitioned between water and methylene chloride, the organic phase separated, concentrated in vacuo, and the residue purified via preparative RPHPLC. The methyl ester was saponified with LiOH in a manner similar to EXAMPLE 2, and the resultant acetic acid intermediate (0.74 mmol) was combined with HOAt (1.5 equiv, 151 mg, 1.11 mmol), EDCI (1.5 equiv, 212 mg, 1.11 mmol), and benzyl anthranilate (1.5 equiv, 252 mg, 1.11 mmol) in methylene chloride. Upon standard extractive work-up, the crude coupled amide benzyl ester was hydrogenated with catalytic palladium on carbon in ethyl acetate solvent under conditions described in the examples above, and the crude purified via preparative RPHPLC to give the desired product acid: ¹H NMR (CDCl₃, 500 MHz) δ 10.8 (s, 1H), 8.8 (d, 1H), 7.95 (d, 2H), 7.9 (d, 1H), 7.8 (d, 1H), 7.6 (t, 1H), 7.5 (m, 6H) 7.4 (t, 1H), 7.1 (t, 1H); LCMS m/z 382 (M⁺+1).

### Example 5

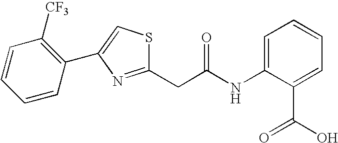

Commercially available ethyl (4-hydroxy-thiazol-2-yl)acetate (250 mg, 1.33 mmol) was diluted into methylene chloride (5 mL), and treated with triethylamine (556 uL, 4.0 mmol), followed by the addition of trifluoromethanesulfonic anhydride (676 uL, 4.0 nmmol) at 0° C. The reaction mixture was warmed to room temperature for 1 h, partitioned between water and methylene chloride, the organic phase separated, concentrated in vacuo, and the triflate was purified via preparative RPIHPLC. This triflate (50 mg, 0.16 mmol) was coupled with 2-(trifluoromethyl)phenylboronic acid under Suzuki conditions described in EXAMPLE 4 above. The ethyl ester was saponified with LiOH in a manner similar to EXAMPLE 2 and used directly in the next step. This acid intermediate (23 mg, 0.08 mmol) was diluted into tetrahydrofuran (2 mL), and treated with triethylamine (45 uL, 0.32 mmol), followed by 2,4,6-trichlorobenzoyl chloride (25 uL, 0.16 mmol) and benzyl anthranilate (18 mg, 0.08 mmol). Upon reaction completion, the reaction mixture was concentrated in vacuo, and the benzyl ester was saponified with LiOH in a manner similar to EXAMPLE 2. The crude was purified via preparative RPHPLC to give the desired product acid: ¹H NMR (DMSO-d₆, 500 MHz) δ 8.4 (d, 1H), 8.0 (d, 1H), 7.9 (d, 2H), 7.7 (m, 3H), 7.6 (m, 2H), 7.2 (t, 1H), 4.3 (s, 2H); LCMS m/z 407 (M⁺+1).

### Example 6

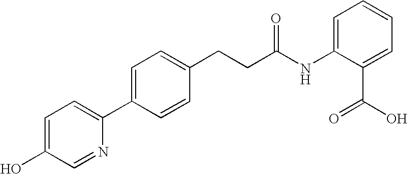

Commercially available 4-(2-carboxyethyl)benzeneboronic acid (194 mg, 1.0 mmol) was coupled with commercially available 2-bromo-5-nitropyridine (203 mg, 1.0 mmol) under similar Suzuki conditions described for EXAMPLE 1. The product acid (109 mg, 0.28 mmol) was converted to its acid chloride and subsequent anthranilide in a manner similar to EXAMPLE 1. This nitro intermediate (48 mg, 0.095 mmol) was reduced with SnCl₂ (60 mg, 0.32 mmol) in ethanol (10 mL) for 3 h at room temperature, then heated at reflux for 14 h. The reaction mixture was then cooled to room temperature, concentrated in vacuo, and purified via preparative RPHPLC to give the amine intermediate. This amine TFA-salt (25 mg, 0.053 mmol) was diluted into 2M aqueous sulfuric acid (5 mL), cooled to 0° C., and treated slowly with NaNO₂ (7 mg, 0.106 mmol). The slurry was warmed to room temperature, stirred overnight, then heated at 100° C. for 10 min, the resultant clear solution was concentrated in vacuo, and the crude was purified via preparative RPHPLC to give the desired product acid: ¹H NMR (acetone-d₆, 500 MHz) δ 11.2 (s, 1H), 8.74 (d, 1H), 8.43 (d, 1H), 8.09 (d, 1H), 7.92 (t, 3H), 7.60 (m, 2H), 7.44 (d, 2H), 7.15 (t, 1H), 3.11 (t, 2H), 2.82 (t, 2H); LCMS m/z 363 (M⁺+1).

### Example 7

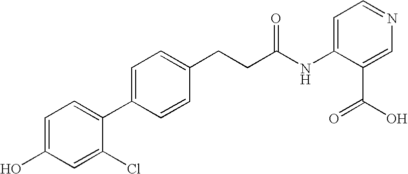

Commercially available 4-chloronicotinic acid (1 g, 6.36 mmol) was combined with 30% ammonium hydroxide (20 mL) in an autoclave, and the reaction mixture was heated at 180° C. for 6 h. The mixture was cooled to room temperature, concentrated until a light yellow solid precipitated from solution, and then the 4-aminonicotinic acid product was filtered pure. This 4-aminonicotinic acid was coupled under similar SOCl₂ conditions described in EXAMPLE 1, with the methoxychlorobiphenyl acid shown in Scheme 6, itself prepared under similar Suzuki conditions also described in EXAMPLE 1. The resultant amidobiaryl methyl ether was demethylated with BBr₃ under similar conditions described in EXAMPLE 3, and the crude was purified via preparative RPHPLC to give the desired product: ¹H NMR (DMSO-d₆, 500 MHz) δ 11.9 (s, 1H), 9.19 (s, 1H), 8.81 (d, 1H), 8.76 (d, 1H), 7.31 (d, 2H), 7.28 (d, 2H), 7.16 (d, 1H), 6.89 (d, 1H), 6.79 (dd, 1H), 2.98 (br.m, 4H); LCMS m/z 397 (M⁺+1).

### Example 8

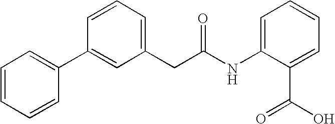

EXAMPLE 8 was prepared under similar conditions described in EXAMPLE 4, and purified via preparative RPHPLC to give the desired product: ¹H NMR (CDCl₃, 500 MHz) δ 10.8 (s, 1H), 8.8 (d, 1H), 8.3 (d, 1H), 7.8 (t, 1H), 7.3 (t, 1H), 7.0 (m, 3H), 6.1 (s, 2H) 3.2 (t, 2H), 2.9 (t, 2H); LCMS m/z 332 (M⁺+1).

### Example 9

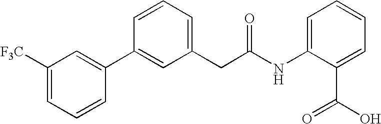

EXAMPLE 9 was prepared under simnilar conditions described in EXAMPLE 4, and purified via preparative RPHPLC to give the desired product: ¹H NMR (CDCl₃, 500 MHz) δ 10.9 (s, 1H), 8.9 (d, 1H), 7.95 (d, 1H), 7.9 (s, 1H), 7.8 (d, 1H), 7.6 (m, 4H), 7.4 (d, 1H), 7.1 (t, 1H), 3.9 (s, 2H); LCMS m/z 400 (M⁺+1).

### Example 10

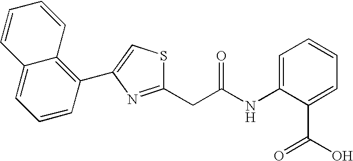

EXAMPLE 10 was prepared under similar conditions described in EXAMPLE 5, and purified via preparative RPHPLC to give the desired product: ¹H NMR (CD₂Cl₂, 500 MHz) δ 11.8 (s, 1H), 8.9 (d, 1H), 8.3 (d, 1H), 8.0 (m, 3H), 7.6 (d, 1H), 7.5 (m, 5H), 7.1 (t, 1H), 4.6 (s, 2H); LCMS m/z 389 (M⁺+1).

### Example 11

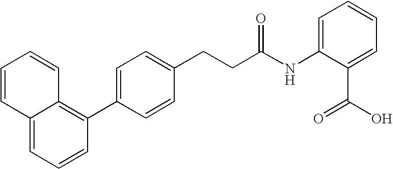

EXAMPLE 11 was prepared under similar conditions described in EXAMPLE 1, except that commercially available 3-(4-bromophenyl)propionic acid was first coupled with anthranilic acid under the same SOCl₂ conditions described, and this bromo anthranilide carboxylate (50 mg, 0.144 mmol) was then coupled directly with the boronic acid. The crude was purified via preparative RPHPLC (Gilson) to give the desired product: ¹H NMR (acetone-d₆, 500 MHz) δ 11.30 (1H, s), 8.80 (1H, d), 8.13 (1H, q), 7.98(3H, m), 7.64-7.41(9H, m), 7.17(1H, m), 3.17(2H, t), 2.87(2H, t); LCMS m/z 394 (M⁺−1).

### Example 12

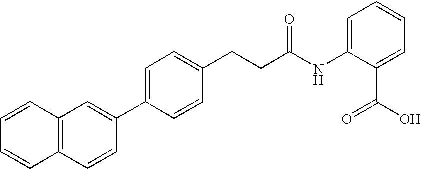

EXAMPLE 12 was prepared in the same manner as EXAMPLE 11, and purified via preparative RPHPLC (Gilson) to give the desired product: ¹H NMR (DMSO-d₆, 500 MHz) δ 11.19(1H, s), 8.48(1H, d), 8.17-7.40(13H, m), 7.13(1H, s), 2.77(2H, t), 2.49(2H, t); LCMS m/z 394 (M⁺−1).

### Example 13

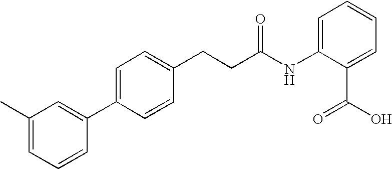

EXAMPLE 13 was prepared in the same manner as EXAMPLE 11, and purified via preparative RPHPLC (Gilson) to give the desired product: ¹H NMR (acetone-d₆, 500 MHz) δ 11.28(1H, s), 8.78 (1H, q), 8.11(1H, q), 7.60(3H, m), 7.40(4H, m), 7.32(1H, t), 7.15(2H, m), 3.10(2H, t), 2.82(2H, t), 2.39(3H, s); LCMS m/z 358 (M⁺−1).

### Example 14

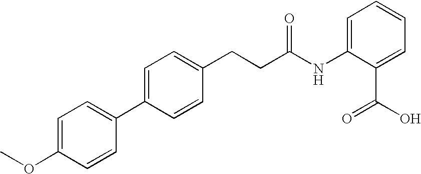

EXAMPLE 14 was prepared in the same manner as EXAMPLE 11, and purified via preparative RPHPLC (Gilson) to give the desired product: ¹H NMR (DMSO-d₆, 500 MHz) δ 11.18(1H, s), 8.48(1H, d), 7.96(1H, q), 7.56(5H, m), 7.32(2H, d), 7.14(1H, t), 6.99(2H, t), 3.77(3H, s), 2.98(2H, t), 2.75(2H, t); LCMS m/z 374 (M⁺−1).

### Example 15

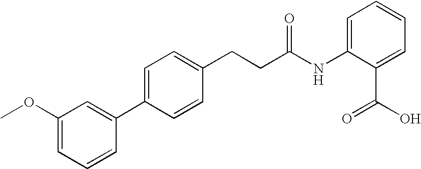

EXAMPLE 15 was prepared in the same manner as EXAMPLE 11, and purified via preparative RPHPLC (Gilson) to give the desired product: ¹H NMR (DMSO-d₆, 500 MHz) δ 11.15(1H, s), 8.48(1H, d), 7.97(1H, d), 7.57(3H, m), 7.33(3H, m), 7.19(3H, m), 7.90(1H, d), 3.79(3H, s), 2.98(2H, t), 2.76(2H, t); LCMS m/z 374 (M⁺−1).

### Example 16

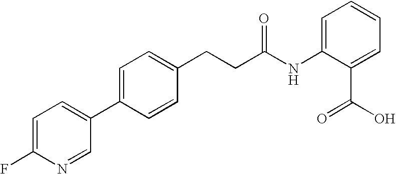

EXAMPLE 16 was prepared in the same manner as EXAMPLE 11, and purified via preparative RPHPLC (Gilson) to give the desired product: ¹H NMR (acetone-d₆, 500 MHz) δ 11.30(1H, s), (8.76(1H, d), 8.43(1H, s), 8.20(1H, m), 8.11(1H, q), 7.62(3H, m), 7.451(2H, d), 7.17(2H, m), 3.04(2H, t), 2.86(2H, t); LCMS m/z 363 (M⁺−1).

### Example 17

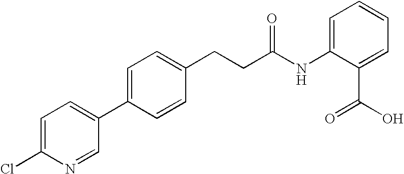

EXAMPLE 17 was prepared in the same manner as EXAMPLE 11, and purified via preparative RPHPLC (Gilson) to give the desired product: ¹H NMR (acetone-d₆, 500 MHz) δ 11.26 (1H, s), (8.76(1H, d), 8.43(1H, d), 8.11(1H, q), 7.76(2H, d), 7.721(1H, s), 7.67(1H, d), 7.62(1H, t), 7.50(2H, d), 7.17(1H, t), 3.04(2H, t), 2.86(2H, t); LCMS m/z 381 (M⁺+1).

### Example 18

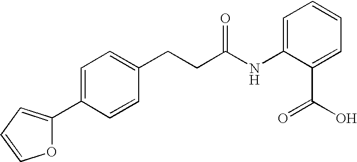

EXAMPLE 18 was prepared in the same manner as EXAMPLE 11, and purified via preparative RPHPLC (Gilson) to give the desired product: ¹H NMR (CD₃OD, 500 MHz) δ 8.55(1H, d), 8.07(1H, q), 7.55(4H, m), 7.29(2H, d), 7.13(1H, m), 6.68(1H, d), 6.48(1H, q), 3.06(2H, t), 2.77(2H, t); LCMS m/z 334 (M⁺−1).

### Example 19

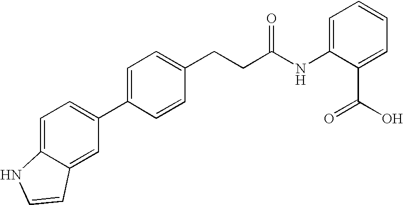

EXAMPLE 19 was prepared in the same manner as EXAMPLE 11, and purified via preparative RPHPLC (Gilson) to give the desired product: ¹H NMR (acetone-d₆, 500 MHz) δ 11.1 (s, 1H), 10.3 (s, 1H), 8.77 (d, 1H), 8.10 (d, 1H), 7.83 (s, 1H), 7.60 (d, 2H), 7.49 (d, 1H), 7.39 (m, 5H), 7.15 (t, 1H), 6.53 (s, 1H), 3.09 (t, 2H), 2.81 (t, 2H); LCMS m/z 383 (M⁺−1).

### Example 20

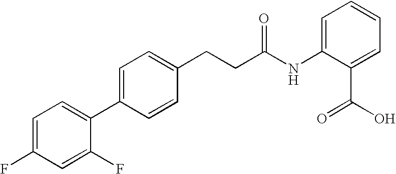

EXAMPLE 20 was prepared in the same manner as EXAMPLE 11, and purified via preparative RPHPLC (Gilson) to give the desired product: ¹H NMR (acetone-d₆, 500 MHz) δ 11.3 (s, 1H), 8.76 (d, 1H), 8.10 (dd, 1H), 7.50 (m, 6H), 7.11 (m, 3H), 3.11 (t, 2H), 2.82 (t, 2H); LCMS m/z 380 (M⁺−1)

### Example 21

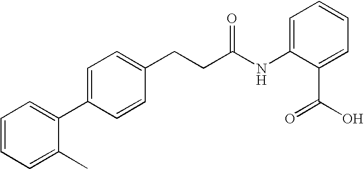

EXAMPLE 21 was prepared in the same manner as EXAMPLE 11, and purified via preparative RPHPLC (Gilson) to give the desired product: ¹H NMR (acetone-d₆, 500 MHz) δ 11.3 (s, 1H), 8.78 (dd, 1H), 8.10 (dd, 1H), 7.61 (m, 1H), 7.38 (d, 2H), 7.23 (m, 7H), 3.11 (t, 2H), 2.82 (t, 2H), 2.23 (s, 3H); LCMS m/z 360 (M⁺+1).

### Example 22

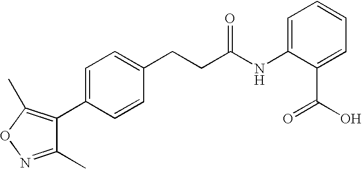

EXAMPLE 22 was prepared in the same manner as EXAMPLE 11, and purified via preparative RPHPLC (Gilson) to give the desired product: ¹H NMR (acetone-d₆, 500 MHz) δ 11.3 (s, 1H), 8.77 (d, 1H), 8.12 (dd, 1H), 7.62 (m, 1H), 7.44 (d, 2H), 7.31 (d, 2H), 7.17 (t, 1H), 3.10 (t, 2H), 2.83 (t, 2H), 2.40 (s, 3H), 2.23 (s, 3H); LCMS m/z 348 (M⁺+1).

### Example 23

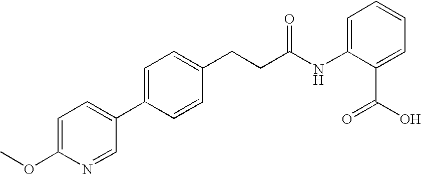

EXAMPLE 23 was prepared in the same manner as EXAMPLE 11, and purified via preparative RPHPLC (Gilson) to give the desired product: ¹H NMR (DMSO-d₆, 500 MHz) δ 11.1 (s, 1H), 8.47 (d, 1H), 8.44 (d, 1H), 7.96 (m, 1H), 7.56 (m, 3H), 7.35 (d, 2H), 7.13 (t, 1H), 6.88 (d, 1H), 3.87 (s, 3H), 2.98 (t, 2H), 2.75 (t, 2H); LCMS m/z 377 (M⁺+1).

### Example 24

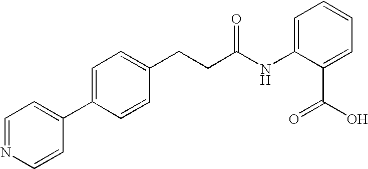

EXAMPLE 24 was prepared in the same manner as EXAMPLE 11, and purified via preparative RPHPLC (Gilson) to give the desired product (21 mg): ¹H NMR (DMSO-d₆, 500 MHz) δ 11.1 (s, 1H), 8.77 (d, 2H), 8.46 (d, 1H), 8.06 (d, 2H), 7.95 (d, 1H), 7.86 (d, 2H), 7.57 (t, 1H), 7.48 (d, 2H), 3.03 (t, 2H), 2.79 (t, 2H); LCMS m/z 347 (M⁺+1).

### Example 25

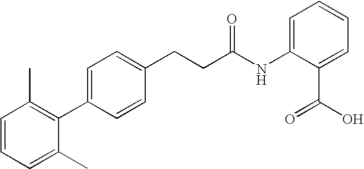

EXAMPLE 25 was prepared in the same manner as EXAMPLE 11, and purified via preparative RPHPLC (Gilson) to give the desired product: ¹H NMR (acetone-d₆, 500 MHz) δ 11.3 (s, 1H), 8.77 (d, 11H), 8.10 (d, 11H), 7.60 (m, 11H), 7.39 (d, 2H), 7.13 (m, 6H), 3.11 (t, 2H), 2.82 (t, 2H), 1.96 (s, 6H); LCMS m/z 372 (M⁺−1).

### Example 26

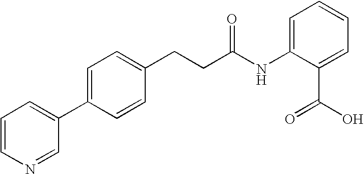

EXAMPLE 26 was prepared in the same manner as EXAMPLE 11, and purified via preparative RPHPLC (Gilson) to give the desired product: ¹H NMR (DMSO-d₆, 500 MHz) δ 11.1 (s, 1H), 9.04 (s, 1H), 8.70 (d, 1H), 8.46 (t, 2H), 7.96 (dd, 1H), 7.78 (m, 1H), 7.72 (d, 2H), 7.57 (m, 1H), 7.44 (d, 2H), 7.13 (t, 1H), 3.02 (t, 2H), 2.78 (t, 2H); LCMS m/z 347 (M⁺+1).

### Example 27

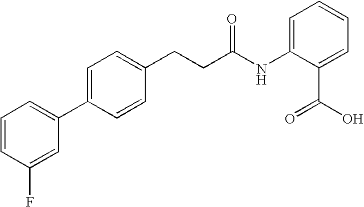

EXAMPLE 27 was prepared in the same manner as EXAMPLE 11, and purified via preparative RPHPLC (Gilson) to give the desired product: ¹H NMR (acetone-d₆, 500 MHz) δ 11.3 (s, 1H), 8.77 (d, 1H), 8.10 (dd, 1H), 7.61 (m, 3H), 7.44 (m, 5H), 7.11 (m, 2H), 3.11 (t, 2H), 2.82 (t, 2H); LCMS m/z 362 (M⁺−1).

### Example 28

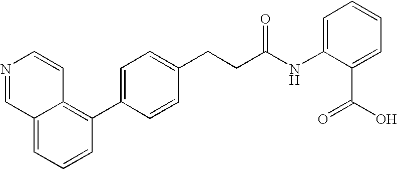

EXAMPLE 28 was prepared in the same manner as EXAMPLE 11, and purified via preparative RPHPLC (Gilson) to give the desired product: ¹H NMR (CD₃OD, 500 MHz) δ 9.68 (s, 1H), 8.57 (d, 1H), 8.45 (bs, 1H), 8.39 (d, 1H), 8.13 (s, 1H), 8.04 (m, 3H), 7.56 (t, 1H), 7.52 (d, 2H), 7.47 (d, 2H), 7.14 (t, 1H), 3.18 (t, 2H), 2.85 (t, 2H); LCMS m/z 397 (M⁺+1).

### Example 29

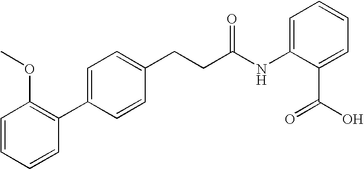

EXAMPLE 29 was prepared in the same manner as EXAMPLE 11, and purified via preparative RPHPLC (Gilson) to give the desired product: ¹H NMR (DMSO-d₆, 500 MHz) δ 11.2 (s, 1H), 8.49 (d, 1H), 7.98 (d, 1H), 7.57 (m, 2H), 7.28 (m, 7H), 7.01 (t, 1H), 3.73 (s, 3H), 2.96 (t, 2H), 2.76 (t, 2H); LCMS m/z 374 (M⁺−1).

### Example 30

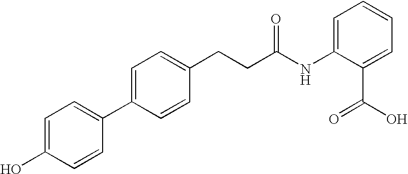

EXAMPLE 30 was prepared in the same manner as EXAMPLE 11, and purified via preparative RPHPLC (Gilson) to give the desired product: ¹H NMR (acetone-d₆, 500 MHz) δ 11.4 (s, 1H), 8.67 (d, 1H), 8.05 (d, 1H), 7.58 (t, 1H), 7.48 (d, 2H), 7.44 (d, 2H), 7.34 (d, 2H), 7.14 (t, 1H), 6.89 (d, 1H), 3.06 (t, 2H), 2.79 (t, 2H); LCMS m/z 360 (M⁺−1).

### Example 31

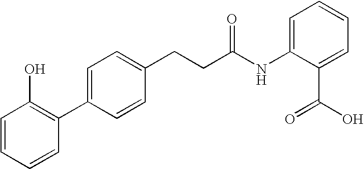

EXAMPLE 31 was prepared from EXAMPLE 29 (10 mg, 0.027 mmol) under similar demethylation conditions described in EXAMPLE 3. The crude was purified via preparative RPHPLC (Gilson) to give the desired product: ¹H NMR (acetone-d₆, 500 MHz) δ 11.3 (s, 1H), 8.78 (d, 1H), 8.12 (d, 1H), 7.62 (t, 1H), 7.54 (d, 2H), 7.36 (d, 2H), 7.29 (d, 2H), 7.15 (q, 1H), 6.99 (d, 1H), 6.93 (t, 1H), 3.10 (t, 2H), 2.83 (t, 2H).

### Example 32

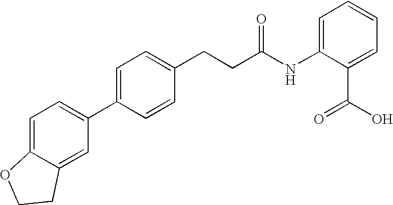

EXAMPLE 32 was prepared in the same manner as EXAMPLE 11, and purified via preparative RPHPLC (Gilson) to give the desired product: ¹H NMR (acetone-d₆, 500 MHz) δ 11.3 (s, 1H), 8.78 (d, 1H), 8.12 (d, 1H), 7.63 (t, 1H), 7.51 (m, 3H), 7.37 (d, 2H), 7.17 (t, 1H), 6.80 (d, 1H), 4.60 (t, 2H), 3.28 (t, 2H), 3.09 (t, 2H), 2.81 (t, 2H); LCMS m/z 386 (M⁺−1).

### Example 33

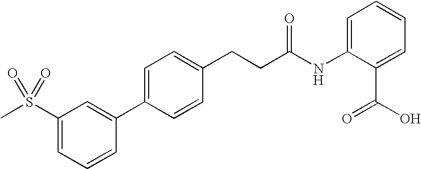

EXAMPLE 33 was prepared in the same manner as EXAMPLE 11, and purified via preparative RPHPLC (Gilson) to give the desired product: ¹H NMR (acetone-d₆, 500 MHz) δ 11.3 (s, 1H), 8.76 (d, 1H), 8.18 (m, 1H), 8.15 (dd, 1H), 7.99 (m, 1H), 7.92 (m, 1H), 7.75 (m, 1H), 7.68 (m, 2H), 7.59 (m, 1H), 7.46 (d, 2H), 7.15 (t, 1H), 3.20 (s, 3H), 3.12 (t, 2H), 2.82 (t, 2H); LCMS m/z 422 (M⁺−1).

### Example 34

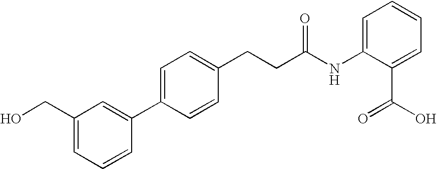

EXAMPLE 34 was prepared in the same manner as EXAMPLE 11, and purified via preparative RPHPLC (Gilson) to give the desired product: ¹H NMR (acetone-d₆, 500 MHz) δ 11.3 (s, 1H), 8.77 (d, 1H), 8.10 (dd, 1H), 7.70-7.28 (m, 10H), 7.15 (t, 1H), 4.71 (d, 2H), 3.10 (t, 2H), 2.81 (t, 2H); LCMS m/z 374 (M⁺−1).

### Example 35

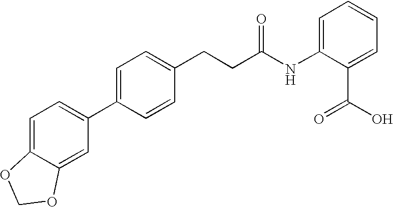

EXAMPLE 35 was prepared in the same manner as EXAMPLE 11, and purified via preparative RPHPLC (Gilson) to give the desired product: ¹H NMR (acetone-d₆, 500 MHz) δ 11.3 (s, 1H), 8.76 (dd, 1H), 8.10 (dd, 1H), 7.61 (m, 1H), 7.50 (dd, 2H), 7.36 (d, 2H), 7.13 (m, 3H), 6.92 (t, 1H), 6.03 (s, 2H), 3.08 (t, 2H), 2.82 (t, 2H); LCMS m/z 388 (M⁺−1).

### Example 36

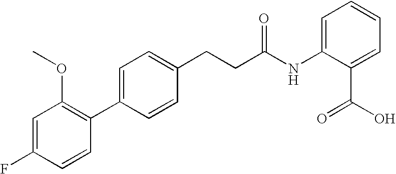

EXAMPLE 36 was prepared in the same manner as EXAMPLE 11, and purified via preparative RPHPLC (Gilson) to give the desired product: ¹H NMR (acetone-d₆, 500 MHz) δ 11.3 (s, 1H), 8.75 (d, 1H), 8.08 (dd, 1H), 7.59 (m, 1H), 7.40 (d, 2H), 7.38 (d, 2H), 7.28 (dd, 1H), 7.15 (t, 1H), 6.88 (dd, 1H), 6.77 (td, 1H), 3.81 (s, 3H), 3.08 (t, 2H), 2.80 (t, 2H); LCMS m/z 392 (M⁺−1).

### Example 37

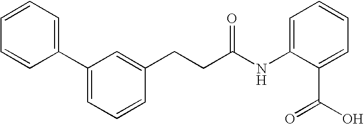

EXAMPLE 37 was prepared under similar conditions described in EXAMPLE 1, except that commercially available 3-(3-iodophenyl)propionic acid was used instead. The crude was purified via preparative RPHPLC (Gilson) to give the desired product: ¹H NMR (acetone-d₆, 500 MHz) δ 11.30(1H, s), 8.79(1H, d), 8.12(1H, m), 7.66-7.60(4H, m), 7.50-7.32(6H, m), 7.18(1H, m), 3.14(2H, t), 2.85(2H, t); LCMS m/z 346 (M⁺+1).

### Example 38

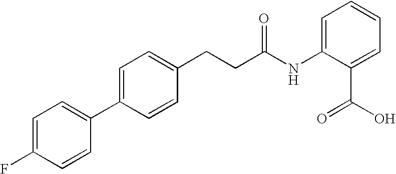

EXAMPLE 38 was prepared in the same manner as EXAMPLE 11, and purified via preparative RPHPLC (Gilson) to give the desired product: ¹H NMR (DMSO-d₆, 500 MHz) δ 11.42(1H, s), 8.48(1H, d), 7.96(1H, d), 7.65-7.12(10H, m), 2.97(2H, t), 2.74(2H, t); LCMS m/z 362 (M⁺−1).

### Example 39

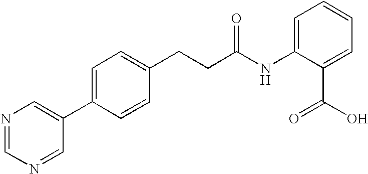

EXAMPLE 39 was prepared in the same manner as EXAMPLE 11, and purified via preparative RPHPLC (Gilson) to give the desired product: ¹H NMR (DMSO-d₆, 500 MHz) δ 11.40(1H, s), 9.14(3H, m), 8.47(1H, d), 7.96(1H, d), 7.72(2H, d), 7.58(1H, t), 7.43(2H, d), 7.12(1H, t), 3.00(2H, t), 2.78(2H, t); LCMS m/z 346 (M⁺−1).

### Example 40

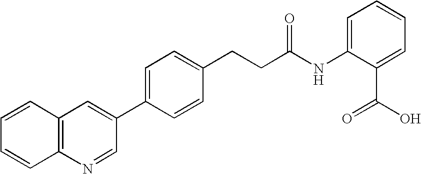

EXAMPLE 40 was prepared in the same manner as EXAMPLE 11, and purified via preparative RPHPLC (Gilson) to give the desired product: ¹H NMR (DMSO-d₆, 500 MHz) δ 11.45(1H, s), 9.32(1H, s), 8.807(1H,s), 8.49(1H, d), 8.10(2H, t), 7.96(1H, d), 7.74(3H, m), 7.70(1H, m), 7.57(1H, m), 7.47(2H, m), 7.14(1H, m), 3.03(2H, t), 2.80(2H, t); LCMS m/z 395 (M⁺−1).

### Example 41

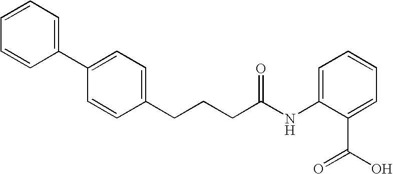

EXAMPLE 41 was prepared under similar conditions described in EXAMPLE 1, except that commercially available 4-(para-iodophenyl)butyric acid was used instead. The crude was purified via preparative RPHPLC (Gilson) to give the desired product: ¹H NMR (DMSO-d₆, 500 MHz) δ 11.13 (1H, s), 8.48(1H, d), 7.97(1H, d), 7.63(2H, d), 7.58(3H, m), 7.45(2H, t), 7.34(3H, m), 7.13 (1H, t), 2.67(2H, t), 2.49(2H, t), 1.95(2H, m); LCMS m/z 360 (M⁺+1).

### Example 42

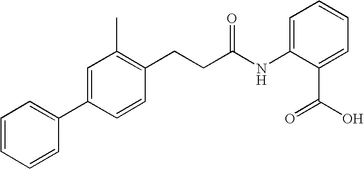

A mixture of 4-bromo-2-methyl-benzoic acid (430 mg), phenyl boronic acid (317 mg), sodium bicarbonate (4 mL, 1 M), dioxane (20 mL) and palladium tetrakistriphenylphosphine (50 mg) was heated at 100° C. for 12 hours. The mixture was filtered through celite and directly purified from RP-HPLC (Varian) to give 4-phenyl-2-methyl-benzoic acid as a light yellow solid. To 4-phenyl-2-methyl-benzoic acid (363 mg) was added TIF (15 mL). The mixture was cooled to 0° C. To this mixture was then added lithium aluminum hydride (130 mg). The mixture was slowly warmed to RT and stirred for 12 hours. The mixture was cooled to 0° C. again and quenched with the aqueous solution of Rochelle's salt. Extracted the mixture with ethyl acetate, dried the organic layer with sodium sulfate and concentrated it in vacuo. The resulting light yellow oil was the desired 4-phenyl-2-methyl-benzyl alcohol. To 4-phenyl-2-methyl-benzyl alcohol (188 mg) was added 4A molecular sieves, methylene chloride (10 mL) and pyridinium chlorochromate (410 mg). After 2 hours, the crude mixture was directly purified by biotage silica gel column (5% to 15% ethyl acetate in hexane) to give 4-phenyl-2-methyl-benzaldehyde as a light yellow oil. To a solution of trimethyl phosphonate acetate (176 mg) in 5 mL of THF was added n-butyl]ithium (0.69 mL, 1.6 M in hexane) at 0° C. The resulting solution was stirred at this temperature for 30 min. To this solution was added a THF solution (5 mL) of 4-phenyl-2-methyl-benzaldehyde (135 mg). The mixture was slowly warmed to rt and stirred for 2 hours. After quenching the mixture was water, the mixture was extracted with ethyl acetate, dried with sodium sulfate and concentrated in vacuo to give 2-methyl-4-phenyl-1-(methyl-1-acrylate) as a yellow oil. To 2-methyl-4-phenyl-1-(methyl-1-acrylate) (177 mg) was added 5 mL of THF:MeOH:water (3:1:1) followed by LiOH (5 mL, 1 M). The mixture was stirred at rt for 8 hours. After acidified with concentrated HCl until pH=3, the slurry was extracted with 30% isopropanol in chloroform, dried with sodium sulfate and concentrated in vacuo to give 2-methyl-4-phenyl-1-(1-acrylic acid) as a white solid. To 2-methyl-4-phenyl-1-(1-acrylic acid) (129 mg) was added toluene (5 mL) and thionyl chloride (2 mL). The mixture was heated to reflux for 2 hours and the solvent was distilled off under reduced pressure. The residue was taken up with toluene (5 mL) and to it was added anthranilic acid (111 mg). The resulting mixture was heated to reflux for additional 2 hours. The solvent was removed and the residue was taken up with DMSO and purified by RPHPLC (Gilson) to give the desired amide as an off-white solid. To the above amide (26 mg) was added methanol and Pd/C (5 mg, 10%). Under 1 atm of hydrogen balloon, the mixture was stirred for 2 hours. The mixture was filtered with celite, the filtrate was concentrated in vacuo to give Example 42 as an off-white solid. ¹H NMR (acetone-d₆, 500 MHz) δ 11.4(1H, s), 8.77(1H, d), 8.10(1H, d), 7.62(1H, m), 7.43(5H, m), 7.14(1H, bs), 7.21(1H, d), 7.18(1H, d), 7.15(1H, t), 3.09(2H, t), 2.76(2H, t), 2.45(3H, s); LCMS m/z 358 (M−1), 360 (M⁺+1).

### Example 43

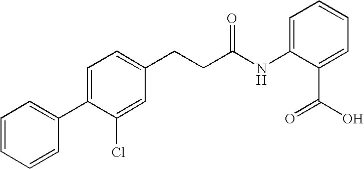

Following the same reaction sequence as the preparation of Example 42, the desired product was obtained as a crystalline solid. ¹H NMR (acetone-d₆, 500 MHz) δ 11.3(1H, s), 8.76(1H, d), 8.11(1H, dd), 7.61(1H, m), 7.51(1H, d), 7.44(4H, m), 7.40(2H, m), 7.32(1H, d), 7.16(1H, t), 3.11(2H, t), 2.85(2H, t); LCMS m/z 378 (M−1), 380 (M⁺+1).

### Example 44

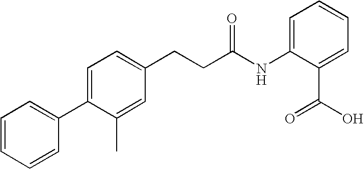

The same procedure described in the preparation of Example 42 gave the desired product as a white solid. ¹HNMR (acetone-d₆, 500 MHz) δ 11.3(1H, s), 8.79(1H, d), 8.11(1H, d), 7.61(1H, m), 7.40(2H, m), 7.35(2H, m), 7.18(5H, m), 3.05(2H, t), 2.82(2H, t), 2.21(3H, s); LCMS m/z 358 (M−1), 360 (M⁺+1).

### Example 45

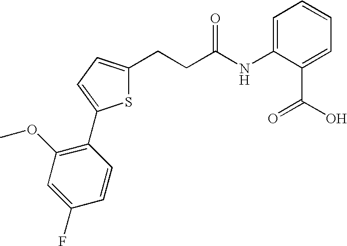

To a solution of 5-bromothiophene-2-carboxaldehyde (5.85 g, 30.6 mmol) in anhydrous THF (150 mL) which was cooled by ice-bath, was added DIBAL (36.7 mL, 1N in toluene) dropwisely over 15 min. The resulting was stirred at RT for 2 hours. The reaction was quenched by adding sat. potassium tartrate. The mixture was extracted with EtOAc, the organic phase was washed with brine, dried over Na₂SO₄. The solvent was evaporated on rotary evaporation to obtain a brown oil. To a solution of this alcohol (5.80 g, 30 mmol) in methylene chloride (100 mL), at 0° C., was CBr₄ (14.92 g, 45 mmol) in one portion. To the resulting solution was added a solution of PPh₃ (11.8 g, 45 mmol) in CH₂Cl₂ (20 mL) dropwisely, after the mixture was stirred at r.t. for 2 h, the solvent was evaporated and the residue was purified by silica gel chromatography using hexane as eluting solvent to obtain the bromide as an oil. To a solution of dimethylmalonate (1.50 mL, d=1.156, 13.1 mmol) in THF (100 mL), at 0° C., was added NaH (0.364 g, 95%). After stirring at 0° C. for 10 mins, to the resulting mixture was added a solution of the bromide (3.36 g, 13.1 mmol) in THF(30 mL) dropwise, after stirring at RT for 4 h, the mixture was filtered and the filtrate was concentrated and purified on silica gel chromatography using 5% EtOAc/Hexane as eluting solvent to obtained the product. A solution of this dimethyl ester intermediate (0.82 g, 2.6 mmol) in 20 mL of THF/MeOH/H₂O (3:1:1) was treated with 10 mL 1 N LiOH and stirred at r.t. overnight. After removed the organic solvent, the aqueous solution was acidified to pH 3, and extracted with EtOAc, the organic phase was washed with brine and dried over Na₂SO₄. Concentration of the solution gave a brown solid. This diacid in DMF (4 mL) was heated in Microwave at 170° C. for 2 mins. The mixture was partitioned between EtOAc and water, the organic phase was washed with brine and dried over Na₂SO₄. After removed the solvent, the residue was purified on silica gel using 5% MeOH/DCM to obtain a brown solid. A solution of this acid intermediate (0.54 g, 2.297 mmol) in 20 mL anhydrous toluene was treated with 3 mL thionyl chloride, and heated at 100° C. for 45 mins. The solvent was removed by distillation and the residue was treated with methyl anthranilate in 20 mL toluene, the resulting mixture was heated to reflux for 1 h. The solvent was evaporated on rotary evaporator and residue was dissolved in 50 mL EtOAc, insoluble solid was filtered and the filtrate was washed with 3N HCl (3×30 mL) and brine, dried over Na₂SO₄, concentration of the solution gave the product. A solution of this anthranilide methyl ester (0.83 g, 2.254 mmol) in 40 mL of THF/MeOH/H₂O (3:1:1) was treated with 10 mL 1N LiOH and stirred at r.t. for 1 h. After removed the organic solvent, the aqueous solution was acidified to pH 3, and extracted with EtOAc, the organic phase was washed with brine and dried over Na₂SO₄. Concentration of the solution gave the brown solid acid. A mixture of 2-methoxy-4-fluorophenylboronic acid (7.5 mg, 0.0439 mmol), the bromo anthranilide acid (12 mg, 0.0338 mmol), catalytic amount of Ph(PPh₃)₄, sodium bicarbonate (1N, 0.14 mL) in dioxane (4 mL) was heated at 100° C. under argon overnight. The reaction mixture was filtered and the filtrate was purified by RP-HPLC (Gilson) to obtain Example 45. ¹H NMR (DMSO-d₆, 500 MHz) δ 11.14 (1H, s), 8.47(1H, d), 7.97(1H, d), 7.63(2H, m), 7.30(1H, d), 7.15(1H, t), 7.02(1H, m), 6.87(1H, d), 6.79(1H, m), 3.85(3H, s), 3.16(2H, t), 2,79(3H, t); LCMS m/z 398.36 (M⁺−1), 400.30 (M⁺+1), 422.29(M⁺+23).

### Example 46

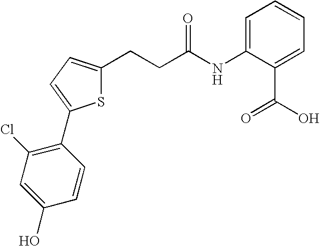

Example 46 was prepared under similar conditions described in Example 45, except that commercially available 2-chloro-4-methoxyphenylboronic acid was used instead. The crude was purified via preparative RPHPLC (Gilson) to give the desired product methyl ether. To a solution of the methyl ether (14 mg, 0.0336 mmol) in 10 mL CH₂Cl₂, at 0° C., was added BBr₃ (0.1344 mL, 1N in CH₂CL₂) dropwisely, After stirring at r.t. for 6 h, the reaction was quenched by water at 0° C., the CH₂Cl₂ phase was washed with brine and concentrated. The resulting residue was purified on preparative RPHPLC (Gilson) to give Example 46. ¹H NMR (acetone-d₆, 500 MHz) δ 11.32 (1H, s) 8.79(1H, d), 8.13(1H, d), 7.64(1H, t), 7.41(1H, d), 7.18(1H, t), 7.10(1H, d), 7.00(1H, d), 6.96(1H, d), 6.88(1H, m),3.29(2H, t), 2.88(2H, t); LCMS m/z 402.24( M⁺+1), 400.33 (M⁺−1).

### Example 47

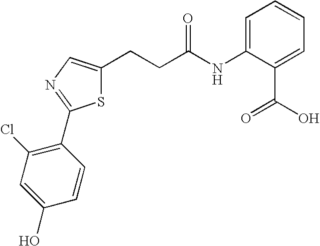

The mixture of 2-chloro-4-methoxyphenyl boronic acid (372 mg), 2-bromo-5-formylthiazole(576 mg), sodium bicarbonate (6 mL, 1 M), dioxane (6 mL) and palladium tetrakistriphenylphosphine (30 mg) was heated at 100° C. for 4 hours. The mixture was filtered through celite and diluted with ethyl acetate (100 mL) and washed with water (100 mL) followed by brine (50 mL). The organic fraction was dried with sodium sulfate and concentrated in vacuo to give the coupled product as a brown solid. To a solution of trimethyl phosphonoacetate (146 mg) in 5 mL of THF was added n-butyl]ithium (0.59 mL, 1.6 M in hexane) at 0° C. The resulting solution was stirred at this temperature for 30 min. To this solution was added a THF solution (5 mL) of the above intermediate aldehyde (170 mg). The mixture was slowly warmed to rt and stirred for 2 hours. After quenching the mixture was water, the mixture was extracted with ethyl acetate, dried with sodium sulfate and concentrated in vacuo to give the enoate as a brown oily solid. To this enoate (83 mg) was added 5 mL of THF:MeOH:water (3:1:1) followed by LiOH (2 mL, 1 M). The mixture was stirred at rt for 5 hours. After acidified with concentrated HCl until pH=4, the slurry was extracted with 30% isopropanol in chloroform, dried with sodium sulfate and concentrated in vacuo to give the enoic acid as a yellow solid. To this enoic acid (100 mg) was added toluene (5 mL) and thionyl chloride (2 mL). The mixture was heated to reflux for 1 hour and the solvent was distilled off under reduced pressure. The residue was taken up with toluene (5 mL) and to it was added anthranilic acid methyl ester (74 mg). The resulting mixture was heated to reflux for additional 1 hour. The solvent was removed and the residue was taken up with DMSO (6 mL). Only part of solid dissolved, the remaining solid was filtered and LC-MS showed it was mainly the desired compound, which was taken up with methanol (18 mL). To this mixture was added tosyl hydrazide (500 mg). The mixture was heated at reflux. After one day, an additional 300 mg of tosyl hydrazide was added. After two and a half days, the resulting mixture was concentrated and dissolved in acetone. The solution was directly purified by biotage (5%-25% ethyl acetate in petroleum ether) to give the anthranilide methyl ester as an oily solid. This methyl ester was dissolved in 5 mL of THF:MeOH:water (3:1:1) followed by LiOH (3 mL, 1 M). The mixture was stirred at rt for 4 hours. After Gilson purification, the acid was obtained as a white solid. To this methyl ether derivative was added 5 mL of dichloromethane and 0.23 mL of borontribromide (0.23 mL, 1N in dichloromethane) at 0° C. After stirring at RT for 2 h, the reaction was quenched by water at 0° C. The mixture was concentrated in vacuo and then dissolved by DMSO. The DMSO solution was purified by Gilson to give Example 47 as a white solid. 1H NMR (acetone-d₆, 500 MHz) δ 11.42 (s, 1H), 8.56 (d, 1H), 8.07 (d, 1H), 7.77 (d, 1H), 7.70 (s, 1H), 7.56 (t, 1H), 7.15 (t, 1H), 6.95 (d, 1H), 6.84 (dd, 1H), 3.34 (t, 2H), 2.88 (t, 2H); LCMS m/z 401 (M−1), 403 (M⁺+1).

### Example 48

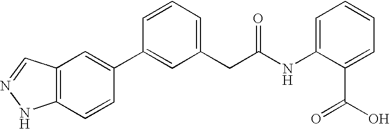

To a solution of 5-aminoindazole (2.03 g, 15.2 mmol) in a mix solution of DMSO (50 mL) and 30% H₂SO₄ (50 mL) at 0° C., was added a solution of sodium nitrate (1.57 g, 22.8 mmol) in 10 mL water dropwisely over 5 mins. Stirred at 0° C. for 1 h, the solution of sodium iodide (7.8 g, 6.8 mmol) in water (5 mL) was added dropwisely. The mixture was stirred for additional 1 h before it was neutralized to pH 6 using 50% NaOH. The compound was extracted with EtOAc and purified on silca gel column chromatography using 20% EtOAc/hexane to obtain the iodide as an off white solid. The mixture of this iodide (100 mg, 0.41 mmol), phenylacetic-3-boronic acid pinacol ester (129 mg, 0.49 mmol), sodium bicarbonate (2 mL, 1N), Pd(PPh₃)₄ (catalytic) in 3 mL dioxane was heated in microwave at 150° C. for 30 mins. After filtration, the filtrate was purified on preparative RPHPLC (Gilson) to obtain the desired acid. A solution of this acid intermediate (13 mg, 0.0515 mmol) in 10 mL anhydrous toluene was treated with 1 mL thionyl chloride, and heated at 100° C. for 1 h. The solvent was removed by distillation and the residue was treated with anthranilic acid in 10 mmL toluene, the resulting mixture was heated to reflux overnight. The solvent was evaporated on rotary evaporator and residue was purified on preparative RPHPLC (Gilson) to obtain Example 48. ¹H NMR (CD₃OD, 600 MHz) δ 8.57 (1H, d), 8.08(1H, s), 8.04(1H, m), 8.01(1H, s), 7.72(1H, m), 7.68(1H, s), 7.58(2H, t), 7.57(1H, t), 7,44(1H, t), 7.33(1H, d), 7.13(1H, t), 3.84(2H, s); LCMS m/z 372.36 (M⁺+1), 370.43 (M⁺−1).

### Example 49

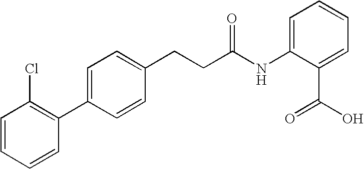

Following the same Suzuki coupling procedures as above, except that the commercially available 2-chlorophenyl boronic acid was used, the desired product was obtained by RP HPLC (Gilson). ¹H NMR (acetone-d₆, 500 MHz): δ 11.5(1H, s), 8.76(1H, d), 8.11(1H, d), 7.59(1H, m), 7.51(1H, d), 7.39(7H, m), 7.13(1H, t), 3.11(2H, t), 2.82(2H, t); LCMS m/z 378 (M−1), 380 (M⁺+1).

### Example 50

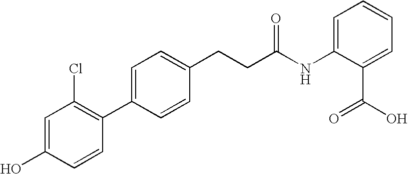

Following the Suzuki procedures. above except that 2-chloro-4-methoxyphenyl boronic acid was used, the biphenyl methyl ether product was prepared. At 0° C., to the biphenyl methyl ether was added dichloromethane (20 mL) and boron tribromide (3 mL, 1 M in dichloromethane). The mixture was then warmed to rt and stirred for 1 h. To this mixture was carefully added water (5 mL) at 0° C. The resulting mixture was concentrated in vacuo and taken up with DMSO. The resulting DMSO solution was purified by RP-HPLC to give Example 50 as a white solid. ¹H NMR (d6-Acetone, 500 MHz) δ 11.3(1H, s), 8.77(1H, d), 8.10(1H, d), 7.59(1H, m), 7.37(2H, d), 7.32(2H, d), 7.21(1H, d), 7.18(1H, d), 7.15(1H, t), 7.00(1H, d), 3.10(2H, t), 2.82(2H, t); LCMS m/z 394 (M−1), 396 (M⁺+1).

### Example 51

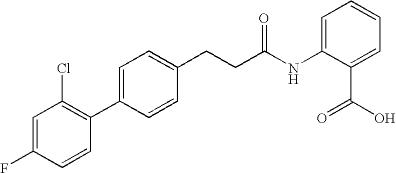

Example 51 was prepared under similar Suzuki conditions described in the examples above. The crude was purified on preparative RPHPLC (Gilson) to obtain the desired product. ¹H NMR (DMSO-d₆, 500 MHz) δ 11.13 (1H, s), 8.49 (1H, d), 7.96(1H, m), 7.59(1H, m), 7.53(1H, m), 7.42(1H, m), 7.34(5H, m), 7.14(1H, t)2.99 (2H, t), 2.78(2H, t); LCMS in/z 398.29(M⁺+1), 396.37(M⁺−1).

### Example 52

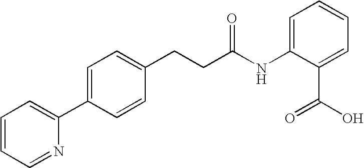

Example 52 was prepared under similar conditions described in the examples above except that DME was used as solvent and potassium hydroxide as base in the Suzuki coupling. The crude was purified on preparative RPHPLC (Gilson) to obtain the desired product as TFA salt. ¹H NMR (acetone-d₆, 500 MHz) δ 11.23(1H, s), 8.75(2H, m), 8.10 (1H, m), 8.05(4H, m), 7.61(1H, t), 7.48(3H, m), 7.16(1H, t), 3.14 (2H, t), 2.83(2H, t). LCMS m/z 347.36 (M⁺+1), 345.42 (M⁺−1).

### Example 53

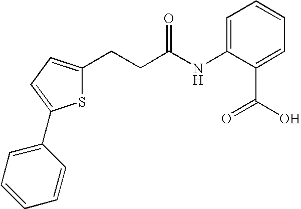

A sealed tube was charged with phenylboronic acid (0.695 g, 5.7 mmol), 2-bromo-thiophene-5-carboxylic acid (1 g, 4.8 mmol), Pd(PPh₃)₄ (277 mg, 0.05 quiv)), sodium carnonate (1.53 g, 3 quiv.) in 20 mL dioxane was heated at 100° C. overnight. The mixture was partitioned between EtOAc and 1N NaOH, the aqueous phase was washed with EtOAc, then acidified to pH 3. The precipitate was collected by filtration and dried to obtain the acid. A solution of this acid intermediate (0.886 g, 4.3 mmol) in 40 mL THF was treated with LiAlH₄ (0.326 g, 8.6 mmol) at 0° C. and stirred for 1.5 h. The reaction was quenched by saturated solution of potassium tartrate. The mixture was extracted with EtOAc, and organic phase was washed with brine and dried over Na₂SO₄. Evaporation of the solvent gave the alcohol. To the solution of this alcohol (0.446 g, 2.3 mmol) in CH₂Cl₂ (20 mL0, at 0° C., was added pyridiniumchlorochromate (0.99 g, 4.6 mmol) in one portion. The mixture was stirred at 23° C. overnight. After evaporation of the solvent, the residue was purified on silica gel chromatography using 5% EtOAc/Hexane to obtain the aldehyde. To a solution of trimethylphosphonoacetate (0.297 mL, 1.8 mmol) in 15 mL THF, at 0° C., was added n-butyl]ithium (1,28mL, 1.6M in hexane, 2.04 mmol) dropwisely. After stirred at 0° C. for 0.5 h, a solution of the above aldehyde intermediate (0.326 g, 1.7 mmol) in TBF (20 mL) was added to the above solution dropwise, and the resulting solution was stirred for 2 h at r.t. After evaporation of the solvent, the residue was purified on silica gel chromatography using 5% EtOAc/hexane to obtain the enoate. A solution of this enoate intermediate (80 mg, 0.327 mmol) and p-toluenesulfonylhydrazide (0.61 g, 3.27 mmol) in methanol (60 mL) was refluxed for 3 days. The compound was purified on silica gel chromatography using 4% EtOAc as eluting solvent to obtain the methyl ester. Following methods described in the above examples, this intermediate was elaborated into Example 53; ¹H NMR (DMSO-d₆, 500 MHz) δ 11.14(1H,s), 8.47(1H,d), 7.97(1H, d), 7.59(3H, m), 7.38(2H, t), 7.30 (1H, d), 7.25(1H, t), 7.15(1H, t), 6.90(1H, d), 3.16(2H, t), 2.80(2H, t); LCMS m/z 352.31 (M⁺+1), 350.40 (M⁺−1).

### Example 54

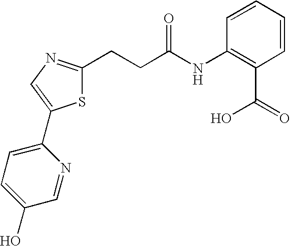

A mixture of 2-thiazolecarboxaldehyde (1.1 g), ethyleneglycol (1.5 g), p-toluenesulfonic acid (0.18 g) and toluene (50 mL) was heated at reflux with a Dean-Stark trap. After 1 h, to the cooled mixture were added ethyl acetate (100 mL) and saturated sodium bicarbonate (50 mL) and water (15 mL). The aqueous layer was extracted with ethyl acetate (100 mL×2). The combined organic layers were dried with sodium sulfate and concentrated in vacuo. The residue was purified by Biotage (5-20% ethyl acetate in hexanes) to give the acetal as a yellow oil. To a solution of this acetal intermediate (1.1 g) in 50 mL of THF was added n-BuLi (5.3 mL, 1.6 M in hexane) at −78° C. After 45 min, to this solution was added tributyltin chloride (2.7 g, 2.3 mL). The mixture was warmed to 0° C. over 30 min and quenched with water. The mixture was extracted with ethyl acetate. The organic layer was combined, dried with sodium sulfate and concentrated in vacuo to give a brown oil, which was further purified by Biotage (5-10% ethyl acetate in hexane) to give the stannane as a brown oil. A mixture of this stannane intermediate (380 mg), 2-bromo-5-nitropyridine (190 mg) and toluene (3 mL) was degassed with argon for 3 min. To the mixture were then added Pd(PPh₃)₄ and CuI (8 mg). The resulting mixture was heated at 100° C. for 2 days. To this resulting mixture were added ethyl acetate, water and brine. The organic layer was dried with sodium sulfate and concentrated. The residue was purified by Biotage to give the biaryl intermediate as a brown solid. To a mixture of this biaryl intermediate (120 mg) in 10 mL of THF was added HCl (2 mL, 1N). The mixture was heated at reflux for 6 h. The crude mixture was purified by Biotage to provide the aldehyde. To a solution of trimethylphosphonoacetate (0.39 mL) in 50 mmL of THF was added n-butyl]ithium (1.65 mL, 1.6 M in hexane) at 0° C. After 15 min, the mixture was warmed to 23° C., and to this solution was added a solution of the biaryl aldehyde (500 mg) in 1 mL of THF. The resulting slurry was stirred at 23° C. for 2 h, and to this mixture was added ethyl acetate and water. The organic layer was then dried with sodium sulfate and concentrated to give the enoate as a yellow solid. To the methyl enoate (470 mg) were added 50 mL of THF:methanol:water (3:1:1) and 1 N lithium hydroxide solution (10 mL). After 12 h, the clear dark brown solution was concentrated to about 15 mL. The aqueous layer was acidified with concentrated HCl until precipitate appeared. The mixture was filtered, and the filtrate was purified by RPHPLC to give the enoic acid as a bright yellow solid. To this acid (129 mg) was added 2 mL of thionyl chloride. The resulting clear solution was heated at 80° C. for 60 min and thionyl chloride was removed in vacuo. To the residue were added toluene (8 mL) and anthranilic acid (90 mg). The mixture was heated at 110° C. for 1 h. The resulting slurry was filtered. The collected solid was washed with acetone to give the enamide as a yellow solid. To a slurry of this nitro enamide (60 mg) in 10 mL of methanol was added 35 mg of Pd/C (10%). The mixture was stirred under 1 atm of hydrogen gas for 3 h. The slurry was filtered, and the filtrate was washed with acetone and methanol. The filtrate was concentrated to give the aniline as a sticky yellow oil. To this aniline (41 mg) and 2 mL of 1N H₂SO₄ was added sodium nitrite (46 mg) at 0° C. The slurry was warmed to 23° C. and stirred for 15 min. The mixture contained some insoluble red solid. The mixture was then heated at 80° C. for 5 min. The solution became clear and the color faded. The mixture was filtered and the solid was dissolved in DMSO. The aqueous filtrate and DMSO solution were purified by Gilson to give the desired product as an off-white solid. ¹H NMR (acetone-d₆, 500 MHz) δ 11.4 (1H, s), 8.75 (1H, d), 8.17 (1H, d), 8.11 (1H, d), 8.05 (1H, s), 7.72 (1H, d), 7.61 (1H, t), 7.29 (1H, dd), 7.16 (1H, t), 3.42 (2H, t), 3.02 (2H, t); LCMS m/z 370 (M⁺+1).

### Example 55

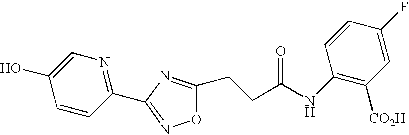

To a mixture of 5-bromo-2-cyanopyridine (1 g, 5.5 mmol), cesium carbonate (3.6 g, 11 mmol), 4-methoxybenzyl alcohol (1.5 g, 10.9 mmol) in a solution of 20 mL of toluene was quickly added 1,10-phenanthroline (98 mg, 0.55 mmol) and copper(I) iodide (52 mg, 0.27 mmol) under nitrogen. The reaction mixture was heated at 120° C. overnight. To the mixture was then added water (150 mL), and partitioned twice with ethyl acetate (2×100 mL). The aqueous layer was then extracted twice with dichloromethane (2×100 mL). The combined organic phases were dried with sodium sulfate and concentrated in vacuo. The residue was dissolved in DMSO and purified by RPHPLC to give 4-(4-methoxybenzyloxy)-2-cyanopyridine as a pale yellow solid. To a slurry of this intermediate (60 mg, 0.25 mmol) and hydroxylamine hydrochloride (38 mg, 0.55 mmol) in 8 mL of ethanol, was added 0.17 mL of 3 N sodium hydroxide aqueous solution. The reaction mixture was stirred at 23° C. overnight. The residue was purified by RPHPLC to give 4-(4-methoxybenzyloxy)-2-hydroxyamidinylpyridine as a white solid. To a solution of this intermediate (180 mg, 0.66 mmol) in 8 mL of pyridine was added the mono acyl chloride (199 mg, 1.32 mmol). The resulting mixture was heated at 130° C. for 30 min. After removing most solvent, the residue was diluted with dichloromethane and purified by Biotage chromatography (10-50% ethyl acetate in hexane) to afford the oxadiazole intermediate as a white solid. To this oxadiazole intermediate (126 mg, 0.34 mmol) was added 4 mL of a mixture of trifluoroacetic acid and dichloromethane (1:1) at 23° C. After 30 min, the purple colored reaction mixture was concentrated in vacuo. The residue was used directly in the next step without further purification. To a mixture of this crude hydroxypyridine methyl ester in 20 mL of THF:methanol:water (3:1:1), was added a solution of lithium hydroxide (5 mL, 1N). After 1 h, most of the volatiles were removed in vacuo. To the residue was added 15 mL of water, and the mixture was extracted with 30% isopropanol in chloroform (3×50 mL). The combined organic phase was concentrated, and the residue was purified by RPHPLC to give the acid intermediate as a colorless oil. To a mixture of this acid (68 mg, 0.29 mmol) in 10 mL of dichloromethane, were added triethylamine (102 mg, 0.14 mL) and tert-butyldimethylsilyl chloride (109 mg, 0.73 mmol) at 23° C. After 3 h the mixture was quenched with water, and the aqueous layer was extracted with dichloromethane. The combined organic phase was concentrated in vacuo to give the bis- TBS-protected product as a brown oil, which was directly used in the next step. In an ice bath, to this intermediate in dichloromethane (5 mL), was added one drop of DMF, and then a solution of oxalyl chloride (0.28 mL, 2 N in dichloromethane). After 1.5 h, the mixture was warmed to 23° C. and stirred for another 1.5 h. The resulting mixture was concentrated in vacuo, and then this acid chloride intermediate was reacted with the commercially available fluoro anthranilic acid derivative. The desired product was obtained following procedures in the Examples above. ¹H NMR (CD₃OD, 500 MHz) δ 11.2 (1H, s), 8.68 (1H, dd), 8.32 (1H, d), 7.95 (1H, d), 7.77 (1H, dd), 7.40 (2H, m), 3.37 (2H, t), 3.05 (2H, t); LCMS m/z 373 (M⁺+1).

### Example 56

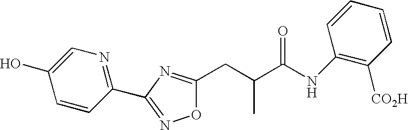

To a solution of ethyl 2-methyl-4-pentenoate (3.1 g) and NMO (6.4 g) in 20 mL of dichloromethane, was added OsO₄ (2.7 mL, 4% in water). After 12 h, to the mixture were added water (100 mL), dichloromethane (200 mL), and 30% isopropanol in chloroform (100 mL). The organic layer was concentrated. To the residue was added acetone and sodium periodate (9.3 g) in 50 mL of water. The white precipitate was formed and the slurry was stirred for 30 min and filtered. The filtrate was concentrated and extracted with dichloromethane (200 mL). The organic layer was dried with sodium sulfate and concentrated. The residue was purified by Biotage to give the aldehyde as a colorless oil. To this oil was added 15 mL of t-butanol, 2-methylbutene (10 mL), and a solution of sodium dihydrophosphate (12 g) and sodium chlorite (9 g, 80%) in 50 mL of water. After 1.5 h, the mixture was basified with NaOH. The organic layer was removed and the aqueous layer was acidified with HCl until pH=3. The mixture was extracted with ethyl acetate. The organic layer was dried with sodium sulfate and concentrated to give the monoacid as a dark oil. To a solution of this monoacid (250 mg) in 5 mL of toluene was added thionyl chloride (1.5 mL). The mixture was heated at 70° C. for 1 h, and the volatiles were removed in vacuo and azetroped with toluene. To the residue was added the intermediate, 4-(4-methoxybenzyloxy)-2-hydroxyamidinylpyridine, from EXAMPLE 55 above (427 mg) and pyridine (3 mL). The resulting mixture was heated at 130° C. for 2 h. The crude was purified by Biotage (5-50% ethyl acetate in hexane) to give a mixture of ring-cyclized and ring-opened product. The resulting mixture was heated at reflux in ethanol (20 mL) for 2 days. After removing solvent, the fully cyclized oxadiazole product was obtained as a light yellow oil. To this ethyl ester (155 mg) were added 10 mL of THF:methanol:water (3:1:1) and 1N lithium hydroxide solution (4 mL). After 2 h, the mixture was concentrated. To the aqueous residue was added HCl until pH=4. This mixture was extracted with 30% isopropanol in chloroform (20 mL). The combined organic layers were dried with sodium sulfate and concentrated in vacuo to give the acid as a brown oil. At 0° C., to a solution of this acid intermediate (30 mg) in 2 mL of dichloromethane was added 1 drop of DMF and oxalyl chloride (0.1 mL, 2 M in dichloromethane). The resulting solution was stirred for 30 min. After removing the volatiles, the residue was dissolved in 2 mL of dichloromethane. To this solution was added methyl anthranilide (24 mg). The resulting mixture was stirred overnight. To this mixture was added TFA (1 mL). After 30 min, the mixture was purified by Gilson to give a colorless oil. To a solution of this methyl ester (19 mg) in 2 mL of THF:methanol:water (3:1:1) was added 1.2 mmL of LiOH (1N). After 5 h, the mixture was acidified with concentrated HCl to pH=3. The mixture was extracted with 30% isopropanol in chloroform. The organic layer was concentrated, and the residue was purified by Gilson to give the desired product as a white solid. ¹H NMR (acetone-d₆, 500 MHz) δ 11.5 (1H, s), 8.68 (1H, d), 8.32 (1H, m), 8.11 (1H, d), 7.95 (1H, m), 7.59 (1H, t), 7.37 (1H, m), 7.16 (1H, t), 3.46 (1H, dd), 3.26 (1H, m), 3.15 (1H, dd), 1.46 (3H, d); LCMS m/z 369 (M⁺+1).

### Example 57

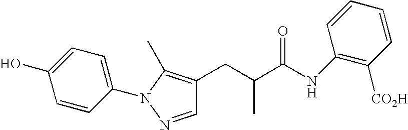

A solution of the commercially available aldehyde intermediate shown in Scheme 14 (1.45 g, 6.7 mmol) and ethyl triphenylphosphonium methyl acetate (3.1 g, 8.1 mmol) in 15 mL of toluene was heated at 130° C. for 16 h. The mixture was directly purified by Biotage (5-20% ethyl acetate in hexane) to give the enoate as a light yellow solid. This intermediate (1.74 g, 5.8 mmol) and Pd/C (10%, 170 mg) in 200 mL of methanol was stirred under 1 atm of hydrogen gas (balloon) for 12 hrs. The slurry was filtered and concentrated in vacuo. The residue was dissolved in ethanol/methanol (1:1) and purified by chiral OJ-H (9 mL/min, 28% isopropanol/heptane, isocratic, 40 min/run) to give the enantiomers as white solids. Eluting times were 18 min and 22 min using analytical Chiralcel-OJ, 25% isopropanol in heptane (isocratic). The ethyl ester (400 mg, 1.32 mmoL) was combined with concentrated HCl (2 mL) and 4 mL of acetic acid, and was heated at 80° C. for 3 h. The mixture was concentrated in vacuo, and to it was added 15 mL of water. The mixture was extracted with 30% isopropanol/chloroform (50 mL×4). The organic layer was dried with sodium sulfate and concentrated in vacuo to give the acid product as a white solid. To this acid (295 mg) was then added thionyl chloride (2 mL) and toluene (5 mL). The mixture was heated at 80° C. for 1.5 h, and the volatiles were removed in vacuo, and azetroped with toluene. To the residue was added anthranilic acid (369 mg). The resulting mixture was heated at 80° C. for 1.5 h. The mixture was concentrated, and to the residue was added ethyl acetate (300 mL). The mixture was washed with 4N HCl (100 mL×3). The organic layer was dried with sodium sulfate and concentrated to give the methyl ether as a white solid. At 0° C., to this intermediate (297 mg) was added 25 mL of dichloromethane and 7 mL of BBr₃ (7 mL, 1 N in dichloromethane). The mixture was slowly warmed to 23° C. and stirred for 1.5 h. The mixture was re-cooled to 0° C. and quenched with water (2 mL). The mixture was then warmed to 23° C. and concentrated in vacuo. The residue was diluted with DMSO and methanol (1:5) and then purified by Gilson to give the desired product as a light pink solid. ¹H NMR (CD₃OD, 500 MHz) δ 11.4 (1H, s), 8.57 (1H, d), 8.06 (1H, dd), 7.54 (1H, t), 7.44 (1H, s), 7.13 (1H, t), 7.10 (2H, d), 6.85 (2H, d), 3.33 (1H, m), 2.83 (1H, m), 2.73 (2H, m), 2.14 (3H, s), 1.32 (3H, d); LCMS m/z 380 (M⁺+1).

### Example 58

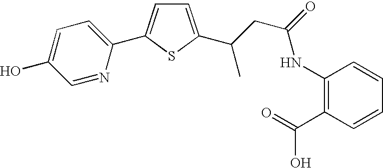

A mixture of the commercially available ketone (1.64 g), methyl triphenylphosphoranylidene acetate (2.8 g), and 20 mL of toluene was heated at 150° C. for 2 days. The mixture was purified by Biotage (5% ethyl acetate in hexane) to afford the enoate (cis:trans=1:1) as a white solid. The hydrolysis of this enoate, and the subsequent amide formation, followed the procedures described in the Examples above to provide a yellow oil. A solution of the bromide (1.24 g), hexamethyl ditin (1.6 g) in 10 mL of THF was degassed with argon, and to this solution was added Pd(PPh₃)₄ (151 mg). The mixture was heated at 80° C. overnight. The resulting stannane mixture was used directly for the subsequent Stille coupling, following procedures described in the above Examples. Following similar procedures as described in EXAMPLE 54, after hydrogenation, conversion of the amino group to the hydroxyl group, and hydrolysis, the desired product was obtained as a brown oil. ¹H NMR (acetone-d₆, 500 MHz) δ 11.3 (1H, s), 8.75 (1H, d), 8.13 (1H, d), 8.10 (1H, d), 7.63 (1H, d), 7.60 (1H, t), 7.33 (1H, d), 7.25 (1H, dd), 7.16 (1H, t), 6.91 (1H, d), 3.68 (1H, m), 2.83 (1H, dd), 2.75 (1H, dd), 1.45 (3H, d); LCMS m/z 383 (M⁺+1).

### Example 59

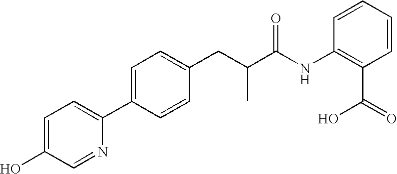

A mixture of 4-methylphenyl boronic acid (680 mg), 2-bromo-5-nitropyridine (1.02 g), Pd(PPh₃)₄ (50 mg), NaHCO₃ (7.5 mL, 1M in water), and dioxane (7.5 mL) was heated at 100° C. overnight. After being diluted with ethyl acetate (100 mL) and dichloromethane (10 mL), the mixture was washed with water. The organic layer was dried with sodium sulfate and concentrated. The residue was purified by Biotage eluting with 5% dichloromethane and 5% ethyl acetate in hexane to give the biaryl intermediate as a white solid. To a mixture of this intermediate (0.90 g) in 2:1 of CCl₄ and 1,2-dichloroethane, was added NBS (1.2 g). The mixture was subjected to light to initiate radical formation. Without external heating, refluxing of the solvent was observed. After 30 min, the mixture was washed with saturated NaHCO₃ solution and water. The organic layer was dried with sodium sulfate and concentrated to give the monobromide as a pale yellow solid containing a small amount of bis-bromo byproduct. To sodium hydride (66 mg, 60%) in 5 mL of THF was added diethyl methyl malonate (261 mg) at 0° C. After 15 min, to the resulting solution was added the bromide intermediate (300 mg). After 6 h, to the mixture were added 15 mL of water and 20 mL of ethyl acetate. The aqueous layer was extracted thrice with ethyl acetate (15 mL). The organic fractions were combined and dried with sodium sulfate. After the removal of solvent, the yellow oil residue was purified by Biotage (2-20% ethyl acetate in hexane) to give the diester as a yellow oil. To this intermediate (0.92 g) were added 40 mL of THF:methanol:water (3:1:1) and 1N lithium hydroxide solution (15 mL). After 8 h at 80° C., the mixture was concentrated. To the aqueous residue was added HCl until pH=4. This mixture was extracted with 30% isopropanol in chloroform. The combined organic layers were dried with sodium sulfate and concentrated in vacuo to give the diacid as a yellow solid. A solution of the diacid (0.8 g) in 12 mL of DMF was heated at 170° C. in a MicroWave for 2 min. The solution was purified by RPHPLC to give the nitroacid as a yellow solid. The same reaction conditions as described for the preparation of EXAMPLE 54 provided the desired product as a yellow oily solid. ¹H NMR (CD₃OD, 500 MHz) δ 8.52 (1H, d), 8.19 (1H, d), 8.04 (2H, m), 7.89 (1H, dd), 7.72 (2H, d), 7.53 (1H, m), 7.47 (2H, d), 7.12 (1H, m), 3.11 (1H, dd), 2.93 (1H, dd), 2.85 (1H, m), 1.33 (3H, d); LCMS m/z 377 (M⁺+1).

### Example 60

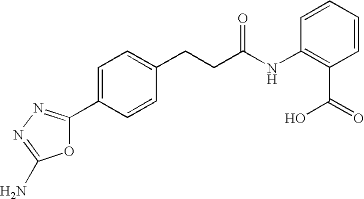

Hydrazine (51% in water, 6.4 mL, 5 eq, 104 mmol) was added to a methanol (140 mL) solution of methyl-4- iodobenzoate (5.48 g, 1 eq, 20.92 mmol) and stirred for 4 h. The hydrazide product resulted as a white precipitate, and was filtered after cooling the solution to 0° C. Sodium bicarbonate (0.353 g in 4.2 mL water, 1 eq) was added to a dioxane (14 mL) solution of this intermediate (1.1 g, 4.2 mmol) in 5 min, followed by adding cyanogen bromide (0.56 g 5.25 mmol, 1.25 eq). The solution was stirred for 15 h. The amino oxadiazole product resulted as a white precipitate, and was obtained by filtration. This intermediate (200 mg, 0.7 mmol, 1 eq), along with the acrylamide of methyl anthranilate (230 mg, 1.15 mmol, 1.6 eq), Pd(OAc)₂ (8 mg, 0.05 eq), and P(O-tol)₃ (22 mg, 0.1 eq) in Et₃N (0.3 mL, 3 eq) and DMF (0.4 mL) was heated to 100° C. for 4 h. After the reaction solution was cooled to 23° C., LiOH (3 mL, 0.5M. 2eq) was added and stirred for another 2 h. The solution was filtered, and the residue was purified by RPHPLC to obtain the enamide product. Hydrogen gas (balloon) was charged with this intermediate (10 mg) and Pd/C (1 mg) in methanol (8 mL) for 4 h to obtain the desired product after filtration. ¹H NMR (CDCl₃, 500 MHz) δ 11.25 (s, 1H), 8.52 (d, 1H), 7.98 (d, 1H), 7.72 (d, 2H), 7.45 (t, 1H), 7.29 (d, 2H), 7.00 (t, 1H), 3.04 (t, 2H), 2.69 (t, 2H); LCMS m/z 353 (M⁺+1).

### Example 61

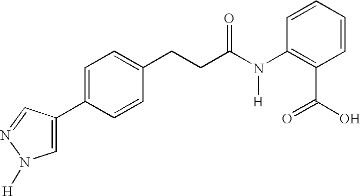

To the commercially available [4-(2-methoxycarbonylethyl)-phenyl]boronic acid (0.5 g, 2.4 mmol) in 5 mL of dioxane, was added (N-benzyl)-4-iodopyrazole (1.36 g, 4.8 mmol) followed by triethylamine (729 mg, 7.2 mmol), and tetrakis-triphenylphosphine palladium (256 mg, 0.24 mmol). The resulting mixture was heated in the MicroWave for 10 minutes at 100° C. Following the reaction completion, the mixture was concentrated in vacuo, and purified by flash chromatography (Biotage 40M) to give the desired product. To a solution of the ester (720 mg, 2.24 minol) in 5 mL of THF//H₂O (2:1), was added sodium hydroxide (448 mg, 11.2 mmol). The biphasic solution was allowed to stir for 12 h. Upon desired completion, the reaction was concentrated in vacuo, diluted with 10 mL of water, cooled to 0° C. and acidified with concentrated HCl to a pH of 3. The acidic solution was extracted three times with ethyl acetate (10 mL) and the organic extracts were dried with sodium sulfate and concentrated in vacuo. Without further purification, the carboxylic acid (90 mg, 0.19 mmol) was treated with 5ml of toluene/SOCl₂ (5:1) and heated to 90° C. for 2 h. Upon completion, the reaction mixture was concentrated, diluted with CH₂Cl₂ and ethyl anthranilate (1.48 g, 8.9 mmol) was added dropwise and the reaction mixture was allowed to stir for 2 h at room temperature. Following the reaction completion, the reaction mixture was concentrated and purified via flash chromatography (Biotage 40 M). To a solution of the ester (45 mg, 0.10 mmol) in 5 mL of THF//H₂O (2:1), was added sodium hydroxide (48 mg, 1.2 mmol). The biphasic solution was allowed to stir for 12 h. Upon desired completion, the reaction was concentrated in vacuo, diluted with 3 mL of water, cooled to 0° C. and acidified with concentrated HCl to a pH of 3. The acidic solution was extracted three times with ethyl acetate (5 mL) and the organic extracts were dried with sodium sulfate and concentrated in vacuo. Without further purification, to the anthranilic acid derivative (30 mg, 0.071 mmol) in dimethylsulfoxide (1 mL) was bubbled pure oxygen for 5 minutes. With a positive flow of oxygen, potassium tert-butoxide in tetrahydrofuran(1M, 0.71 mmol) was added dropwise to the reaction at room temperature. The reaction was allowed to stir for 1 h at room temperature with a continuous flow of oxygen through the solution. Upon completion, anhydrous hydrochloric acid in dioxane (lmil) was added dropwise to the reaction mixture, and the mixture was allowed to stir for 20 minutes. The reaction mixture was filtered and purified by preparative RPHPLC on a Gilson system to afford the desired product. ¹H NMR (DMSO-d₆, 500 MHz) δ 11.13 (s, 1H), 8.48 (d, 1H), 7.97 (d, 1H), 7.69 (m, 2H), 7.58 (m, 1H), 7.31 (d, 2H), 7.14( t, 1H), 6.66 (s, 1H), 2.97 (m, 2H), 5.49 (m, 2H), ; LCMS m/z 336 (M⁺+1).

### Example 62

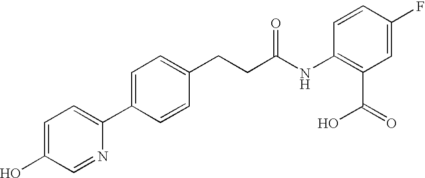

Following a similar procedure as described above for EXAMPLE 6, the desired product was obtained. ¹H NMR (CD₃OD, 500 MHz) δ 8.56 (1H, dd), 8.20 (1H, d), 8.08 (1H, d), 7.92 (1H, dd), 7.75 (3H, m), 7.52 (2H, d), 7.33 (2H, m), 3.15 (2H, t), 2.82 (2H, t); LCMS m/z 381 (M⁺+1).

### Example 63

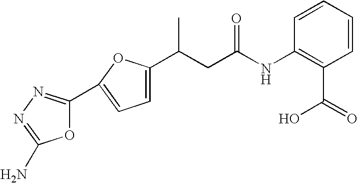

Following a similar procedure as described above for EXAMPLE 60, the commercially available bromofuran methyl ester shown in Scheme 18, was transformed into the desired product. ¹H NMR (CD₃OD, 500 MHz) δ 8.51 (d, 1H), 8.05 (d, 1H), 7.53 (t, 1H), 7.13 (t, 1H), 6.89 (d, 1H), 6.34 9d, 1H), 3.52 (m, 1H), 2.88 (m, 1H), 2.66 (m, H), 1.40 (d, 3H); LCMS m/z 355 (M⁺−1).

Moreover, the nicotinic acid receptor has been identified and characterized in WO02/084298A2 published on Oct. 24, 2002 and in Soga, T. et al., Tunaru, S. et al. and Wise, A. et al. (citations above).

Numerous DP receptor antagonist compounds have been published and are useful and included in the methods of the present invention. For example, DP receptor antagonists can be obtained in accordance with WO01/79169 published on Oct. 25, 2001, EP 1305286 published on May 2, 2003, WO02/094830 published on Nov. 28, 2002 and WO03/062200 published on Jul. 31, 2003. Compound AB can be synthesized in accordance with the description set forth in WO01/66520A1 published on Sep. 13, 2001; Compound AC can be synthesized in accordance with the description set forth in WO03/022814A1 published on Mar. 20, 2003, and Compounds AD and AE can be synthesized in accordance with the description set forth in WO03/078409 published on Sep. 25, 2003. Other representative DP antagonist compounds used in the present invention can be synthesized in accordance with the examples provided below.

### DP Example 1

### [5-[(4-Chlorophenyl)thio]-4-(methylsulfonyl)-6,7,8,9-tetrahydropyrido[3,2-b]indolizin-6-yl]acetic acid (Compound G)

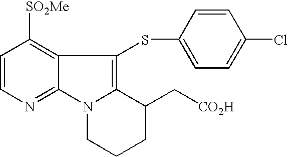

### Step 1 4-Chloronicotinaldehyde

The title compound was prepared as described by F. Marsais et al., J. Heterocyclic Chem., 25, 81 (1988).

### Step 2 4-(Methylthio)nicotinaldehyde

To a solution of NaSMe (9.5 g, 135 mmol) in MeOH (250 mL) was added the 4-chloronicotinaldehyde (13.5 g, 94.4 mmol) of Step 1 in MeOH (250 mL). The reaction mixture was maintained at 60° C. for 15 min. The reaction mixture was poured over NH₄Cl and EtOAc. The organic phase was separated, washed with H₂O and dried over Na₂SO₄. The compound was then purified over silica gel with 50% EtOAc in Hexanes to provide the title compound.

### Step 3 Methyl (2Z)-2-azido-3-[4-(methylthio)pyridin-3-yl]prop-2-enoate

A solution of 4-(methylthio)nicotinealdehyde (4.8 g, 31 mmol) and methyl azidoacetate (9.0 g, 78 mmol) in MeOH (50 mL) was added to a solution of 25% NaOMe in MeOH (16.9 mL, 78 mmol) at −12° C. The internal temperature was monitored and maintained at −10° C. to −12° C. during the 30 min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum to give the title compound as a beige solid, which contained some salts. The compound is then purified over silica gel with EtOAc.

### Step 4 Methyl 4-(methylthio)-1H-pyrrolo[2,3-b]pyridine-2-carboxylate

A suspension of the compound of Step 3 (0.40 g, 1.6 mmol) in xylenes (16 mL) was heated slowly to 140° C. After a period of 15 min. at 140° C., the yellow solution was cooled to room temperature. Precaution must be taken due to the possibility of an exotherme due to the formation of nitrogen. The suspension was then cooled to 0° C., filtered and washed with xylene to provide the title compound.

### Step 5 Ethel 4-(methylthio)-6-oxo-6,7,8,9-tetrahydropyrido[3,2-b]indolizine-7-carboxylate

To a solution of the compound of Step 4 (0.35 g, 1.6 mmol) in DMF (20 mL) at 0° C. was added NaH (1.2 eq.). After a period of 5 min., nBu₄NI (0.10 g) and ethyl 4-bromobutyrate (0.40 mL). were added. After a period of 1 h at room temperature, the reaction mixture was poured over saturated NH₄Cl and EtOAc. The organic phase was separated, washed with H₂O and dried over NaSO₄. After evaporation the crude product was purified by flash chromatography. The bis ester was then dissolved in THF (7.0 mL) and a 1.06 M of THF solution of potassium tert-butoxide (2.2 mL) was added at 0° C. After a period of 1 h at room temperature, the reaction mixture was then poured over saturated NH₄Cl and EtOAc. The organic phase was separated, dried over Na₂SO₄ and evaporated under reduced pressure to provide the title compound as a mixture of ethyl and methyl ester.

### Step 6 4-(Methylthio)-8,9-dihydropyrido[3,2-b]indolizin-6(7H)-one

To the compound of Step 5, (0.32 g) were added EtOH (8.0 mL) and concentrated HCl (2.0 mL). The resulting suspension was refluxed for 5 h. The reaction mixture was partitioned between EtOAc and Na₂CO₃. The organic phase was separated and evaporated to provide the title compound.

### Step 7 Ethyl (2E, 2Z)-[4-(methylthio)-8,9-dihydropyrido[3,2-b]indolizin-6(7H)-ylidene]ethanoate

To a DMF solution (12 mL) of triethyl phosphonoacetate (0.45 g, 2.17 mmol) were added 80% NaH (0.06 g, 2.00 mmol) and the compound of Step 6 (0.22 g, 1.00 mmole). After a period of 4 h at 55° C., the reaction mixture was poured over saturated NH₄Cl and EtOAc. The organic phase was separated and evaporated under reduced pressure. The crude product was purified by flash chromatography to afford the title compound.

### Step 8 Ethyl [4-(methylthio)-6,7,8,9-tetrahydropyrido[3,2-b]indolizin-6-yl]acetate

The compound of Step 7 was dissolved in MeOH-THF using heat for dissolution. To the previous cooled solution was added at room temperature PtO₂ and the resulting mixture was maintained for 18 h under an atmospheric pressure of hydrogen. The reaction mixture was filtered carefully over Celite using CH₂Cl₂. The filtrate was evaporated under reduced pressure to provide the title compound. Alternatively, the compound of Step 7 can be hydrogenated with Pd (OH)₂ in EtOAc at 40 PSI of H₂ for 18 h.

### Step 9 Ethyl [4-(methylsulfonyl)-6,7,8,9-tetrahydropyrido[3,2-b]indolizin-6-yl]acetate

To the compound of Step 8 (0.08 g, 0.27 mmol) in MeOH (3.0 mL) were added Na₂WO₄ (0.10 g) and 30% H₂O₂ (600 μL). After a period of 1 h, the reaction mixture was partitioned between H₂O and EtOAc. The organic phase was washed with H₂O, separated and evaporated. The title compound was purified by flash chromatography.

### Step 10 Ethyl [5-[(4-chlorophenyl)thio]-4-(methylsulfonyl)-6,7,8,9-tetrhydropyrido[3,2-b]indolizin-6-yl]acetate

To a 1,2-dichloroethane solution (2.0 mL) of 4,4′-dichlorodiphenyl disulfide (0.24 g) was added SO₂Cl₂ (50 μL). To the compound of Step 9 (0.05 g) in DMF (2.0 mL) was added the previous mixture (≈180 μL). The reaction was followed by ¹H NMR and maintained at room temperature until no starting material remained. The reaction mixture was poured over saturated NaHCO₃ and EtOAc. The organic phase was separated, evaporated and the title compound purified by flash chromatography.

### Step 11 [5-[(4-Chlorophenyl)thio]-4-(methylsulfonyl)-6,7,8,9-tetrahydropyrido [3,2-b]indolizin-6-yl]acetic acid

To the compound of Step 10 dissolved in a 1/1 mixture of THF-MeOH was added 1N NaOH. After a period of 18 h at room temperature, the reaction mixture was partitioned between saturated NH₄Cl and EtOAc. The organic phase was separated, dried over Na₂SO₄ and evaporated to provide the title compound.

¹H NMR (500 MHz, acetone-d₆) δ 11.00 (bs, 1H), 8.60 (d, 1H), 7.80 (d, 1H), 7.20 (d, 2H), 7.00 (d, 2H), 4.65 (m, 1H), 4.20 (m, 1H), 3.75 (m, 1H), 3.35 (s, 3H), 2.80 to 2.10 (m, 6H).

### DP Example 2

### [5-[(4-Chlorophenyl)thio]-4-(methylthio)-6,7,8,9-tetrahydropyrido[3,2-b]indolizin-6-yl]acetic acid (Compound H)

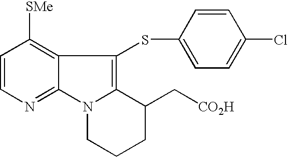

The title compound can be prepared from the compound of Example 1, Step 8 in a similar manner as described in Example 1, Step 10 and 11. m/z 418.

### DP Example 3

### [5-[(3,4-Dichlorophenyl)thio]-4-(methylsulfonyl)-6,7,8,9-tetrahydropyrido[3,2-b]indolizin-6-yl]acetic acid (Compound I)

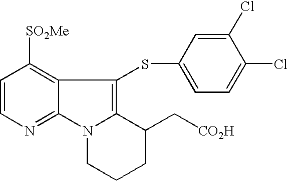

The title compound was prepared as described in Example 1 using bis(3,4-dichlorophenyl)disulfide in Step 10.

¹H NMR (500 MHz, acetone-d₆) δ 8.55 (d, 1H), 7.85 (d, 1H), 7.35 (d, 1H), 7.15 (s, 1H), 6.95 (d, 1H), 4.60 (m, 1H), 4.15 (m, 1H), 3.80 (m, 1H), 3.40 (s, 3H), 2.80 to 2.10 (m, 6H). m/z 484.

The enantiomers were separated on a Chiralecel OD column 25 cm×20 mm using 30% isopropanol 17% ethanol 0.2% acetic acid in hexane, flow rate 8 ml/min. Their pureties were verified on a Chiralecel OD column 25 cm×4.6 mm using 35% isopropanol 0.2% acetic acid in hexane, flow rate 1.0 ml/min. More mobile enantiomer Tr=9.7 min, less mobile enantiomer Tr 11.1 min.

### DP Example 4

### [5-(4-Chlorobenzoyl)-4-(methylsulfonyl)-6,7,8,9-tetrahydropyrido[3,2-b]indolizin-6-yl]acetic acid (Compound J)

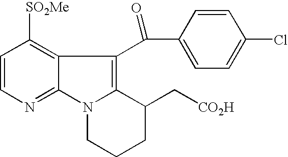

### Step 1 Ethyl [5-(4-chlorobenzoyl)-4-(methylthio)-6,7,8,9-tetrahydropyrido[3,2-b]indolizin-6-yl]acetate

To a solution of 4-chlorobenzoyl chloride (0.30 g, 1.7 mmol) in 1,2-dichloethane (6.0 mL) was added AlCl₃ (0.24 g, 1.8 mmole). After a period of 5 min. a solution of ethyl [4-(methylthio)-6,7,8,9-tetrahydropyrido[3,2-b]indolizin-6-yl]acetate from Example 1 Step 8 (0.15 g, 0.47 m 1,2-dichloroethane (6.0 mL) was added to the previous mixture. After a period of 4 h, at 80° C., the reaction mixture was partitioned between EtOAc and NaHCO₃. The organic phase was separated, dried over Na₂SO₄ and evaporated. The title compound was purified by flash chromatography.

### Step 2 Ethyl [5-(4-chlorobenzoyl)-4-(methylsulfonyl)-6,7,8,9-tetrahydropyrido[3,2-b]indolizin-6-yl]acetate

To a solution of ethyl[5-(4-chlorobenzoyl)-4-(methylthio)-6,7,8,9-tetrahydropyrido[3,2-b]indolizin-6yl]acetate (0.12 g, 0.27 mmole) in MeOH (5.0 mL) were added Na₂WO₄ (0.1 g) and 30% H₂O₂ (300 μL). The reaction mixture was stirred at 55° C. for 1 h. The reaction mixture was then partitioned between H₂O and EtOAc. The organic phase was washed with H₂O, dried over Na₂SO₄ and evaporated. The title compound was purified by flash chromatography.

### Step 3 [5-(4-Chlorobenzoyl)-4-(methylsulfonyl)-6,7,8,9-tetrahydropyrido[3,2-b]indolizin-6-yl]acetic acid

Ethyl [5-(4-chlorobenzoyl)-4-(methylsulfonyl)-6,7,8,9-tetrahydropyrido[3,2-b]indolizin-6yl]acetate was treated as described in Example 1 Step 11 to provide the title compound.

¹H NMR (500 MHz, acetone-d₆) δ 8.55 (d, 1H), 7.90 (d, 2H), 7.65 (d, 1H), 7.45 (d, 2H), 4.55 (m, 1H), 4.25 (m, 1H), 3.45 (m, 1H), 3.20 (s, 3H), 2.05 to 3.00 (m, 6H). m/z 446.

### DP Example 5

### [5-(4-Bromophenyl)thio]-4-(methylsulfonyl)-6,7,8,9-tetrahydropyrido[3,2-b]indolizin-6-yl]acetic acid (Compound K)

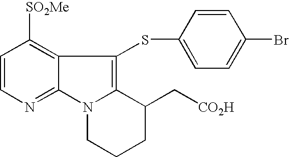

The title compound was prepared as described in Example 1 using 4,4′-dibromodiphenyl disulfide.

¹H NMR (500 MHz, Acetone-d₆) δ 8.60 (d, 1H), 7.80 (d, 1H), 7.35 (d, 2H), 7.00 (d, 2H), 4.65 (m, 1H), 4.20 (m, 1H), 3.80 (m, 1H), 3.35 (s, 3H), 2.80 to 2.10 (m, 6H).

### DP Example 6 Method-1

### [9-[(3,4-Dichlorophenyl)thiol-1-(methylsulfonyl)-7,8-dihydro-6H-pyrido[3,4-b]pyrrolizin-8-yl]acetic acid (Compound L)

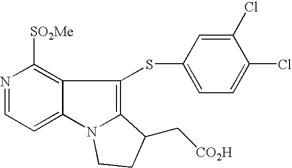

### Step 1 2-(Methylthio)nicotinaldehyde

The title compound was prepared from 2-bromonicotinaldehyde (A. Numata Synthesis 1999 p.306) as described in Example 1 Step 2 except the solution was heated at 55° C. for 2 hr.

### Step 2 Methyl (2Z)-2-azido-3-[2-(methylthio)pyridin-3-yl]prop-2-enoate

The title compound was prepared as described in Example 1 Step 3.

### Step 3 Methyl 4-(methylthio)-1H-pyrrolo[3,2-c]pyridine-2-carboxylate

A solution of methyl (2Z)-2-azido-3-[2-(methylthio)pyridin-3-yl]prop-2-enoate (1.00 g, 4.00 mmol) in mesitylene (50 mL) was heated at 160° C. for a period of 1 h. The reaction mixture was cooled to room temperature then to 0° C. , the precipitate was filtered and washed with cold mesitylene to provide the title compound.

### Step 4 Methyl 1-(methylthio)-8-oxo-7,8-dihydro-6H-pyrido[3,4-b]pyrrolizine-7-carboxylate

To a suspension of methyl 4-(methylthio)-1H-pyrrolo[3,2-c]pyridine-2-carboxylate (0.30 g, 1.35 mmol) in THF (3 mL)-toluene (12.0 mL) were added a 1.06 M THF solution of potassium tert-butoxide (1.42 mL/1.41 mmol)and methyl acrylate (300 μL). The resulting mixture was heated at 80° C. for 18 h. The mixture was partitioned between EtOAc and NH₄Cl, and filtered through Celite. The organic phase was separated, dried over Na₂SO₄ and filtered, to provide the title compound.

### Step 5 1-(Methylthio)-6,7-dihydro-8H-pyridor3,4-b]pyrrolizin-8-one

Methyl 1-(methylthio)-8-oxo-7,8-dihydro-6H-pyrido[3,4-b]pyrrolizine-7-carboxylate was converted to the title compound as described in Example 1 Step 6.

### Step 6 Methyl [8-hydroxy-1-(methylthio)-7,8-dihydro-6H-pyridor3,4-b]pyrrolizin-8-yl]acetate

A mixture of 1-(methylthio)-6,7-dihydro-8H-pyrido[3,4-b]pyrrolizin-8-one (0.15 g, 0.68 mmol), methyl bromoacetate (0.34 mL), Zn—Cu (0.226 g) in THF (3.0 mL) was sonicated for 2 h. The mixture was then heated at 60° C. for 5 min. until completion of the reaction. The reaction mixture was partitioned between EtOAc and NH₄Cl. The organic phase was separated, dried over Na₂SO₄, filtered and evaporated under reduced pressure to provide the title compound. The compound was purified by flash chromatography.

### Step 7 Methyl [1-(methylthio)-7,8-dihydro-6H-pyrido[3 4-b]pyrrolizin-8-yl]acetate

To NaI (0.300 g) in CH₃CN (3.2 mL) was added TMSCl (0.266 mL). This mixture was added to a suspension of methyl [8-hydroxy-1-(methylthio)-7,8-dihydro-6H-pyrido[3,4-b]pyrrolizin-8-yl]acetate (0.15 g, 0.515 mmol) in CH₃CN (1.5 mL), in a water bath. After a period of 0.5 h, the reaction mixture was partitioned between EtOAc and NaHCO₃. The organic phase was separated, washed with sodium thiosulphate, dried over MgSO₄ and evaporated. The title compound was purified by flash chromatography.

### Step 8 Methyl [1-(methylsulfonyl)-7,8-dihydro-6H-pyridor3,4-b]pyrrolizin-8-yl]acetate

Methyl [1-(methylthio)-7,8-dihydro-6H-pyrido[3,4-b]pyrrolizin-8-yl]acetate was converted to the title compound as described in Example 1 Step 9.

### Step 9 [9-[(3,4-Dichlorophenyl)thiol-1-(methylsulfonyl)-7,8-dihydro-6H pyrido[3,4-b]pyrrolizin-8-yL]acetic acid

Methyl [1-(methylsulfonyl)-7,8-dihydro-6H-pyrido[3,4-b]pyrrolizin-8-yl]acetate was converted to the title compound as described in Example 1, Steps 10 and 11, using bis (3,4-dichlorophenyl)disulfide in Step 10.

¹H NMR (500 MHz, acetone-d₆) δ 8.35 (d, 1H) 7.80 (d, 1H), 7.35 (d, 1H), 7.15 (s, 1H), 6.95 (d, 1H), 4.55 (m, 1H), 4.35 (m, 1H), 3.90 (m, 1H), 3.30 (s, 3H), 3.15 (m, 1H), 3.05 (m, 1H), 2.80 (m, 1H), 2.50 (m, 1H).

### DP Example 6 Method-2

### [9-[(3,4-Dichlorophenyl)thiol]-1-(methylsulfonyl)-7,8-dihydro-6H-pyrido3,4-b]pyrrolizin-8-yl]acetic acid

### Step 1 1-(Methylthio)-7,8-dihydro-6H-pyrido[3.4-b]pyrrolizin-8-ol

To a suspension of 1-(methylthio)-6,7-dihydro-8H-pyrido[3,4-b]pyrrolizin-8-one from Example 6, Method-1 Step 5 (0.55 g, 2.2 mmol) in EtOH (10 mL)-THF (1 mL) was added NaBH₄ (0.10 g, 2.6 mmol) at 0° C. After a period of 30 min. at room temperature, the reaction was quenched by the addition of acetone. The solvents were evaporated under reduced pressure and EtOAC and H₂O were added to the residue. The organic phase was separated, dried over MgSO₄ and evaporated. The title compound was washed with EtOAc/Hexane and filtered.

### Step 2 Dimethyl 2-[1-(methylthio)-7,8-dihydro-6H-pyrido[3,4-b]pyrrolizin-8-yl]malonate

To a suspension of 1-(methylthio)-7,8-dihydro-6H-pyrido[3,4-b]pyrrolizin-8-ol (0.54 g, 2.1 mmol) in THF (10 mL) at −78° C. were added 1M NaHMDS in THF (2.35 mL, 2.4 mmol) and diphenyl chlorophosphate (0.53 mL, 2.6 mmol). After a period of 30 min. dimethyl malonate (0.73 mL, 6.4 nmmol) and 1M NaHMDS in THF (6.8 mL, 6.8 mmol) were added. The reaction mixture was brought to 0° C. and then to room temperature. The mixture was then partitioned between ETOAc and NH₄Cl. The organic phase was dried over MgSO₄, filtered and evaporated. The title compound was purified by flash chromatography.

### Step 3 Methyl [1-(methylthio)-7,8-dihydro-6H-pyridor[3,4-b]pyrrolizin-8-yl]-acetate

To a mixture of dimethyl 2-[1-(methylthio)-7,8-dihydro-6H-pyrido[3,4-b]pyrrolizin-8-yl]malonate (0.59 g, 2.17 mmol) and DMSO (4 mL) was added NaCl (0.45 g) in H₂O (0.45 mL). After a period of 18 h at 150° C., the reaction mixture was partitioned between ETOAc and H₂O. The organic phase was separated, dried over Na₂SO₄ and evaporated. The title compound was then purified by flash chromatography.

### Step 4 [9-[(3,4-Dichlorophenyl)thiol-1-(methylsulfonyl)-7,8-dihydro-6H-pyrido[3,4-b]pyrrolizin-8-yl]acetic acid

The title compound was obtained from methyl [1-(methylthio)-7,8-dihydro-6H-pyrido[3,4-b]pyrrolizin-8yl]acetate as described in Example 6, Method-1, Steps 8 to 9.

### DP Example 7

### [10-[(3,4-Dichlorophenyl)sulfanyl]-1-(methylsulfonyl)-6,7,8,9-tetrahydropyrido3,4-b]indolizin-9-yl]acetic acid (Compound M)

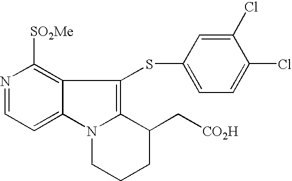

### Step 1 Ethyl[1-(methylsulfonyl)-6,7,8,9-tetrahydropyrido[3,4-b]indolizin-9-yl]acetate

The title compound was prepared from the product of Example 6, Step 3 in the same manner as described in Example 1, Steps 5 to 9.

### Step 2 [10-[(3,4-Dichlorophenyl)sulfanyl]-1-(methylsulfonyl)-6,7,8,9-tetrahydropyrido[3,4-b]indolizin-9-yl]acetic acid

The product of Step 1 was converted to the title compound in the same manner as Example 1, Steps 10-11, using bis (3,4-dichlorophenyl)disulfide in Step 10.

MS M+1=485.

### DP Example 8

### (4-(Methylsulfonyl)-5-{4-(trifluoromethyl)phenyl]thio}-6,7,8,9-tetrahydropyrido[3,2-b]indolizin-6-yl)acetic acid (Compound N)

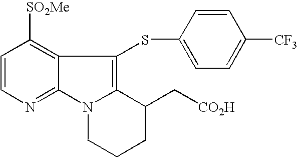

The title compound was prepared as described in Example 1 using bis[4-trifluoromethyl)phenyl]disulfide.

¹H NMR (500 MHz, acetone-d₆) δ 8.55 (d, 1H), 7.75 (d, 1H), 7.45 (d, 2H), 7.15 (d, 2H), 4.55 (m, 1H), 4.15 (m, 1H), 3.80 (m, 1H), 3.30 (s, 3H), 2.80 to 2.10 (m, 6H). m/z 513 (M+1).

### DP Example 9

### [5-[(2-Chloro-4-fluorophenyl)thio]-4-(methylsulfonyl)-6,7,8,9-tetrahydropyrido[3,2-b]indolizin-6-yl]acetic acid (Compound O)

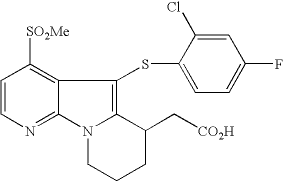

The title compound was prepared as described in Example 1 using bis(2-chloro-4-fluorophenyl)disulfide.

m/z 469 (M+1). DP Example 10

### [4-(Methylsulfonyl)-5-(2-naphthylthio)-6,7,8,9-tetrahydropyrido[3,2-b]indolizin-6-yl]acetic acid (Compound P)

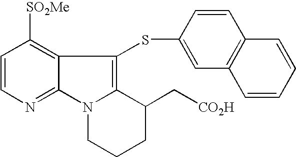

The title compound was prepared as described in Example 1 using di(2-naphthyl) disulfide.

M/z 467 (M+1).

### DP Example 11

### [5-[(2,3-Dichlorophenylthio]-4-(methylsulfonyl)-6,7,8,9-tetrahydropyrido[3,2-b]indolizin-6-yl]acetic acid (Compound Q)

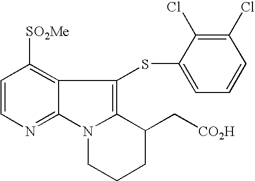

The title compound was prepared as described in Example 1 using bis(2,3-dichlorophenyl)disulfide.

¹H NMR (500 MHz, acetone-d₆) δ 8.85 (d, 1H), 7.80 (d, 1H), 7.30 (d, 1H), 7.00 (t, 1H), 6.60 (d, 1H), 4.60 (m, 1H), 4.20 (m, 1H), 3.80 (m, 1H), 3.40 (s, 3H), 2.80 to 2.10 (m, 6H).

### DP Example 12

### [5-[(4-Methylphenyl)thio]-4-(methylsulfonyl)-6,7,8,9-tetrahydropyrido[3,2-b]indolizin-6-yl]acetic acid (Compound R)

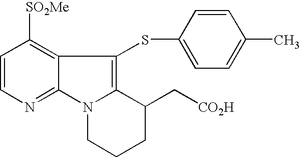

The title compound was prepared as described in Example 1 using p-tolyl disulfide.

¹H NMR (500 MHz, acetone-d₆) δ 8.55 (d, 1H), 7.80 (d, 1H), 6.95 (m, 4H), 4.60 (m, 1H), 4.15 (m, 1H), 3.80 (m, 1H), 3.35 (s, 3H), 2.80 to 2.10 (m, 6H).

### DP Example 13

### [4-(Methylsulfonyl)-5-(phenylthio)-6,7,8,9-tetrahydropyrido[3,2-b]indolizin-6-yl]acetic acid (Compound S)

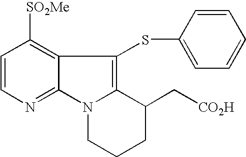

The title compound was prepared as described in Example 1 using diphenyl disulfide.

¹H NMR (500 MHz, acetone-d₆) δ 8.55 (d, 1H), 7.80 (d, 1H), 7.15 to 6.90 (m, 5H), 4.60 (m, 1H), 4.15 (m, 1H), 3.75 (m, 1H), 3.30 (s, 3H), 2.80 to 2.10 (m, 6H).

### DP Example 14

### [5-[(2,4-Dichlorophenyl)thio]-4-methylsulfonyl)-6,7,8,9-tetrahydropyrido[3,2-b]indolizin-6-yl]acetic acid (Compound T)

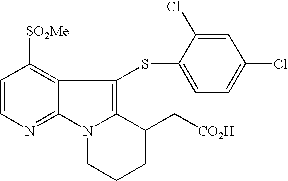

The title compound was prepared as described in Example 1 using bis(2,4-dichlorophenyl)disulfide. The disulfide was prepared from 2,4-dichlorothiophenyl using Br₂ in ether.

¹H NMR (500 MHz, acetone-d₆) δ 8.55 (d,1H), 7.85 (d, 1H), 7.35 (s, 1H), 7.00 (d, 1H), 6.65 (d, 1H), 4.55 (m, 1H), 4.15 (m, 1H), 3.80 (m, 1H), 3.35 (s, 3H), 2.80 to 2.10 (m, 6H).

### DP Example 15

### [5-[(4-Chlorophenylthio]-4-(methylsulfonyl)-6,7,8,9-tetrahydropyido[4,3-b]indolizin-6-yl]acetic acid (Compound U)

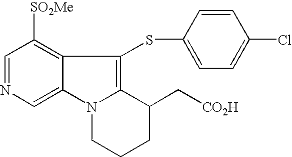

The title compound was prepared as described in Example 1 from 3-chloronicotinaldehyde (Heterocycles p. 151, 1993) except the terminal cyclization was performed by adding the azide to decalin at reflux.

¹H NMR (500 MHz, acetone-d₆) δ 9.20 (s, 1H), 8.85 (s, 1H), 7.20 (d, 2H), 7.00 (d, 2H), 4.70 (m, 1H), 4.30 (m, 1H), 3.75 (m, 1H), 3.35 (s, 3H), 2.80 to 2.10 (m, 6H).

### DP Example 16

### [9-[(4-Chlorophenyl)thio]-1-(methylsulfonyl)-7,8-dihydro-6H-pyrido[3,4-b]pyrrolizin-8-yl]acetic acid (Compound V)

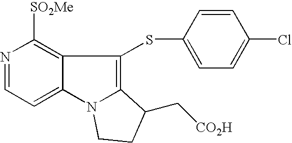

The title compound was prepared from the product of Example 6 Method 1 Step 8, as described in the procedures outlined in Example 1 Steps 10 and 11, using bis (4-chlorophenyl)disulfide in Step 10.

¹H NMR (500 MHz, acetone-d₆) δ 8.25-8.3 (m, 1H), 7.71-7.75 (m, 1H), 7.12-7.17 (m, 2H), 6.97-7.04 (m, 2H), 4.45-4.51 (m, 1H), 4.32-4.39 (m, 1H), 3.73-3.80 (m, 1H), 3.29 (s, 3H), 3.15-3.21 (m, 1H), 2.99-3.08 (m, 1H), 2.66-2.73 (m, 1H), 2.46-2.54 (m, 1H).

### DP Example 17

### (−)-[(4-Chlorobenzyl)-7-fluoro-5-methanesulfonyl)-1,2,3,4-tetrahydrocyclopenta[b]indol-3-yl]acetic acid (Compound E)

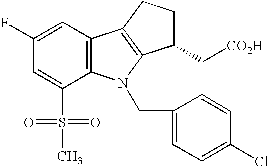

### Step 1: (±)-(7-Fluoro-1,2,3,4-tetrahydrocyclopenta[b]indol-3-yl)acetic acid ethyl ester.

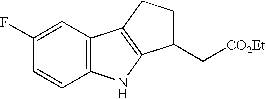

A solution of 10.00 g of 4-fluoro-2-iodoaniline, 6.57 g of ethyl 2-(2-oxocyclopentyl)acetate and 121 mg of p-toluenesulfonic acid in 100 ml of benzene was refluxed with a Dean-Stark trap under a N₂ atmosphere for 24 h. After this time, the benzene was removed under distillation. Then, 60 ml of DMF was added and the solution was degassed before 19 ml of Hunig's base followed by 405 mg of Pd(OAc)₂ were added successively. The solution was heated to 115° C. for 3 h, then cooled to room temperature. To quench the reaction, 300 ml of 1 N HCl and 200 ml of ethyl acetate were added and the mixture was filtered through Celite. The phases were separated and the acidic phase was extracted twice with 200 ml of ethyl acetate. The organic layers were combined, washed with brine, dried over anhydrous Na₂SO₄, filtered through Celite and concentrated. The crude material was further purified by flash chromatography eluting with 100% toluene, to provide the title compound.

¹H NMR (acetone-d₆) δ 9.76 (br s, 1H), 7.34 (dd, 1H), 7.03 (d, 1H), 6.78 (td, 1H), 4.14 (q, 2H), 3.57 (m, 1H), 2.85-2.55 (m, 5H), 2.15 (m, 1H), 1.22 (t, 3H).

### Step 2: (±)-(7-Fluoro-1,2,3,4-tetrahydrocyclopenta[b]indol-3-yl)acetic acid

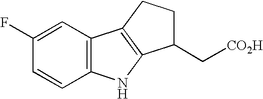

To a solution of 1.24 g of the ester from Step 1 in 14 mL of tetrahydrofuran (THF) at room temperature, 7 mL of MeOH followed by 7 mL of 2N NaOH were added. After 2.5 h, the reaction mixture was poured into a separatory funnel containing ethyl acetate (EtOAc)/1N HCl. The phases were separated and the acidic phase was extracted twice with EtOAc. The organic layers were combined, washed with brine, dried over anhydrous Na₂SO₄ and evaporated to dryness to yield a crude oil that was used as such in the next step (>90% purity).

¹H NMR (acetone-d₆) δ 10.90 (br s, 1H), 9.77 (br s, 1H), 7.34 (dd, 1H), 7.04 (dd, 1H), 6.79 (td, 1H), 3.56 (m, 1H), 2.90-2.50 (m, 5H), 2.16 (m, 1H). MS (−APCI) m/z 232.2 (M−H)⁻.

### Step 3: (±)-(5-bromo-7-fluoro-1,2,3,4-tetrahydrocyclopenta[b]indol-3-yl)acetic acid

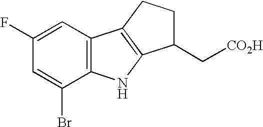

To a solution of 2.20 g of the acid from Step 2 (>90% purity) in 30 mL of pyridine, 6.85 g of pyridinium tribromide (90% purity) was added at 40° C. The suspension was stirred for 10 min at 0° C. and warmed to room temperature for 30 min. Then, the solvent was removed without heating under high vacuum. The crude material was dissolved in 40 mL of AcOH and 2.88 g of Zn dust was added portion wise to the cold solution at 0° C. The suspension was stirred for 15 min at 15° C. and warmed to room temperature for an additional 15 min. At this time, the reaction mixture was quenched by the addition of 1N HCl and this mixture was poured into a separatory funnel containing brine/EtOAc. The layers were separated and the organic layer was washed with water, brine, dried over anhydrous Na₂SO₄ and concentrated. This material was used without further purification in the next step.

¹H NMR (acetone-d₆) δ 10.77 (br s, 1H), 9.84 (br s, 1H), 7.09 (m, 2H), 3.60 (m, 1H), 2.95-2.65 (m, 4H), 2.56 (dd, 1H), 2.19 (m, 1H).

### Step 4: (±)-[5-bromo-4-(4-chlorobenzyl)-7-fluoro-1,2,3,4-tetrahydrocyclopenta[b]indol-3-yl]-acetic acid

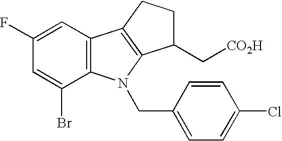

To a solution of 2.13 g of the acid from Step 3 in 10 mL of THF, a solution of diazomethane in ether was added in excess until complete consumption of the acid as monitored on TLC. Then, the solvents were removed under vacuum. To a solution of the crude methyl ester thus formed in 20 mL of DMF, 539 mg of a NaH suspension (60% in oil) was added at −78° C. The suspension was stirred for 10 min at 0° C., cooled again to −78° C. and treated with 1.70 g of 4-chlorobenzyl bromide. After 5 min, the temperature was warmed to 0° C. and the mixture was stirred for 20 min. At this time, the reaction was quenched by the addition of 2 mL of AcOH and this mixture was poured into a separatory funnel containing 1N HCl/EtOAc. The layers were separated and the organic layer was washed with brine, dried over anhydrous Na₂SO₄ and concentrated. The alkylated material was hydrolyzed using the procedure described in Step 2. The crude material was further purified by trituration with EtOAc/hexanes to provide the title compound.

¹H NMR (acetone-d₆) δ 10.70 (br s, 1H), 7.31 (d, 2H), 7.18 (d, 1H), 7.06 (d, 1H), 6.92 (d, 2H), 5.90 (d, 1H), 5.74 (d, 1H), 3.61 (m, 1H), 3.00-2.70 (m, 3H), 2.65 (dd, 1H), 2.39 (dd, 1H), 2.26 (m, 1H). MS (−APCI) m/z 436.3, 434.5 (M−H)⁻.

### Step 5: (+)-[5-bromo-4-(4-chlorobenzyl)-7-fluoro-1,2,3,4-tetrahydrocyclopenta[b]indol-3- yl}acetic acid

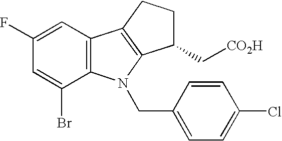

To a solution of 2.35 g of the acid of Step 4 in 130 mL of EtOH at 80° C., was added 780 μL of (S)-(−)-1-(1-naphthyl)ethylamine. The solution was cooled to room temperature and stirred overnight. The salt recovered (1.7 g) was recrystallized again with 200 mL of EtOH. After filtration, the white solid salt obtained was neutralized with 1N HCl and the product was extracted with EtOAc. The organic layer was washed with brine, dried over anhydrous Na₂SO₄ and concentrated. The material was filtered over a pad of SiO₂ by eluting with EtOAc to produce the title enantiomer. Retention times of the two enantiomers were respectively 7.5 min and 9.4 min [ChiralPak AD column, hexane/2-propanol/acetic acid (95:5:0.1)]. The more polar enantiomer was in 98% ee.

ee=98%; Retention time=9.4 min [ChiralPak AD column: 250×4.6 mm, hexanes/2-propanol/acetic acid (75:25:0.1)]; [α]_{D}²¹=+39.20 (c 1.0, MeOH).

### Step 6: (−)-[4-(4-chlorobenzyl)-7-fluoro-5-(methanesulfonyl)-1,2,3 4-tetrahydrocyclopenta[b]-indol-3-yl}acetic acid and sodium salt

The acid from Step 5 (15.4 g) was first esterified with diazomethane. The sulfonylation was accomplished by mixing the ester thus formed with 16.3 g of methanesulfinic acid sodium salt and 30.2 g of CuI (I) in N-methylpyrrolidinone. The suspension was degassed under a flow of N₂, heated to 150° C. and stirred for 3 h, then cooled to room temperature. To quench the reaction, 500 ml of ethyl acetate and 500 ml of hexanes were added and the mixture was filtered through a pad of SiO₂ by eluting with EtOAc. The organic phases were concentrated. The crude oil was dissolved with EtOAc, washed three times with water one time with brine, dried over anhydrous Na₂SO₄, filtered and concentrated. The crude material was further purified by flash chromatography eluting with a gradient from 100% toluene to 50% toluene in EtOAc, to provide 14 g of the sulfonated ester, which was hydrolyzed using the procedure described in Step 2. The title compound was obtained after two successive recrystallizations: isopropyl acetate/heptane followed by CH₂Cl₂/hexanes.

¹H NMR (500 MHz acetone-d₆) δ 10.73 (br s, 1H), 7.57 (d, 2H, J=8.8 Hz), 7.31 (m, 1H), 7.29 (m, 1H), 6.84 (d, 2H, J=8.8 Hz), 6.29 (d, 1H, J_{AB}=17.8 Hz), 5.79 (d, 1H, J_{AB}=17.8 Hz), 3.43 (m, 1H), 2.98 (s, 3H), 2.94 (m, 1H), 2.85-2.65 (m, 3H), 2.42 (dd, 1H, J₁=16.1 Hz, J₂=10.3 Hz), 2.27 (m, 1H). ¹³C NMR (125 MHz acetone-d₆) δ 173.0, 156.5 (d, J_{CF}=237 Hz), 153.9, 139.2, 133.7, 133.3, 130.0 (d, J_{CF}=8.9 Hz), 129.6, 128.2, 127.5 (d, J_{CF}=7.6 Hz), 122.2 (d, J_{CF}=4.2 Hz), 112.3 (d, J_{CF}=29.4 Hz), 111.0 (d, J_{CF}=22.6 Hz), 50.8, 44.7, 38.6, 36.6, 36.5, 23.3. MS (−APCI) m/z 436.1, 434.1 (M−H)⁻.

ee=97%; Retention time=15.3 min [ChiralCel OD column: 250×4.6 mm, hexanes/2-propanol/ethanol/acetic acid (90:5:5:0.2)]; [α]_{D}²¹=−29.3° (c 1.0, MeOH). Mp 175.0° C.

The sodium salt was prepared by the treatment of 6.45 g (14.80 mmol) of the above acid compound in EtOH (100 mL) with 14.80 mL of an aqueous 1N NaOH solution. The organic solvent was removed under vacuum and the crude solid was dissolved in 1.2L of isopropyl alcohol under reflux. The final volume was reduced to 500 mL by distillation of the solvent. The sodium salt crystallized by cooling to rt. The crystalline sodium salt was suspended in H₂O, frozen with a dry ice bath and lyophilized under high vacuum to give the title compound as the sodium salt.

¹H NMR (500 MHz DMSO-d₆) δ 7.63 (dd, 1H, J₁=8.5 Hz, J₂=2.6 Hz), 7.47 (dd, 1H, J₁=9.7 Hz, J₂=2.6 Hz), 7.33 (d, 2H, J=8.4 Hz), 6.70 (d, 2H, J=8.4 Hz), 6.06 (d, 1H, J_{AB}=17.9 Hz), 5.76 (d, 1H, J_{AB}=17.9 Hz), 3.29 (m, 1H), 3.08 (s, 3H), 2.80 (m, 1H), 2.69 (m, 1H), 2.55 (m, 1H), 2.18 (m, 2H), 1.93 (dd, 1H, J₁=14.4 Hz, J₂=9.7 Hz).

### DP Example 17A

### Alternative procedure for (±)- [5-bromo-4-(4-chlorobenzyl)-7-fluoro-1,2,314- tetrahydrocyclopenta[b]indol-3-yl]acetic acid (Example 17, Step 4)

### Step 1: (±)-7-fluoro-1,2,3,4-tetrahydrocyclopenta[b]indol-3-yl)acetic acid dicyclohexylamine (DCHA) salt

A 0.526 M solution of 2-bromo-4-fluoroaniline in xylene along with ethyl (2-oxocyclopentyl) acetate (1.5 eq) and sulfuric acid (0.02 eq) was heated to reflux for 20 hours. Water was azeotropically removed with a Dean-Stark apparatus. The reaction was followed by NMR and after 20 hours, an 80-85% conversion to the desired imine intermediate was generally observed. The reaction mixture was washed with 1M sodium bicarbonate (0.2 volumes) for 15 minutes and the organic fraction was evaporated. The remaining syrup was distilled under vacuum (0.5 mm Hg). Residual xylenes distilled at 30° C., then excess ketone and unreacted aniline were recovered in the 50-110° C. range; the imine was recovered in the 110-180° C. fraction as a light brown clear liquid with 83% purity.

The imine intermediate was then added to a degased mixture of potassium acetate (3 eq), tetra-n-butylanmmonium chloride monohydrate (1 eq), palladium acetate (0.03 eq) and N,N-dimethylacetamide (final concentration of imine=0.365 M). The reaction mixture was heated to 115° C. for 5 hours and allowed to cool to room temperature. 3N KOH (3 eq) was then added and the mixture was stirred at room temperature for 1 hour. The reaction mixture was diluted with water (1.0 volume), washed with toluene (3×0.75 volume). The aqueous phase was acidified to pH 1 with 3N HCl and extracted with tertbutyl methyl ether (2×0.75 volume). The combined organic fractions were washed with water (0.75 volume). To the clear light brown solution was added dicyclohexylamine (1 eq) and the solution was stirred at room temperature for 16 hours. The salt was filtered, washed with ethyl acetate, tertbutyl methyl ether and allowed to dry to give the title compound. Assay: 94 A %.

¹H NMR (500 mHz, CDCl3): δ 9.24 (s, 1H), 7.16-7.08 (m, 2H), 6.82 (t, 1H), 6.2 (br, 2H), 3.6-3.5 (m, 1H), 3.04-2.97 (m, 2H), 2.88-2.70 (m, 3H), 2.66 (dd, 1H), 2.45-2.37 (m, 1H), 2.13-2.05 (m, 2.05), 1.83 (d, 4H), 1.67 (d, 2H), 1.55-1.43 (m, 4H), 1.33-1.11 (m, 6H).

### Step 2: (±)-(5-bromo-7-fluoro-1 2,3 4-tetrahydrocyclopenta[b]indol-3-yl)acetic acid

A slurry of the DCHA salt from Step 1 above in dichloromethane (0.241 M solution) was cooled to −20 to −15° C. Pyridine (2 eq.) was added in one shot and to the slurry was added dropwise bromine (2.5 eq.) over 30 to 45 minutes maintaining the temperature between −20° C. and −15° C. (At about ⅓ addition of bromine, the reaction mixture was thick and an efficient stirring was needed. Eventually, at about ½ addition of bromine, the mixture became “loose” again.) After completion of the addition, the reaction mixture was aged for one additional hour at −15° C. Acetic acid (3.04 eq.) was then added over 5 minutes and zinc dust (3.04 eq.) was added portion wise. (A portion of zinc was added at −15° C. and the mixture was aged for about 5 minutes to ensure that the exotherm was going (about −15 ° C. to −10° C.)). This operation was repeated with about 5 shots of zinc over about 30 min. When no more exotherm was observed, the remaining zinc was added faster. The whole operation took around 30 to 45 minutes.

After completion of the addition, the batch was warmed to room temperature, aged 1 hour and concentrated. The reaction mixture was switched to methyl t-butyl ether (MTBE, 0.8 volume) and a 10% aqueous acetic acid solution (0.8 volume) was added. The mixture (crystallization of salts, e.g pyridium) was aged at room temperature for 1 hour and filtered through solka-floc. The pad of solka-floc was rinsed with MTBE (ca. 0.2 volume) and the filtrate (biphasic, MTBE/aqueous) was transferred into an extractor. The organic phase was washed with water (0.8 volume). The MTBE extract was concentrated and switched to isopropyl alcohol (IPA, 0.25 volume) to crystallize the compound. Water (0.25 volumes) was added and the batch was aged for 1 hour. Additional water (0.33 volumes) was added over 1 hour. After completion of the water addition, the batch was aged for one additional hour, filtered, and rinse with 30/70 IPA/Water (0.15 volumes). Crystallized bromoacid was dried in the oven at +45° C.

### Step 3: (±)-[5-bromo-4-(4-chlorobenzyl)-7-fluoro-1,2,3 4-tetrahydrocyclopenta[b]indol-3-yl]-acetic acid

The bromoacid of Step 2 was dissolved in dimethylacetamide (0.416 M solution) and cesium carbonate (2.5 eq.) was added in one portion. To the slurry was added in one portion 4-chlorobenzyl chloride (2.5 eq.) and the batch was heated to 50° C. for 20 h. The batch was cooled to r.t. and sodium hydroxide 5N (4.00 eq.) was added over 5 minutes (temperature rose to +40° C.). The reaction was aged at 50° C. for ca. 3 hours, cooled to room temperature and transferred into an L extractor. The solution was diluted with isopropylacetate (IPAc, 2 volumes) and cooled to +15° C. The solution was acidified with 5N HCl to pH˜2. Layers were separated and the organic layer was washed with water (2×2 volumes). IPAc solution was concentrated and switched to IPA (0.8 volumes) to crystallize the product. Water (8 L) was added over 2 hours and the batch was filtered to give the title compound. The batch can be dried in the oven at +40° C. for 24 hours.

### DP Example 18

### (±) -{4-[1-(4-Chlorophenyl)ethyl]-7-fluoro-5-methanesulfonyl-1,2,3,4-tetrahydrocyclopenta[b]indol-3-yl}acetic acid(Compound X)

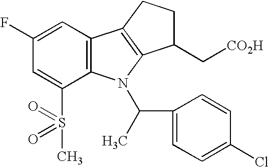

The title compound was synthesized in accordance with the description provided in PCT WO03/062200 published on Jul. 30, 2003.

### DP Example 19

### (±)-[9-(4-Chlorobenzyl)-6-fluoro-methanesulfonyl -2,3,4,9-tetrahydro-1H-carbazol-1-yl]acetic acid (Compound Y)

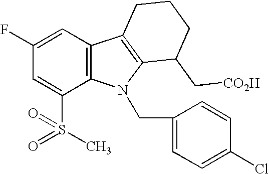

The title compound was synthesized in accordance with the description provided in PCT WO03/062200 published on Jul. 30, 2003.

### DP Example 20

### [4-(4-Chlorobenzyl)-7-fluoro-5-methanesulfonyl-1-oxo-1,2,3,4-tetrahydrocyclopenta[b]indol-3-yl]acetic acid (Compound Z)

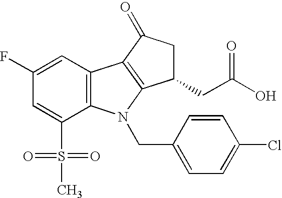

The title compound was synthesized in accordance with the description provided in PCT WO03/062200 published on Jul. 30, 2003.

### DP Example 21

### {9-[(3 4-Dichlorophenyl)thiol-1-isopropyl-7,8-dihydro-6H-pyrido[3,4-b]pyrrolizin-8-yl}acetic acid (Enantiomer A and Enantiomer B) (Compound AA)

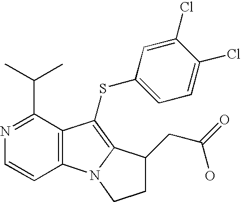

### Step 1 2-Chloronicotinaldehyde

To a solution of diisopropyl amine (110 mL, 780 nunol) in THF (500 mL) was added a 2.5 M hexanes solution of n-BuLi (300 mL, 750 mmol) at −40° C. After 5 min, the reaction mixture was cooled to −95° C. then DMPU (15 mL) and 2-chloropyridine (50 mL, 532 mmol) were successively added. The resulting mixture was then warmed and stirred at −78° C. for 4 h. After this time, the yellow suspension was cooled again to −95° C. before DMF (70 mL) was added. The final reaction mixture was warmed to −78° C. and stirred at that temperature for 1.5 h. The reaction mixture was poured into cold aqueous HCl (3N, 800 mL) and stirred for 5 min. Aqueous concentrated NH₄OH was added to adjust pH to 7.5. The aqueous layer was extracted three times with EtOAc. The combined organic layer was washed with aqueous NH₄Cl and brine, dried over anhydrous Na₂SO₄, filtered and concentrated. The crude material was further purified by a pad of silica gel by eluting with a gradient from 100% hexanes to 100% EtOAc and the product was crystallized in cold hexanes to yield the title compound as a pale yellow solid.

### Step 2 Methyl (2Z)-2-azido-3-(2-chloropyridin-3-yl)prop-2-enoate

A solution of 2-chloronicotinealdehyde (20.0 g, 139.9 mmol) and methyl azidoacetate (32.2 mL, 349.7 mmol) in MeOH (168 mL) was added to a solution of 25% NaOMe in MeOH (80 mL, 349 mmol) at −20° C. The internal temperature was monitored and maintained at −20° C. during the 30 min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum. The crude material was dissolved in CH₂Cl₂ and MgSO₄ was added. The suspension was filtered through a pad of silica gel, washed with CH₂Cl₂. The filtrate was concentrated under reduced pressure and a beige precipitate (20 g) of the title product was obtained.

### Step 3 Methyl 4-chloro-1H-pyrrolo[3,2-c]pyridine-2-carboxylate

A solution of methyl (2Z)-2-azido-3-[2-chloropyridin-3-yl]prop-2-enoate (21 g, 88 mmol) in mesitylene (880 mL) was heated at reflux for a period of 1 h. The reaction mixture was cooled to room temperature then to 0° C., and the precipitate was filtered and washed with cold hexane. The material was stirred overnight in 1:20 EtOAc/hexane to give, after filtration, the title product as a pale yellow solid (13.2 g).

### Step 4 Methyl 1-chloro-8-oxo-7,8-dihydro-6H- prido[3,4-b]pyrrolizine-7-carboxylate

To a suspension of methyl 4-chloro-1H-pyrrolo[3,2-c]pyridine-2-carboxylate (12.5 g, 59 mmol) in THF (116 mL)—toluene (460 mL) were added a 1.0 M THF solution of potassium tert-butoxide (64 mL, 64 mmol) and methyl acrylate (55 mL, 611 mmol). The resulting mixture was heated at 100° C. for 18 h. After this time, the suspension was cooled to room temperature and it was poured into a mixture of saturated aqueous NH₄Cl (400 mL) and hexanes (400 mL). The solids were decanted, filtered and washed with H₂O and hexanes to provide the title compound.

### Step 5 1-Chloro-6,7-dihydro-8H-pyrido[3,4-b]pyrrolizin-8-one

To the compound of the previous step were added isopropanol (8.0 mL) and concentrated HCl (2.0 mL) with heating at 100° C. for 1 h. The reaction mixture was partitioned between EtOAc and Na₂CO₃. The organic phase was separated, evaporated to provide the title compound.

### Step 6 1-Isopropenyl-6,7-dihydro-8H-pyrido[3,4-b]pyrrolizin-8-one

To a mixture of 1-chloro-6,7-dihydro-8H-pyrido[3,4-b]pyrrolizin-8-one (5.0 g, 24.3 mmol), tris (dibenzylidene acetone)dipalladium (0) (1.0 g, 1.09 mmol) and triphenylarsine (2.70 g, 8.82 mmol) in DMF (100 mTL) was added tributylisopropenyl stannane (9.60 g, 29.00 mmol). The resulting mixture was degassed and heated at 78° C. for a period of 18 h. The solvent was evaporated under reduced pressure. CH₂Cl₂ and celite were added to the resulting mixture which was then filtered over celite. The title compound was purified by flash chromatography (50% to 100% EtOAc in Hexane).

### Step 7 Ethyl (2E)-(1-isopropenyl-6,7-dihydro-8H-pyrido[3,4-b]pyrrolizin-8-ylidene)ethanoate

To a solution of 1-isopropenyl-6,7-dihydro-8H-pyrido[3,4-b]pyrrolizin-8-one (0.60 g, 2.8 mmol) and triethyl phosphonoacetate (1.00 g, 4.46 mmol) in THF (24 mL) at −78° C. was added 80% NaH (0.12 g, 4.00 mmol), the reaction mixture was allowed to warm to 0° C., then to room temperature. The reaction mixture was poured onto saturated NH₄Cl and EtOAc. The organic phase was separated, dried over Na₂SO₄ and evaporated. The title compound was purified by flash chromatography (40% EtOAc in Hexane).

### Step 8 Ethyl (1-isopropyl-7,8-dihydro-6H-pfrido[3,4-b]pyrrolizin-8-yl)acetate

To a solution of ethyl (2E)-(1-isopropenyl-6,7-dihydro-8H-pyrido[3,4-b]pyrrolizin-8-ylidene)ethanoate (0.40 g, 1.4 mmol) in MeOH (20 mL) was added Pd(OH)₂ (0.20 g). The mixture was stirred under 1 atm of H₂ for 3 h. The mixture was filtered over celite and evaporated to provide the title compound.

### Step 9 Ethyl {9-[(3 4-dichlorophenyl)thiol-1-isopropyl-7,8-dihydro-6H-pyrido [3,4-b]pyrrolizin-8-yl]acetate

To a solution of bis (3,4-dichlorophenyl)disulfide (0.24 g, 0.67 mmol) in CH₂Cl₂ (5.6 mmL) was added SO₂Cl₂ (0.036 mL). The resulting yellow mixture was stirred at room temperature for 1 h. This solution was added to a solution of ethyl (1-isopropyl-7,8-dihydro-6H-pyrido[3,4-b]pyrrolizin-8-yL) acetate (0.15 g, 0.52 mmol) in DMF (5.6 mL) at 0° C. After 1.5 h at 0° C., the reaction mixture was poured over saturated NaHCO₃ and EtOAc. The organic phase was separated, dried over Na₂SO₄, filtered and evaporated. The title compound was purified by flash chromatography (30% to 40% EtOAc in Hexane).

### Step 10 {9-[(3,4-Dichlorophenyl)thiol-1-isopropyl-7,8-dihydro-6H-pyrido[3,4-b]pyrrolizin-8-yl}acetic acid

To a solution of ethyl {9-[(3,4-dichlorophenyl)thio]-1-isopropyl-7,8-dihydro-6H-pyrido[3,4-b]pyrrolizin-9-pyrido[3,4-b]pyrrolizin-8yl}acetate (0.23 g, 0.50 mmol) in THF (5 mL and MeOH (2.5 mL) was added 1.0 M NaOH (1.5 mL, 1.5 mmol). After stirring 18 h at RT, HOAc (0.25 mL) was added and the solvent was evaporated. The residue was taken up in EtOAc/H₂O, and the organic layer was washed with H₂O and brine. After drying (Na₂SO₄), the solution was filtered and evaporated. The residue was stirred with 1:1 EtOAc:hex to give, after filtration, the title compound as a white solid.

¹H NMR (MeOH-d₄) δ 1.14-1.26 (m, 6H), 2.47-2.56 (m, 1H), 2.56-2.64 (m, 1H), 2.94-3.05 (m, 2H), 3.81-3.89 (m, 1H), 4.22-4.30 (m, 1H), 4.33-4.44 (m, 2H), 6.93-6.99 (m, 1H), 7.14-7.19 (m, 1H), 7.33-7.39 (m, 1H), 7.54-7.59(m, 1H), 8.16-8.21(m, 1H).

The product of Step 10 was converted to its methyl ester using CH₂N₂, and the ester was subjected to HPLC separation on chiral stationary phase (chiralcel OD column 2×25 cm), elufing with 12% 2-propanol in hexane at a flow rate of 6 mL/min. Enantiomer A (less polar) has a retention time of 31.9 min and Enantiomer B (more polar) has a retention time of 35.5 min. Both A and B were hydrolyzed as in Ex. 17 Step 10 to give enantiomers A and B of the title compound.

### DP Example 22

### ((1R)-6-Fluoro-8-(methylsulfonyl)-9-{( 1S)-1-[4-(trifluoromethyl)phenyl]ethyl}-2,3,4,9-tetrahydro-1H-carbazol-1-yl)acetic acid (Compound AJ)

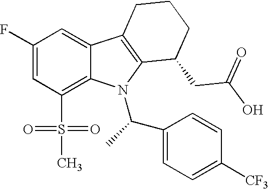

### Step 1: 2-(2-Bromo-4-fluorophenyl)hydrazinium chloride

To a suspension of 2-bromo-4-fluoroaniline in concentrated HCl (1.5M) at −10° C. was slowly added a 10.0M aqueous solution of NaNO₂ (1.1 eq). The mixture was stirred at 0° C. for 2.5 hrs. A cold (−30° C.) solution of SnCl₂ (3.8M) in concentrated HCl was then slowly added while maintaining the internal temperature below 10° C. The resulting mixture was stirred mechanically for 20 min at 10° C., then at room temperature for 1 hr. The thick slurry was filtered and the solid was air dried overnight. The solid was resuspended in cold HCl and filtered again. The dried material was suspended in Et₂O, stirred for 10 min, filtered and air dried overnight to give the title compound as a beige solid.

### Step 2: (±)-Ethyl (8-bromo-6-fluoro-2,3,4,9-tetrahydro-1H-carbazol-1-yl)acetate

To a suspension of the compound of Step 1 (1 eq) in AcOH (0.5M) was added ethyl (2-oxocyclohexyl)acetate (1 eq). The mixture was stirred at reflux for 16 hrs, cooled and AcOH was removed by evaporation under reduced pressure. The residue was diluted with EtOAc and washed with water and saturated aqueous NaHCO₃. The organic layer was dried over Na₂SO₄ and concentrated. The residue was then purified on a pad of silica gel, eluting with toluene. The filtrate was concentrated and stirred in hexanes to give, after filtration, the title compound as a white solid. MS (+APCI) m/z 354.2 (M+H)⁺.

### Step 3: (±)-Ethyl [6-fluoro-8-(methylsulfonyl)-2,3,4,9-tetrahydro-1H-carbazol-1-yl]-acetate

To a solution of the compound of Step 2 (1 eq) in anhydrous DMSO (0.28M) were added sodium methanesulphinate (3 eq) and copper iodide (3 eq). N₂ was bubbled into the mixture for 5 min and the reaction was then stirred at 100° C. under N₂ atmosphere. After 12 hrs, more sodium methanesulphinate (2 eq) and copper iodide (2 eq) were added. The mixture was stirred for a further 12 hrs at 100° C., cooled, diluted with EtOAc and 1N HCl was added to acidify the mixture. The suspension was stirred for 30 min and filtered through celite. The filtrate was washed with water, dried over Na₂SO₄ and concentrated. The residue was filtered through a pad of silica gel, eluting first with toluene to remove the non-polar impurities and then with a 2:1 mixture of hexanes/EtOAc to elute the desired product. The filtrate from the elution with the mixture of hexanes/EtOAc was concentrated to give the title compound as a pale yellow solid. MS (−APCI) m/z 352.1 (M−H)

### Step 4: Ethyl [(1R)-6-fluoro-8-(methylsulfonyl)-2,3,4,9-tetrahydro-1H-carbazol-1-yl]acetate

The racemic mixture from step 3 was resolved by preparative HPLC on a chiralpak AD preparative column eluted with a mixture of 15% iPrOH in hexane. The more polar enantiomer (longer retention time) was identified as the title compound based on the activity of the final product.

### Step 5: Ethyl [(1R)-9-[(1S)-1-(4-chlorophenyl)ethyl]-6-fluoro-8-(methylsulfonyl)-2,3,4,9-tetrahydro-1H-carbazol-1-yl]acetate

To a solution of the compound of Step 4 (1 eq), triphenylphosphine (1.5 eq) and (1R)-1-(4-chlorophenyl)ethanol (1.5 eq, prepared following the general procedure described in Reference Example 1) in THF (0.175M) was added a solution of di-tert-butyl azodicarboxylate (2.1 M in THF, 1.5 eq) over a 10 min period. The mixture was stirred at room temperature for 2 hr and concentrated. The residue was purified by silica gel flash chromatography, eluting with 7% EtOAc in toluene to give the desired product (−90% pure) which was used as such for the next reaction.

### Step 6: [(1R)-9-[( 1S)-1-(4-Chlorophenyl)ethyl]-6-fluoro-8-(methylsulfonyl)-2,3,4,9-tetrahydro-1H-carbazol-1-yl]acetic acid and [(1S)-9-[(1S)-1-(4-chlorophenyl)ethyl-6-fluoro-8-(methylsulfonyl)-2,3,4,9-tetrahydro-1H-carbazol-1-yl]acetic acid

To a solution of the compound of Step 5 in a 2:1 mixture of THF and methanol (0.1M) was added 1N aqueous LiOH (3 eq). The mixture was stirred at room temperature for 2 hr, AcOH was added and the solvent was removed by evaporation. The residue was taken up in EtOAc/H₂O and the organic layer was washed with brine, dried over Na₂SO₄, filtered and concentrated. The residue was swished in 30% EtOAc in hexane, and the product was suspended in diethyl ether and sonicated for 45 min, filtered, and dried under high vacuum at 50° C. for 24 hr to give the title compound as a white solid. MS (−APCI) m/z 462.1 (M−H)

Alternatively (±) ethyl [6-fluoro-8-(methylsulfonyl)-2,3,4,9-tetrahydro-1H-carbazol-1-yl]acetate was used for the alkylation reaction in step 5 to give a mixture of 2 diastereomers: ethyl [(1R)-9-[(1S)-1-(4-chlorophenyl)ethyl]-6-fluoro-8-(methylsulfonyl)-2,3,4,9-tetrahydro-1H-carbazol-1-yl]acetate and ethyl [(1S)-9-[(1S)-1-(4-chlorophenyl)ethyl]-6-fluoro-8-(methylsulfonyl)-2,3,4,9-tetrahydro-1H-carbazol-1-yl]acetate. The diastereomeric mixture was resolved by selective hydrolysis using the following procedure to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid.

Resolution:

The diastereomeric mixture of ethyl [(1R)-9-[(1S)-1-(4-chlorophenyl)ethyl]-6-fluoro-8-(methylsulfonyl)-2,3,4,9-tetrahydro-1H-carbazol-1-yl]acetate and ethyl [(1S)-9-[(1S)-1-(4-chlorophenyl)ethyl]-6-fluoro-8-(methylsulfonyl)-2,3,4,9-tetrahydro-1H-carbazol-1-yl]acetate (1 eq) was dissolved in a 3.5/1 mixture of THF/MeOH (0.25M) and cooled at 0° C. Aqueous LiOH 1N (1 eq) was slowly added and the mixture was stirred at 0° C. for 12 h or until almost complete hydrolysis of ethyl [(1R)-9-[(1S)-1-(4-chlorophenyl)ethyl]-6-fluoro-8-(methylsulfonyl)-2,3,4,9-tetrahydro-1H-carbazol-1-yl]acetate, the other diastereomer was only slightly hydrolyzed under these conditions. AcOH was added and the solvent was removed by evaporation. The residue was taken up in EtOAc/H₂O and the organic layer was washed with brine, dried over Na₂SO₄, filtered and concentrated. Ethyl [(1S)-9-[(1S)-1-(4-chlorophenyl)ethyl]-6-fluoro-8-(methylsulfonyl)-2,3,4,9-tetahydro-1H-carbazol-1-yl]acetate and [(1R)-9-[(1S)-1-(4-chlorophenyl)ethyl]-6-fluoro-8-(methylsulfonyl)-2,3,4,9-tetrahydro-1H-carbazol-1yl]acetic acid were separated by flash chromatography eluting with 40% EtOAc in hexanes containing 1% AcOH to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid with de>90% which was swished in 30% EtOAc in hexane to give the desired compound as a white solid with de>95%.

### Step 7: Methyl [(1R)-6-fluoro-8-(methylsulfonyl)-2,3,4,9-tetrahydro-1H-carbazol-1-yl]acetate

To a solution of [(1R)-9-[(1S)-1-(4-chlorophenyl)ethyl]-6-fluoro-8-(methylsulfonyl)-2,3,4,9-tetrahydro-1H-carbazol-1-yl]acetic acid ([α]_{D}=−226° in MeOH) in MeOH (0.1M) was added 10% palladium on carbon (10% wt/wt). A stream of N₂ was bubbled through the mixture for 5 min. The reaction was stirred at rt under H₂ atmosphere(balloon) for 24 hrs and filtered through a celite pad eluted with CH₂Cl₂. The solvents were removed by evaporation under reduced pressure and the residue was swished in MeOH to give the compound methyl [(1R)-6-fluoro-8-(methylsulfonyl)-2,3,4,9-tetrahydro-1H-carbazol-1-yl]acetate.

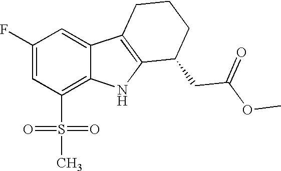

### Step 8: ((1R)-6-Fluoro-8-(methylsulfonyl)-9-{(1Ss)-1-4-(trifluoromethyl)phenyl]ethyl}-2,3,4,9-tetrahydro-1H-carbazol-1-yl)acetic acid (Compound AJ)

To a solution of the compound of step 7 (1 eq), triphenylphosphine (1.5 eq) and (1R)-1-[4-(trifluoromethyl)phenyl]ethanol (1.5 eq) in THF (0.2M) was added a solution of di-tert-butyl azodicarboxylate (1M in THF, 1.5 eq) over a 20 min period. The mixture was stirred at room temperature for 2 hr and concentrated. The residue was purified by silica gel flash chromatography eluted with 10% EtOAc in toluene to give methyl ((1R)-6-fluoro-8-(methylsulfonyl)-9-{(1S)-1-[4-(trifluoromethyl)phenyl]ethyl}-2,3,4,9-tetrahydro-1H-carbazol-1-yl)acetate (˜90% pure) which was used as such for the next reaction.

To a solution of the above ester (1 eq) in a 3.5/1 mixture of THF/MeOH (0.25M) at 0° C. was slowly added aqueous LiOH 1N (1 eq) and the mixture was stirred at 0° C. for 16 h or until almost complete hydrolysis of the ester; under these conditions, the other minor diastereomer has a much slower rate of hydrolysis. AcOH was added and the solvent was removed in vacuo. The residue was taken up in EtOAc/H₂O and the organic layer was washed with brine, dried over Na₂SO₄, filtered and concentrated. To remove the unreacted methyl ester, the residue was filtered through a pad of silica gel eluting first with 10% EtOAc/toluene and then with 60% EtOAc/toluene containing 1% of AcOH. The residue was swished in 30% EtOAc/hexane and dried under high vacuum at 50° C. for 16 hr to give the title compound as a white solid with de and ee>95% (checked by chiral BPLC). MS (−APCI) m/z 496.0 (M−H)⁻. [α]_{D}=−181° in MeOH

Biological Assays

The activity of the compounds of the present invention regarding niacin receptor affinity and function can be evaluated using the following assays:

³H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 μg/well, clean mouse at 10 ug/well, dirty preps at 30 ug/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.

2. Wash buffer and dilution buffer: Make 10 liters of ice-cold Binding Buffer:
20 mM HEPES, pH 7.4
1 MM MgCl₂
0.01% CHAPS (w/v)
use molecular grade or ddH₂O water

3. [5,6-³H]—nicotinic acid: American Radiolabeled Chemicals, Inc. (cat # ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μL total in each wells 1.5% EtOH, 50 mM tracer final.

4. Unlabeled nicotinic acid:
Make 100 mM, 10 mM, and 80 μM stocks; store at −20° C. Dilute in DMSO.

5. Preparing Plates:
1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 195 μL binding buffer to all wells of Intermediate Plates to create working solutions (250 μM →0). There will be one Intermediate Plate for each Drug Plate.

4) Transfer 5 μL from Drug Plate to the Intermediate Plate. Mix 4-5 times.

6. Procedure:
1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM ³H-nicotinic acid to all wells.
4) Seal plates, cover with aluminum foil, and shake at RT for 3-4 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of 1 mM to about 25 μM.

³⁵S-GTPγS Binding Assay:

Membranes prepared from Chinese Hamster Ovary (CHO)-Kl cells stably expressing the niacin receptor or vector control (7 μg/assay) were diluted in assay buffer (100 mM HEPES, 100 mM NaCl and 10 MM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 mM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

Membrane Preparation

Materials:

CHO-K1 cell culture medium: F-12 Kaighn's Modified Cell Culture Medium with 10% FBS, 2 mM L-Glutamine, 1 mM Sodium Pyruvate and 400 μg/ml G418

| | |
|---|---|
| Membrane Scrape Buffer: | 20 mM HEPES |
| | 10 mM EDTA, pH 7.4 |
| Membrane Wash Buffer: | 20 mM HEPES |
| | 0.1 mM EDTA, pH 7.4 |
| Protease Inhibitor Cocktail: | P-8340, (Sigma, St. Louis, MO) |

Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.
   Assay
   Materials:

Guanosine 5′-diphosphate sodium salt (GDP, Sigma-Aldrich Catalog #87127)

Guanosine 5′-[γ³⁵S] thiotriphosphate, triethylammonium salt ([³⁵S]GTPγS, Amersham Biosciences Catalog #SJ1320, ˜1000 Ci/mmol)

96 well Scintiplates (Perkin-Elmer #1450-501)

Binding Buffer: 20 mM HEPES, pH 7.4
100 mM NaCl
10 mM MgCl₂

GDP Buffer: binding buffer plus GDP, ranging from 0.4 to 40 μM, make fresh before assay

Procedure:

(total assay volume=100 μwell)

25 μL GDP buffer with or without compounds (final GDP 10 μM—so use 40 μM stock)

50 μL membrane in binding buffer (0.4 mg protein/mL)

25 μL [³⁵S]GTPγS in binding buffer. This is made by adding 5 μl [³⁵S]GTPγS stock into 10 mL binding buffer (This buffer has no GDP)
Thaw compound plates to be screened (daughter plates with 5 μL compound @ 2 mM in 100% DMSO)
Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
Thaw frozen membrane pellet on ice. (Note: they are really membranes at this point, the cells were broken in the hypotonic buffer without any salt during the membrane prep step, and most cellular proteins were washed away)
Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature. (cover plates with foil since compounds may be light sensitive)
Add 25 μL of diluted [³⁵S]GTPγS. Incubate on shaker (Lab-Line model #1314, shake at setting of 4) for 60 minutes at room temperature. Cover the plates with foil since some compounds might be light sensitive.
Assay is stopped by spinning plates sealed with plate covers at 2500 rpm for 20 minutes at 22° C.
Read on TopCount NXT scintillation counter—35S protocol.

The compounds of the invention generally have an EC₅₀ in the functional in vitro GTPγS binding assay within the range of about less than 1 uM to as high as about 100 uM.

Flushing via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott labs).

Certain compounds of the invention do not exhibit measurable in vivo vasodilation in this murine flushing model at doses up to 100 mg/kg or 300 mg/kg.

All patents, patent applications and publications that are cited herein are hereby incorporated by reference in their entirety. While certain preferred embodiments have been described herein in detail, numerous alternative embodiments are seen as falling within the scope of the invention.

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:
R¹ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
   and R′″ representing H or R″;
each R² represents H, F, Cl, Br, I or a moiety selected from the group consisting of (a), (b), (c), (d) or (e) above, or 1-2 R² groups are H, halo, C₁₋₆alkyl, OC₁₋₆alkyl, haloC₁₋₆alkyl or haloC₁₋₆alkoxy and the remaining R² groups are selected from the group consisting of (a), (b), (c), (d) or (e) above, or 1 R² group is a moiety selected from the group consisting of (a), (b), (c), (d) or (e) above, and the remaining R² groups are H or halo, or
two R² groups can be taken in combination and represent a fused phenyl ring or ring B may represent a 5-6 membered fused heterocycle containing 0-1 of S, 0-2 of O, and containing 0-4 of N, and the remaining R² group is H, halo or a moiety selected from the group consisting of (a), (b), (c), (d) or (e) above,
said phenyl ring or fused heterocycle being fused at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of:
a) OH; CO₂H; CN; NH₂; S(O)₀₋₂R^{e};
b) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN;
c) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;
d) NR′C(O)R″, NR′SO₂R″, NR′CO₂R″ and NR′C(O)NR″R′″ wherein:

What is claimed is:

1. A compound represented by formula I: 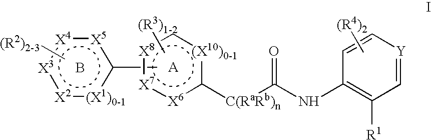
or a pharmaceutically acceptable salt or solvate thereof, wherein:
Y represents C or N;
R^{a} and R^{b} are independently H, C₁₋₃alkyl, haloC₁₋₃alkyl, OC₁₋₃alkyl, haloC₁₋₃alkoxy, OH or F;
n represents an integer of from 1 to 5;
R¹ represents —CO₂H, 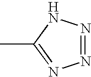
or —C(O)NHSO₂R^{c};
R^{c} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
X¹ through X¹⁰ represent C or a heteroatom selected from O, S and N, with up to 6 such heteroatoms present;
when X¹ is present, 0-2 of X¹- X⁵ represent N and 0-1 represent O or S;
when X¹ is absent, 0-3 of X²-X⁵ represent N and 0-1 represent O or S;
when X¹⁰ is present, 0-2 of X⁶-X¹⁰ represent N and 0-1 represent O or S;
when X¹⁰ is absent, 0-3 of X⁶-X⁹ represent N and 0-1 represent O or S;
when any of X¹-X¹⁰ is substituted, said X variable represents C;
when X¹⁰ is absent and at least one of X⁶-X⁹ is 0 and 2 of X⁶-X⁹ are N, and all of X¹ through X⁵ represent C, X³ is unsubstituted or is substituted with a member selected from the group consisting of: F, Br, I or a moiety selected from the group consisting of:
a) OH; CO₂H; CN; NH₂; S(O)₀₋₂R^{c};
wherein R^{c} is as previously defined;
b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, Hetcy, CN;
R¹ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄ haloalkyl, OCO₂C₁₋₄alkyl, NH₂, NHC₁₋₄alkyl, N(C₁₋₄alkyl)₂, CN, Aryl and HAR,
said Aryl and HAR being further optionally substituted with 1-3 halo, C₁₋₄alkyl, C₁₋₄alkoxy, haloC₁₋₄alkyl and haloC₁₋₄alkoxy groups;
(b) Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;
each R³ represents H, halo, C₁₋₃alkyl, OC₁₋₃alkyl, haloC₁₋₃alkyl, haloC₁₋₃alkoxy, or S(O)_{y}C₁₋₃alkyl, wherein y is 0, 1 or 2, and
each R⁴ represents H, halo, methyl, or methyl substituted with 1-3 halo groups.

2. A compound in accordance with claim 1 wherein: Y represents C.

3. A compound in accordance with claim 1 wherein R^{a} and R^{b} represent H or C₁₋₃alkyl.

4. A compound in accordance with claim 3 wherein one or both of R^{a} and R^{b} represents C₁₋₃alkyl.

5. A compound in accordance with claim 4 wherein one or both of R^{a} and R^{b} represents methyl.

6. A compound in accordance with claim 1 wherein n represents an integer 1, 2 or 3.

7. A compound in accordance with claim 6 wherein n represents 2.

8. A compound in accordance with claim 1 wherein R¹ represents CO₂H or tetrazolyl.

9. A compound in accordance with claim 8 wherein R¹ represents CO₂H.

10. A compound in accordance with claim 1 wherein R⁴ represents H or halo.

11. A compound in accordance with claim 10 wherein R⁴ represents H.

12. A compound in accordance with claim 10 wherein R⁴ represents halo.

13. A compound in accordance with claim 12 wherein R⁴ represents fluoro.

14. A compound in accordance with claim 1 wherein ring A represents a ring selected from the group consisting of: phenyl, thiazole, oxadiazole, pyrazole and thiophene.

15. A compound in accordance with claim 14 wherein ring A represents a ring selected from the group consisting of: thiazole, oxadiazole and pyrazole.

16. A compound in accordance with claim 1 wherein ring B represents a ring selected from the group consisting of: phenyl, pyridyl, pyrimidinyl, oxadiazolyl, faranyl, pyrazolyl and oxazolyl.

17. A compound in accordance with claim 1 wherein ring B represents a ring selected from the group consisting of: phenyl, pyridine, pyrimidine, oxadiazole, furan and pyrazole.

18. A compound in accordance with claim 1 wherein ring B represents a phenyl, pyridyl, pyrimidinyl, oxazolyl or furanyl ring.

19. A compound in accordance with claim 16 wherein ring B represents a phenyl or pyridyl ring.

20. A compound in accordance with claim 19 wherein ring B represents a pyridyl ring.

21. A compound in accordance with claim 1 wherein each R² represents H, F, Cl, or a moiety selected from the group consisting of
a) OH; CO₂H; CN; NH₂;
b) C₁₋₃ alkyl and OC₁₋₃alkyl, said group being optionally substituted with 1-3 groups, 1-3 of which are halo and 1 of which is selected from: OH, CO₂H, CO₂C₁₋₄alkyl, CO₂C₁₋₄ haloalkyl, NH₂, NHCH₃ and N(CH₃)₂;
c) NHCH₃ and N(CH₃)₂;
d) C(O)NH₂, C(O)NHCH₃, C(O)N(CH₃)₂, C(O)NHOCH₃ and C(O)N(CH₃)(OCH₃);
e) NR′C(O)R″, NR′SO₂R″, NR′CO₂R″ and NR′C(O)NR″R′″ wherein:
R′ represents H, CH₃ or haloC₁₋₂alkyl,
R″ represents (a) C₁₋₂alkyl optionally substituted with 1-3 groups, 0-3 of which are halo, and 0-1 of which are selected from the group consisting of: OCH₃, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl,
said Aryl being further optionally substituted with 1-3 halo, CH₃, OCH₃, haloC₁₋₂alkyl and haloC₁₋₂alkoxy groups;
(b) Aryl optionally substituted with 1-3 halo, CH₃, OCH₃, C₁₋₂alkoxy, haloC₁₋₂alkyl and haloC₁₋₂alkoxy groups;
and R′″ represents H or R″.

22. A compound in accordance with claim 1 wherein two R² taken in combination represent a fused phenyl ring or a 5-6 membered fused heterocycle containing 0-1 of S, 0-2 of O, and containing 0-4 of N, and the remaining R² group is H, F, Cl, or a moiety selected from the group consisting of
a) OH; CO₂H; CN; NH₂;
b) C₁₋₃ alkyl and OC₁₋₃alkyl, said group being optionally substituted with 1-3 groups, 1-3 of which are halo and 1 of which is selected from: OH, CO₂H, CO₂C₁₋₄alkyl, CO₂C₁₋₄ haloalkyl, NH₂, NHCH₃ and N(CH₃)₂;
c) NHCH₃ and N(CH₃)₂;
d) C(O)NH₂, C(O)NHCH₃, C(O)N(CH₃)₂, C(O)NHOCH₃ and C(O)N(CH₃)(OCH₃);
e) NR′C(O)R″, NR′SO₂R″, NR′CO₂R″ and NR′C(O)NR″R′″ wherein:
R′ represents H, CH₃ or haloC₁₋₂alkyl,
R″ represents (a) C₁₋₂alkyl optionally substituted with 1-3 groups, 0-3 of which are halo, and 0-1 of which are selected from the group consisting of: OCH₃, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl,
said Aryl being further optionally substituted with 1-3 halo, CH₃, OCH₃, haloC₁₋₂alkyl and haloC₁₋₂alkoxy groups;
(b) Aryl optionally substituted with 1-3 halo, CH₃, OCH₃, C₁₋₂alkoxy, haloC₁₋₂alkyl and haloC₁₋₂alkoxy groups;
and R′″ represents H or R″;
said fused phenyl ring or heterocycle being fused at any available point and being optionally substituted with 1-3 halo, C₁₋₂alkyl or haloC₁₋₂alkyl groups, or 1-2 OC₁₋₂alkyl or haloOC₁₋₂alkyl groups, or 1 moiety selected from the group consisting of:
a) OH; CO₂H; CN; NH₂;
b) NHCH₃ and N(CH₃)₂, the alkyl portions of which are optionally substituted with 1-3 groups, 1-3 of which are halo and 1 of which is selected from: OH, CO₂H, CO₂C₁₋₂alkyl, CO₂C₁₋₂haloalkyl, OCO₂C₁₋₂alkyl, NH₂, NHCH₃, N(CH₃)₂, CN;
c) C(O)NH₂, C(O)NHCH₃, C(O)N(CH₃)₂, C(O)NHOCH₃ and C(O)N(CH₃)(OCH₃), the alkyl portions of which are optionally substituted as set forth in (b) above;
d) NR′C(O)R″, NR′SO₂R″, NR′CO₂R″ and NR′C(O)NR″R′″ wherein:
R′ represents H, C₁₋₂alkyl or haloC₁₋₂alkyl,
R″ represents (a) C₁₋₈alkyl optionally substituted with 1-4 groups, 0-4 of which are halo, and 0-1 of which are selected from the group consisting of: OC₁₋₃alkyl, OH, CO₂H, CO₂C₁₋₂alkyl, CO₂C₁₋₂ haloalkyl, OCO₂C₁₋₂alkyl, NH₂, NHCH₃, N(CH₃)₂, CN and Aryl HAR,
said Aryl being further optionally substituted with 1-3 halo, CH₃, OCH₃, haloC₁₋₂alkyl and haloC₁₋₂alkoxy groups;
(b) Aryl or HAR, said Aryl and HAR being further optionally substituted with 1-3 halo, CH₃, OCH₃, haloC₁₋₂alkyl and haloC₁₋₂alkoxy groups;
and R′″ representing H or R″.

23. A compound in accordance with claim 1 selected from Table 1 below:
TABLE 1 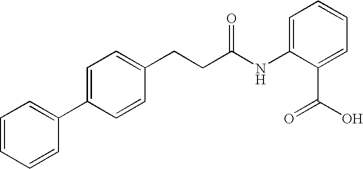 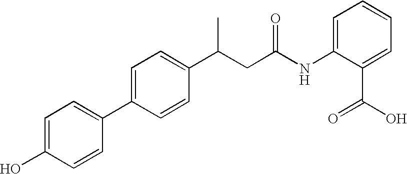 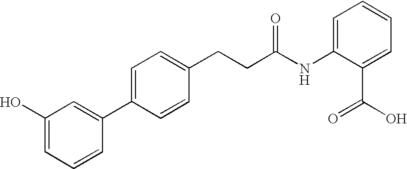 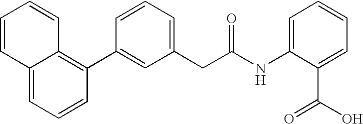 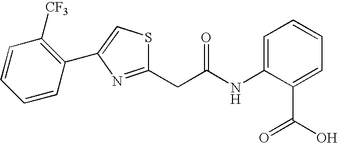 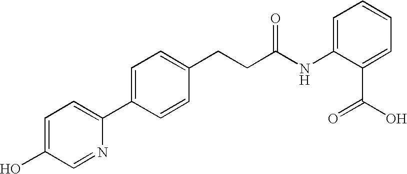 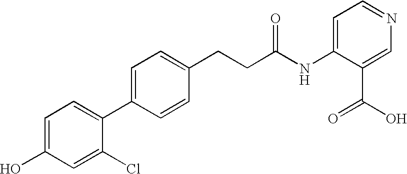 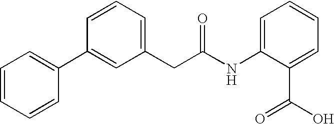 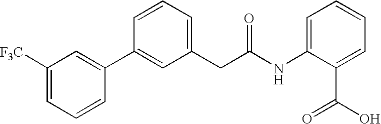 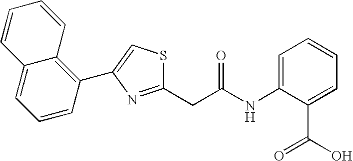 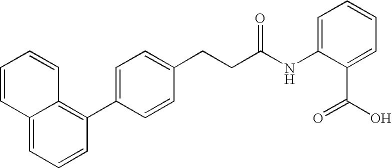 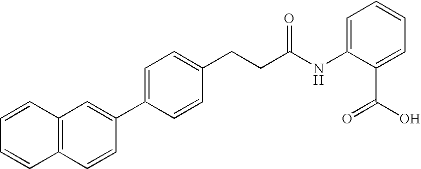 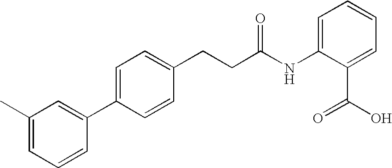 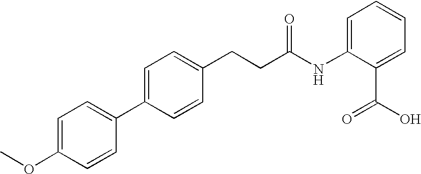 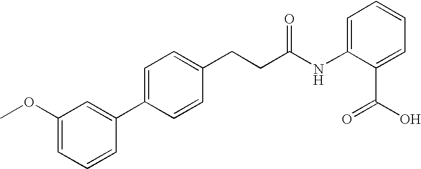 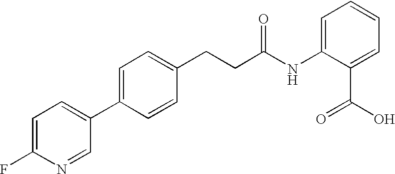 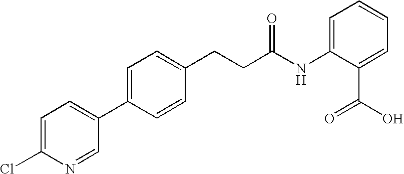 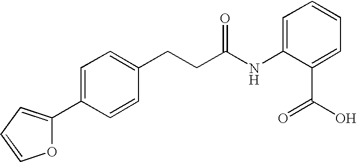 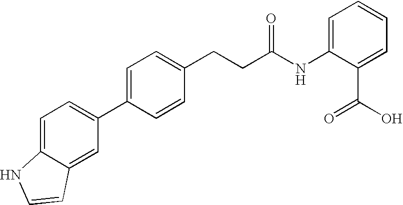 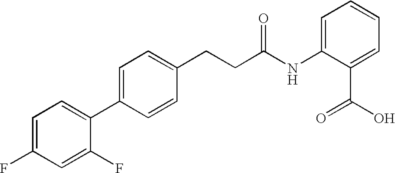 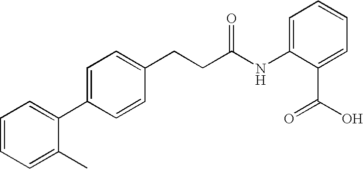 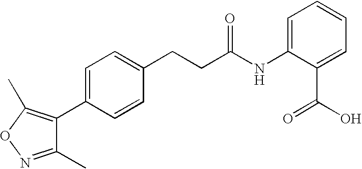 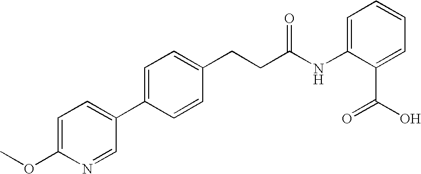 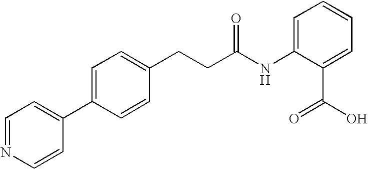 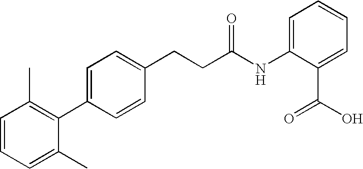 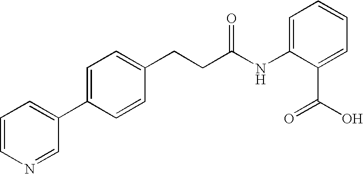 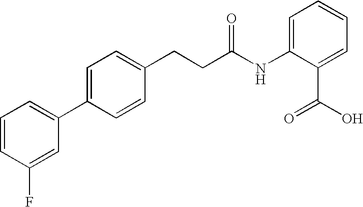 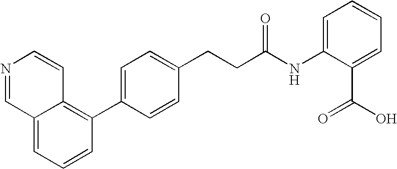 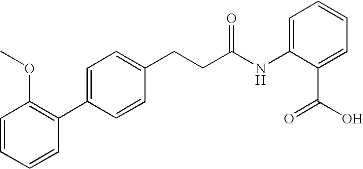 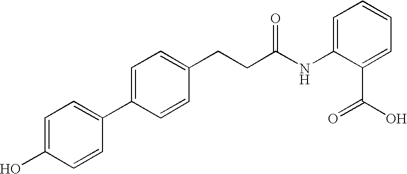 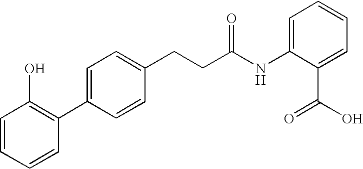 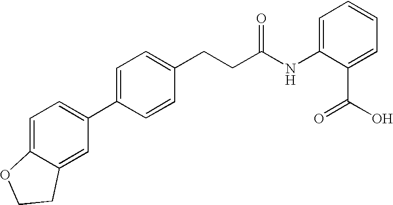 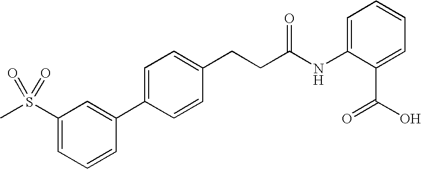 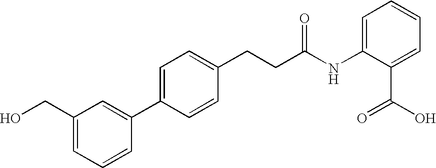 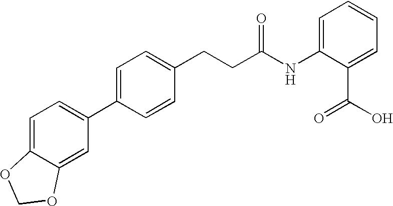 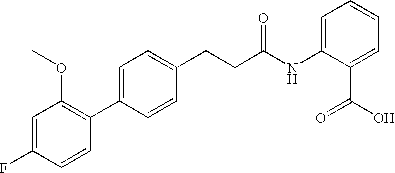 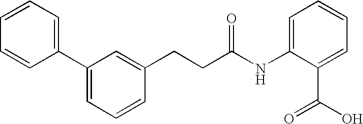 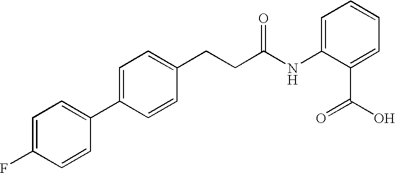 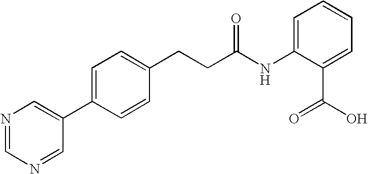 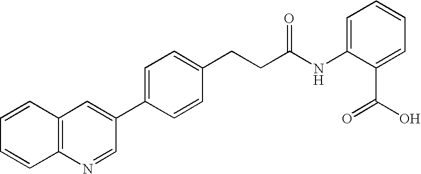 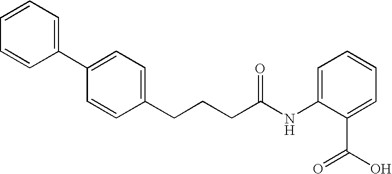 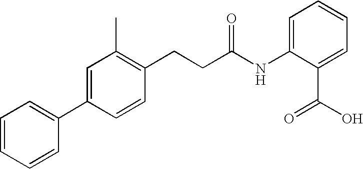 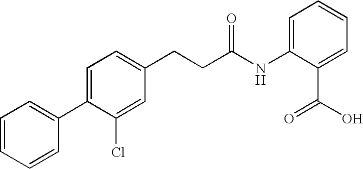 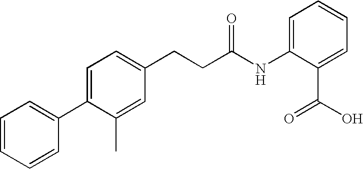 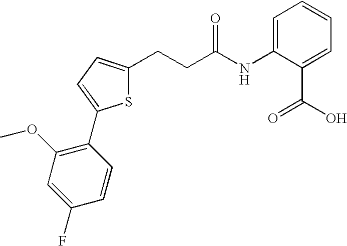 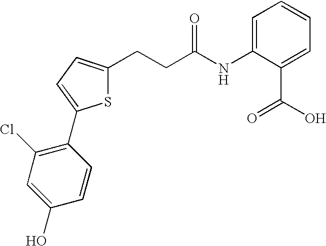 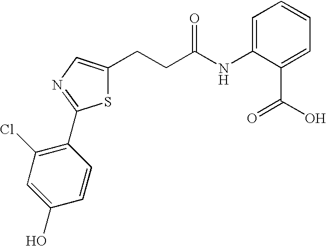 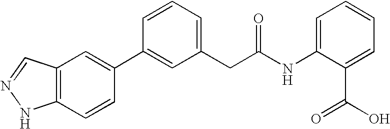 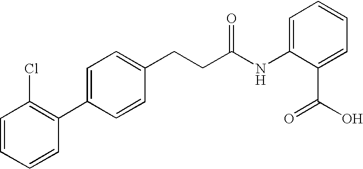 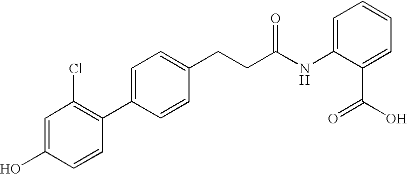 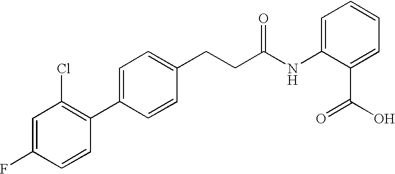 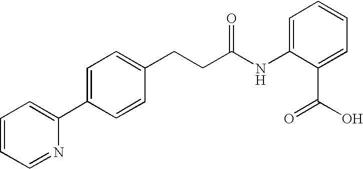 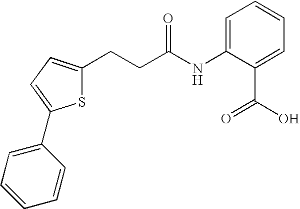 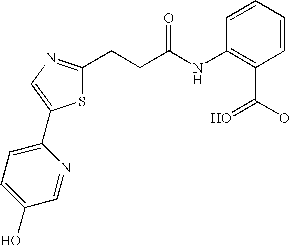 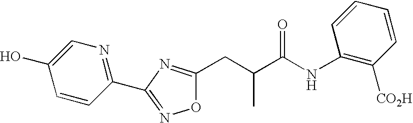 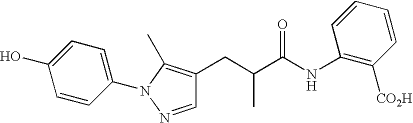 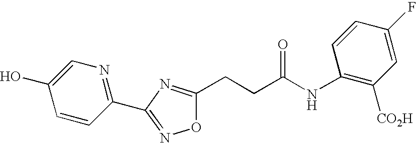 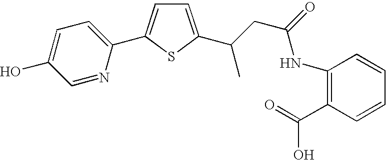 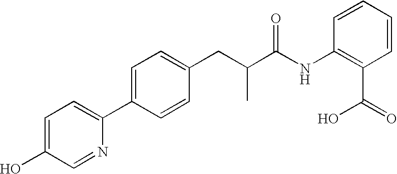 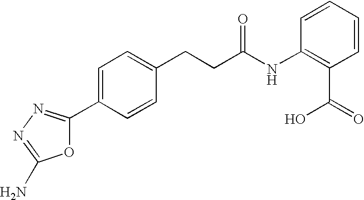 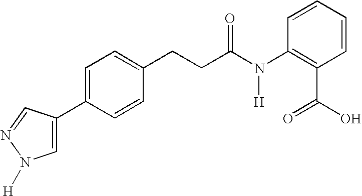 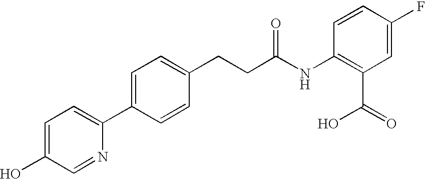 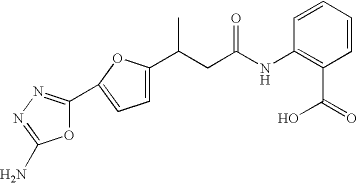
or a pharmaceutically acceptable salt or solvate thereof.

24. A pharmaceutical composition comprising a compound in accordance with claim 1 in combination with a pharmaceutically acceptable carrier.

25. A method of treating atherosclerosis in a human patient in need of such treatment comprising administering to the patient a compound of claim 1 in an amount that is effective for treating atherosclerosis.

26. A method of treating dyslipidemia in a human patient in need of such treatment comprising administering to the patient a compound of claim 1 in an amount that is effective for treating dyslipidemias.
